(19) United States
(12) Patent Application Publication
(10) Pub. No.: US 2025/0043002 A1
(43) Pub. Date: Feb. 6, 2025

(54) BISPECIFIC PD-L1xCD28 ANTIBODIES AND METHODS OF USE THEREOF

(71) Applicant: Regeneron Pharmaceuticals, Inc., Tarrytown, NY (US)

(72) Inventors: Saida Dadi-Mehmetaj, Ardsley, NY (US); Lauric Haber, Rye Brook, NY (US); Jessica Kirshner, New York, NY (US); Lauren Canova-Kleinman, White Plains, NY (US); Erica Ullman, Yorktown Heights, NY (US)

(21) Appl. No.: 18/766,785

(22) Filed: Jul. 9, 2024

Related U.S. Application Data
(60) Provisional application No. 63/645,552, filed on May 10, 2024.
(60) Provisional application No. 63/512,718, filed on Jul. 10, 2023.

(51) Int. Cl. C07K 16/28; A61K 39/00; A61K 45/06; A61P 35/00; C12N 15/85

(52) U.S. Cl. CPC C07K 16/2818; A61K 45/06; A61P 35/00; C07K 16/2827; C12N 15/85; A61K 2039/507; C07K 2317/14; C07K 2317/21; C07K 2317/31; C07K 2317/53; C07K 2317/565; C07K 2317/71; C07K 2317/92

(57) ABSTRACT

The present disclosure provides bispecific antigen-binding molecules that bind to PD-L1 and CD28 (PD-L1×CD28). In certain embodiments, the present disclosure provides for a bispecific PD-L1×CD28 antigen-binding molecule (or antibody) or antigen-binding fragment thereof comprising a first antigen-binding domain that specifically binds CD28, and a second antigen-binding domain that specifically binds PD-L1. In certain embodiments, the bispecific antigen-binding molecules of the present disclosure bind CD28 on T-cells with the first antigen-binding domain and PD-L1 expressed on tumor cells or antigen presenting cells with the second antigen-binding domain. In certain embodiments, the bispecific antigen-binding molecules are capable of inhibiting growth of a tumor. The bispecific antigen-binding molecules of this disclosure are useful for treatment of cancer.

### FIELD

The present disclosure relates to antibodies that bind to PD-L1 and CD28 and methods of use thereof, e.g., for treating or preventing cancer.

### SEQUENCE LISTING

The instant application contains a Sequence Listing which has been submitted electronically in XML format and is hereby incorporated by reference in its entirety. Said XML copy, created on May 10, 2024, is named SeqList11374.XML and is 103,464 bytes in size. The sequence listing is part of the specification and is incorporated in its entirety by reference herein. The following sequences are below the minimum length permitted under ST.26 format for sequence listings: gctgcatcc (SEQ ID NO: 21), AAS (SEQ ID NO: 22), ggggcaagt (SEQ ID NO: 69), and GAS (SEQ ID NO: 70).

### BACKGROUND

The ability of T-cells to recognize and kill their cellular targets—such as virally-infected cells or tumor cells—depends on a coordinated set of interactions. Foremost among these is the recognition and binding of the target cell by the T-cell Receptor (TCR) complex (which includes the associated CD3 γ, δ, ε and ζ chains), and this interaction has been referred to as “signal 1” for T-cell activation. The TCR recognizes a viral or tumor peptide presented on the groove of an MHC protein expressed on the surface of the target cell. Because such binding is generally of low-affinity, successful triggering of “signal 1” requires clustering of many TCR complexes along the interface between the T-cell and its target cell; this interface has been referred to as the “immune synapse”. T-cell activation can be further promoted by additional interactions. For example, T-cells have a molecule referred to as CD28 on their surface, which can provide a co-stimulatory “signal 2” to augment the activation via the TCR complex. When a T-cell recognizes its target cell via its TCR complex, and then also engages “signal 2” via CD28 binding to its cognate ligand(s) on the target cell, T-cell activation is enhanced; as with “signal 1”, CD28-mediated “signal 2” is thought to occur via co-clustering at the immune synapse.

Agonistic anti-CD28 (monoclonal antibodies) (mAbs) can be applied in sustained ex vivo expansion of cultured T-cells; however, the use of antibodies against CD28 has been discouraged as a result of a series of acute and serious adverse events in a phase I clinical trial where super agonist anti-CD28 mAb was tested systemically (Hunig, Nat. Rev. Immunol. 2012; 12:317-318). Localized or targeted use of anti-CD28 mAb can be used for promotion of antitumor immunity with less risk. Jung et al., Int J Cancer. 2001-1-15; 91(2):225-30.

Programmed death ligand 1 (PD-L1), a ligand of programmed death (PD-1 or PD1), is expressed on antigen-presenting cells such as activated monocytes and dendritic cells, as well as on certain cancer cells. It is known that that stimulation by PD-L1 suppresses activation (cellular proliferation and induction of various cytokine production) of PD-1 expressing T lymphocytes.

Monoclonal antibodies (mAbs) aimed at enhancing T-cell activation are under clinical development as anti-tumor therapeutics. The majority of current treatments, however, have a difficult time overcoming the inhibitory nature of the tumor microenvironment, thus failing to generate efficient tumor-specific T-cell activation and subsequent tumor cell killing. Several blocking mAbs directed against checkpoint inhibitors such as CTLA-4 (cytotoxic T lymphocyte-associated protein) and programmed cell death 1 (PD-1)/programmed cell death ligand 1 (PD-L1) have been clinically approved for melanoma, renal cell carcinoma, non-small lung cancer and advanced metastatic cutaneous squamous cell carcinoma. Blocking PD-1 releases the break on T-cell activation, but its efficacy as a single agent often it is not sufficient to achieve tumor clearance and durable anti-tumor responses. Therefore, there is a need for additional treatments for cancer, such as improved immunotherapeutic agents.

### SUMMARY

The present disclosure provides bispecific antigen-binding molecules, including bispecific antibodies, that bind Cluster of Differentiation 28 (CD28) and programmed death ligand 1 (PD-L1) (“PD-L1×CD28” or “CD28×PD-L1”). The bispecific antigen-binding molecules of the present disclosure provide a pan-tumor costimulatory approach (tumor-agnostic targeting) whereby inhibitory checkpoint PD-L1 which is present across various tumor types is converted to a costimulatory signal activating CD28 on T cells. The PD-L1×CD28 bispecific antigen-binding molecules block PD-L1 interaction with PD-1 as well as PD-L1 interaction with CD80, thus protecting CD80 from CTLA4 trogocytosis and making it available for binding to CD28 which provides co-stimulatory signal to T cells. The PD-L1×CD28 bispecific antigen-binding molecules of the present disclosure bind to and engage PD-L1 expressing tumor cells, APCs and other tumor-infiltrating immune cells, thus providing a broad tumor-agnostic targeting as compared to a bispecific targeting CD28 and a tumor associated antigen (TAA). In certain embodiments, the bispecific antigen-binding molecules enhance the T cell mediated killing of tumor cells expressing PD-L1 and a TAA, when used in combination with a CD3-based bispecific targeted to that TAA. The bispecific antigen-binding molecules bind to CD28 with low affinity which in turn results in decreased cytokine release. Thus, combining this class of CD28-based bi-specifics with a PD-1 inhibitory antibody and/or a bispecific TAA×CD3 antibody may provide safe and efficacious anti-tumor therapy against many tumor types.

In one aspect, the present disclosure provides an isolated bispecific antigen-binding molecule comprising a first antigen-binding domain that specifically binds human CD28 with a KD of less than about 3×10⁻⁸ M as measured by surface plasmon resonance at 25° C.; and a second antigen-binding domain that specifically binds a programmed death-ligand 1 (PD-1) with a KD of less than about 2×10⁻¹⁰ M as measured by surface plasmon resonance at 25° C.

In some embodiments, the bispecific antigen-binding molecule binds to the surface of human T cells with an EC₅₀ of less than about 2×10⁻⁸ M as measured by an in vitro FACS binding assay. In some embodiments, the bispecific antigen-binding molecule binds to the surface of human T cells with an EC₅₀ of less than about 8×10⁻⁸ M as measured by an in vitro FACS binding assay.

In some embodiments, the bispecific antigen-binding molecule binds to the surface of a cell expressing PD-L1 with an EC₅₀ of less than about 3×10⁻⁹ M as measured by an in vitro FACS binding assay.

In some embodiments, the bispecific antigen-binding molecule blocks PD-L1 binding to PD-1 with an IC₅₀ of less than about 1.3 nM as measured by an ELISA-based blocking assay.

In certain embodiments, the isolated bispecific antigen-binding molecule demonstrates a costimulatory effect when used in conjunction with an anti-Mucine 16 (MUC16)×CD3 bispecific antibody and tested on tumor cells expressing PD-L1 and MUC16. In one embodiment, the costimulatory effect is shown by one or more of the following: (a) the ability to activate and direct human T cells to kill a target cell expressing PD-L1; (b) the ability to upregulate PD-1 on T cells; (c) the ability to increase the release of the cytokines IFN gamma and TNF from PBMC; (d) the ability to deplete tumor cells; or (f) the ability to enhance tumor clearance. In another embodiment, the costimulatory effect is further shown by one or more of the following: (g) activation of NFκB activity in a T cell/APC luciferase-based reporter assay; or (h) measurement of IL-2 cytokine production using a primary CD4⁺ T cell/APC functional assay.

In some embodiments, the bispecific antigen-binding molecule, in combination with a bispecific MUC16×CD3 antibody mediates in vitro T cell killing of OVCAR-3 cells expressing PD-L1 with an EC₅₀ of less than about 10⁻¹⁰ M.

For example, in an embodiment, the bi-specific antigen-binding molecule (e.g., antibody or antigen-binding fragment thereof) that binds PD-L1 and CD28 includes: (1) a PD-L1 binding arm comprising: (a) a heavy chain variable region thereof comprising an amino acid sequence having at least 90%, 91%, 92%, 93%, 94%, 95%, 96%, 97%, 98%, or 99% amino acid sequence identity to, comprising, or consisting of the amino acid sequence set forth in SEQ ID NOs: 2 and 42, or a variant thereof; and/or (b) a light chain variable region thereof comprising an amino acid sequence having at least 90%, 91%, 92%, 93%, 94%, 95%, 96%, 97%, 98%, or 99% amino acid sequence identity to, comprising, or consisting of the amino acid sequence set forth in SEQ ID NOs: 18 and 58, or a variant thereof; and/or (2) a CD28 binding arm comprising: (c) a heavy chain immunoglobulin or variable region thereof comprising an amino acid sequence having at least 90%, 91%, 92%, 93%, 94%, 95%, 96%, 97%, 98%, or 99% amino acid sequence identity to, comprising, or consisting of the amino acid sequence set forth in SEQ ID NOs: 10, 32 and 50, or a variant thereof; and/or (d) a light chain immunoglobulin or variable region thereof comprising an amino acid sequence having at least 90%, 91%, 92%, 93%, 94%, 95%, 96%, 97%, 98%, or 99% amino acid sequence identity to, comprising, or consisting of the amino acid sequence set forth in SEQ ID NOs: 18 and 58.

In an embodiment, the bispecific antigen-binding protein (e.g., antibody or antigen-binding fragment thereof) that binds PD-L1 and CD28 includes:
(1) a PD-L1 binding arm comprising: (a) a heavy chain immunoglobulin or variable region thereof comprising the HCDR1, HCDR2 and HCDR3 of a heavy chain variable region comprising an amino acid sequence set forth in SEQ ID NO: 2 or 42, and at least 90%, 91%, 92%, 93%, 94%, 95%, 96%, 97%, 98%, or 99% amino acid sequence identity to, comprising, or consisting of the amino acid sequence set forth in SEQ ID NO: 2 or 42, respectively; and/or (b) a light chain immunoglobulin or variable region thereof comprising the LCDR1, LCDR2 and LCDR3 of a light chain variable region comprising an amino acid sequence set forth in SEQ ID NO: 18 or 58, and at least 90%, 91%, 92%, 93%, 94%, 95%, 96%, 97%, 98%, or 99% amino acid sequence identity to, comprising, or consisting of the amino acid sequence set forth in SEQ ID NO: 18 or 58, respectively; and a CD28 binding arm; or (2) a CD28 binding arm comprising: (c) a heavy chain immunoglobulin or variable region thereof comprising the HCDR1, HCDR2 and HCDR3 of a heavy chain variable region comprising an amino acid sequence set forth in SEQ ID NO: 10, 32 or 50, and at least 90%, 91%, 92%, 93%, 94%, 95%, 96%, 97%, 98%, or 99% amino acid sequence identity to, comprising, or consisting of the amino acid sequence set forth in SEQ ID NO: 10, 32 or 50, respectively; and/or (d) a light chain immunoglobulin or variable region thereof comprising the LCDR1, LCDR2 and LCDR3 of a light chain variable region comprising an amino acid sequence set forth in SEQ ID NO: 18 or 58, and at least 90%, 91%, 92%, 93%, 94%, 95%, 96%, 97%, 98%, or 99% amino acid sequence identity to, comprising, or consisting of the amino acid sequence set forth in SEQ ID NO: 18 or 58, respectively; and a PD-L1 binding arm.

In some embodiments, the first antigen-binding domain comprises: (a) three heavy chain complementarity determining regions (HCDR1, HCDR2 and HCDR3) contained within a heavy chain variable region (HCVR) comprising the amino acid sequence selected from the group consisting of SEQ ID NOs: 50, 32 and 10, or a variant thereof; and (b) three light chain complementarity determining regions (LCDR1, LCDR2 and LCDR3) contained within a light chain variable region (LCVR) comprising the amino acid sequence selected from the group consisting of SEQ ID NOs: 58 and 18, or a variant thereof.

In some embodiments, the isolated bispecific antigen-binding molecule comprises a HCDR1 comprising the amino acid sequence selected from the group consisting of SEQ ID NOs: 52, 34 and 12, a HCDR2 comprising the amino acid sequence selected from the group consisting of SEQ ID NOs: 54, 36 and 14, and a HCDR3 comprising the amino acid sequence selected from the group consisting of SEQ ID NOs: 56, 38 and 16.

In some embodiments, the isolated bispecific antigen-binding molecule comprises a LCDR1 comprising the amino acid sequence selected from the group consisting of SEQ ID NOs: 60 and 20, a LCDR2 comprising the amino acid sequence selected from the group consisting of SEQ ID NOs: 62 and 22, and a LCDR3 comprising the amino acid sequence selected from the group consisting of SEQ ID NO: 64 and 24.

In some embodiments, the first antigen-binding domain comprises a HCVR comprising the amino acid sequence of SEQ ID NO: 50, or a variant thereof, and a LCVR comprising the amino acid sequence of SEQ ID NO: 58, or a variant thereof.

In some embodiments, the first antigen-binding domain comprises a HCVR comprising the amino acid sequence of SEQ ID NO: 32, or a variant thereof, and a LCVR comprising the amino acid sequence of SEQ ID NO: 18, or a variant thereof.

In some embodiments, the first antigen-binding domain comprises a HCVR comprising the amino acid sequence of SEQ ID NO: 10, or a variant thereof, and a LCVR comprising the amino acid sequence of SEQ ID NO: 18, or a variant thereof.

In some embodiments, the second antigen-binding domain comprises: (a) three heavy chain complementarity determining regions (HCDR1, HCDR2 and HCDR3) contained within a heavy chain variable region (HCVR) comprising the amino acid sequence selected from the group consisting of SEQ ID NOs: 42 and 2, or a variant thereof; and (b) three light chain complementarity determining regions (LCDR1, LCDR2 and LCDR3) contained within a light chain variable region (LCVR) comprising the amino acid sequence selected from the group consisting of SEQ ID NOs: 58 and 18, or a variant thereof.

In some embodiments, the second antigen-binding domain comprises: (a) a HCDR1 comprising the amino acid sequence of SEQ ID NO: 44 or SEQ ID NO: 4; (b) a HCDR2 comprising the amino acid sequence of SEQ ID NO: 46 or SEQ ID NO: 6; and (c) a HCDR3 comprising the amino acid sequence of SEQ ID NO: 48 or SEQ ID NO: 8.

In some embodiments, the second antigen-binding domain comprises a LCDR1 comprising the amino acid sequence selected from the group consisting of SEQ ID NOs: 60 and 20, a LCDR2 comprising the amino acid sequence selected from the group consisting of SEQ ID NOs: 62 and 22, and a LCDR3 comprising the amino acid sequence selected from the group consisting of SEQ ID NO: 64 and 24.

In some embodiments, the second antigen-binding domain comprises: (a) HCDR1, HCDR2, HCDR3 domains, respectively, comprising the amino acid sequences of SEQ ID NOs: 44, 46, 48; and LCDR1, LCDR2, LCDR3 domains, respectively, comprising the amino acid sequences of SEQ ID NOs: 60, 62, 64; or (b) HCDR1, HCDR2, HCDR3 domains, respectively, comprising the amino acid sequences of SEQ ID NOs: 4, 6, 8; and LCDR1, LCDR2, LCDR3 domains, respectively, comprising the amino acid sequences of SEQ ID NOs: 20, 22, 24.

In some embodiments, the second antigen-binding domain comprises: (a) a HCVR comprising the amino acid sequence of SEQ ID NO: 42, or a variant thereof, and a LCVR comprising the amino acid sequence of SEQ ID NO: 58, or a variant thereof; or (b) a HCVR comprising the amino acid sequence of SEQ ID NO: 2, or a variant thereof, and a LCVR comprising the amino acid sequence of SEQ ID NO: 18, or a variant thereof.

In some aspects, the present disclosure provides an isolated bispecific antigen-binding molecule, comprising: (a) a first antigen-binding domain that specifically binds human CD28 wherein the first antigen-binding domain comprises HCDR1, HCDR2, HCDR3 domains, respectively, comprising the amino acid sequences of SEQ ID NOs: 52,54, 56, and LCDR1, LCDR2, LCDR3 domains, respectively, comprising the amino acid sequences of SEQ ID NOs: 60,62,64; and (b) a second antigen-binding domain that specifically binds human PD-L1 wherein the second antigen-binding domain comprises HCDR1, HCDR2, HCDR3 domains, respectively, comprising the amino acid sequences of SEQ ID NOs: 44,46,48, and LCDR1, LCDR2, LCDR3 domains, respectively, comprising the amino acid sequences of SEQ ID NOs: 60, 62, 64.

In some aspects, the present disclosure provides an isolated bispecific antigen-binding molecule, comprising: (a) a first antigen-binding domain that specifically binds human CD28 wherein the first antigen-binding domain comprises HCDR1, HCDR2, HCDR3 domains, respectively, comprising the amino acid sequences of SEQ ID NOs: 34, 36, 38, and LCDR1, LCDR2, LCDR3 domains, respectively, comprising the amino acid sequences of SEQ ID NOs: 20, 22, 24; and (b) a second antigen-binding domain that specifically binds human PD-L1 wherein the second antigen-binding domain comprises HCDR1, HCDR2, HCDR3 domains, respectively, comprising the amino acid sequences of SEQ ID NOs: 4, 6, 8, and LCDR1, LCDR2, LCDR3 domains, respectively, comprising the amino acid sequences of SEQ ID NOs: 20, 22, 24.

In some aspects, the present disclosure provides an isolated bispecific antigen-binding molecule, comprising: (a) a first antigen-binding domain that specifically binds human CD28 wherein the first antigen-binding domain comprises HCDR1, HCDR2, HCDR3 domains, respectively, comprising the amino acid sequences of SEQ ID NOs: 12,14,16, and LCDR1, LCDR2, LCDR3 domains, respectively, comprising the amino acid sequences of SEQ ID NOs: 20, 22, 24; and (b) a second antigen-binding domain that specifically binds human PD-L1 wherein the second antigen-binding domain comprises HCDR1, HCDR2, HCDR3 domains, respectively, comprising the amino acid sequences of SEQ ID NOs: 4,6,8, and LCDR1, LCDR2, LCDR3 domains, respectively, comprising the amino acid sequences of SEQ ID NOs: 20, 22, 24.

In some embodiments, the isolated bispecific antigen-binding molecule comprises: (a) a first antigen-binding domain that comprises a HCVR comprising the amino acid sequence of SEQ ID NO: 50, and a LCVR comprising the amino acid sequence of SEQ ID NO: 58; and (b) a second antigen-binding domain that comprises a HCVR comprising the amino acid sequence of SEQ ID NO: 42, and a LCVR comprising the amino acid sequence of SEQ ID NO: 58.

In some embodiments, the isolated bispecific antigen-binding molecule comprises: (a) a first antigen-binding domain that comprises a HCVR comprising the amino acid sequence of SEQ ID NO: 32, and a LCVR comprising the amino acid sequence of SEQ ID NO: 18; and (b) a second antigen-binding domain that comprises a HCVR comprising the amino acid sequence of SEQ ID NO: 2, and a LCVR comprising the amino acid sequence of SEQ ID NO: 18.

In some embodiments, the isolated bispecific antigen-binding molecule comprises: (a) a first antigen-binding domain that comprises a HCVR comprising the amino acid sequence of SEQ ID NO: 10, and a LCVR comprising the amino acid sequence of SEQ ID NO: 18; and (b) a second antigen-binding domain that comprises a HCVR comprising the amino acid sequence of SEQ ID NO: 2, and a LCVR comprising the amino acid sequence of SEQ ID NO: 18.

In certain aspects, the present disclosure provides an isolated bispecific antigen-binding molecule that competes for binding to PD-L1 or binds to the same epitope on PD-L1 as a reference antibody, wherein the reference antibody comprises a first antigen-binding domain comprising an HCVR/LCVR pair comprising the amino acid sequences selected from the group consisting of SEQ ID NOs: 50/58, 32/18 and 10/18 and a second antigen-binding domain comprising an HCVR/LCVR pair comprising the amino acid sequences selected from the group consisting of SEQ ID NOs: 42/58 and 2/18.

In certain aspects, the present disclosure provides an isolated bispecific antigen-binding molecule that competes for binding to human CD28 or binds to the same epitope on human CD28 as a reference antibody, wherein the reference antibody comprises a first antigen-binding domain comprising an HCVR/LCVR pair comprising the amino acid sequences selected from the group consisting of SEQ ID NOs: 50/58, 32/18 and 10/18 and a second antigen-binding domain comprising an HCVR/LCVR pair comprising the amino acid sequences selected from the group consisting of SEQ ID NOs: 42/58 and 2/18.

In some embodiments, the isolated bispecific antigen-binding molecule is a human bispecific antigen-binding molecule.

In some embodiments, the isolated bispecific antigen-binding molecule is a bispecific antibody.

In some embodiments, the antibody comprises a human IgG heavy chain constant region attached, respectively, to the HCVR of each of the first antigen-binding domain and the second antigen-binding domain. In some embodiments, the heavy chain constant region is of isotype IgG1. In some embodiments, the heavy chain constant region is of isotype IgG4.

In some embodiments, the heavy chain constant region attached to the HCVR of the first antigen-binding domain, or the heavy chain constant region attached to the HCVR of the second antigen-binding domain, but not both, contains an amino acid modification that reduces Protein A binding relative to a heavy chain of the same isotype without the modification.

In some embodiments, the modification comprises a H435R substitution (EU numbering) in a heavy chain of isotype IgG1 or IgG4.

In some embodiments, the modification comprises a H435R substitution and a Y436F substitution (EU numbering) in a heavy chain of isotype IgG1 or IgG4.

In some embodiments, the bispecific antibody comprises a chimeric hinge that reduces Fcγ receptor binding relative to a wild-type hinge of the same isotype.

In some embodiments, the antibody comprises a first heavy chain containing the HCVR of the first antigen-binding domain and a second heavy chain containing the HCVR of the second antigen-binding domain, wherein the first heavy chain comprises the amino acid sequence selected from the group consisting of SEQ ID NOs: 68, 40 and 28; and the second heavy chain comprises the amino acid sequence selected from the group consisting of SEQ ID NOs: 66 and 26.

In some embodiments, the antibody comprises a common light chain containing the LCVR of the first and second antigen-binding domains, wherein the common light chain comprises the amino acid sequence selected from the group consisting of SEQ ID NOs: 70 and 30.

In some embodiments, the antibody comprises a first heavy chain containing the HCVR of the first antigen-binding domain and a second heavy chain containing the HCVR of the second antigen-binding domain, wherein the first heavy chain comprises the amino acid sequence of SEQ ID NO: 68 and the second heavy chain comprises the amino acid sequence of SEQ ID NO: 66.

In some embodiments, the antibody comprises a common light chain containing the LCVR of the first and second antigen-binding domains, wherein the common light chain comprises the amino acid sequence of SEQ ID NOs: 70.

In some aspects the disclosure provides a bispecific antibody comprising a first antigen-binding domain that binds specifically to human CD28 and a second antigen-binding domain that binds specifically to human PD-L1, wherein the bispecific antibody comprises a first heavy chain comprising the amino acid sequence of SEQ ID NO: 68 paired with a common light chain comprising the amino acid sequence of SEQ ID NO: 70, and a second heavy chain comprising the amino acid sequence of SEQ ID NO: 66 paired with a common light chain comprising the amino acid sequence of SEQ ID NO: 70.

In some aspects the disclosure provides a bispecific antibody comprising a first antigen-binding domain that binds specifically to human CD28 and a second antigen-binding domain that binds specifically to human PD-L1, wherein the bispecific antibody comprises a first heavy chain comprising the amino acid sequence of SEQ ID NO: 40 paired with a common light chain comprising the amino acid sequence of SEQ ID NO: 30, and a second heavy chain comprising the amino acid sequence of SEQ ID NO: 26 paired with a common light chain comprising the amino acid sequence of SEQ ID NO: 30.

In some aspects the disclosure provides a bispecific antibody comprising a first antigen-binding domain that binds specifically to human CD28 and a second antigen-binding domain that binds specifically to human PD-L1, wherein the bispecific antibody comprises a first heavy chain comprising the amino acid sequence of SEQ ID NO: 28 paired with a common light chain comprising the amino acid sequence of SEQ ID NO: 30, and a second heavy chain comprising the amino acid sequence of SEQ ID NO: 26 paired with a common light chain comprising the amino acid sequence of SEQ ID NO: 30.

In some embodiments, the bispecific antibody is a human antibody.

In certain aspects, the present disclosure provides a pharmaceutical composition comprising the bispecific antigen-binding molecule disclosed herein, and a pharmaceutically acceptable carrier or diluent. In certain other aspects, the present disclosure provides a pharmaceutical composition comprising the bispecific antibody disclosed herein, and a pharmaceutically acceptable carrier or diluent.

The present disclosure also provides a method for making a bi-specific antigen-binding protein set forth herein comprising: (a) introducing one or more nucleic acid molecules comprising nucleic acid sequences encoding the immunoglobulin chains of said bispecific antigen-binding protein into a host cell (e.g., a CHO cell); (b) culturing the host cell under conditions favorable to expression of the nucleic acid molecules; and (c) optionally, isolating the antigen-binding protein or immunoglobulin chain from the host cell and/or medium in which the host cell is grown. Any antigen-binding protein or immunoglobulin chain which is a product of such a method is part of the present disclosure.

In some embodiments, the host cell is a Chinese hamster ovary (CHO) cell.

In some embodiments, the method further comprises formulating the antigen-binding molecule as a pharmaceutical composition comprising an acceptable carrier.

An antigen-binding molecule or immunoglobulin chain which is a product of the method set forth herein is also part of the present disclosure.

The present disclosure also provides a nucleic acid molecule comprising a nucleotide sequence encoding a bispecific antigen-binding molecule as set forth herein, or a set of nucleic acid molecules comprising nucleotide sequences encoding the HCVR of the first antigen-binding domain that specifically binds to human CD28, the HCVR of the second antigen-binding domain that specifically binds to human PD-L1, and the LCVR of an isolated bispecific antigen-binding molecule set forth herein. Expression vectors comprising the nucleic acid molecule, or a set of expression vectors comprising the set of nucleic acid molecules of the present disclosure are also part of the present disclosure, as well as host cells (e.g., CHO cell) comprising a nucleic acid molecule, vector or antigen-binding protein of the present disclosure.

The present disclosure provides a method of producing a bispecific antigen-binding molecule that binds to PD-L1 and CD28 comprising: (a) culturing the host cell set forth herein under conditions favorable for production of the bispecific antigen-binding molecule; and (b) optionally, isolating the antigen-binding molecule or immunoglobulin chain from the host cell and/or medium in which the host cell is grown. In some embodiments, the host cell is a CHO cell. In some embodiments, the method further comprises formulating the antigen-binding molecule as a pharmaceutical composition comprising an acceptable carrier.

Antigen-binding molecule or immunoglobulin chain which is a product of the method disclosure herein is also part of the present disclosure.

The present disclosure also provides a nucleic acid molecule comprising a nucleotide sequence encoding a bispecific antibody as set forth herein, or a set of nucleic acid molecules comprising nucleotide sequences encoding the heavy chain of the first antigen-binding domain that specifically binds to human CD28, the heavy of the second antigen-binding domain that specifically binds to human PD-L1, and the light chain of a bispecific antibody as set forth herein. Expression vectors comprising the nucleic acid of the present disclosure are also part of the present disclosure, as well as host cells (e.g., CHO cell) comprising a nucleic acid molecule, vector or antibody of the present disclosure.

The present disclosure provides a method of producing a bispecific antibody that binds to PD-L1 and CD28 comprising: (a) culturing the host cell set forth herein under conditions favorable for production of the bispecific antigen-binding molecule; and (b) optionally, isolating the antigen-binding molecule or immunoglobulin chain from the host cell and/or medium in which the host cell is grown. In some embodiments, the host cell is a CHO cell. In some embodiments, the method further comprises formulating the bispecific antibody as a pharmaceutical composition comprising an acceptable carrier.

An antibody which is a product of the method disclosure herein is also part of the present disclosure.

The present disclosure also provides a method for treating a hyperproliferative disease (e.g., cancer), in a subject (e.g., a human) in need thereof, comprising administering (e.g., subcutaneously, intravenously or intramuscularly) an effective amount of bispecific antigen-binding protein or composition or formulation. In an embodiment of the disclosure, cancer is a B cell cancer, basal cell carcinoma, bladder urothelial carcinoma, brain cancer, breast cancer, cervical cancer, cervical squamous cell carcinoma, colorectal cancer, diffuse large B cell lymphoma, endometrial adenocarcinoma, endometrial cancer, esophageal carcinoma, gastroesophageal adenocarcinoma, gastroesophageal cancer, glioblastoma multiforme, head & neck squamous cell carcinoma, hepatocellular carcinoma, lung adenocarcinoma, lung cancer, lung squamous cell carcinoma, melanoma, multiple myeloma, non-small cell lung cancer, ovarian cancer, pancreatic cancer, prostate cancer, rectal cancer, skin cancer, or a T cell cancer. In one embodiment, the cancer comprises PD-L1 expressing cancer cells.

In some aspects, the present disclosure provides a method of inhibiting growth of a tumor in a subject, comprising administering an isolated bispecific antigen-binding molecule, or a bispecific antibody, or a pharmaceutical composition as set forth herein to the subject.

In some embodiments, the tumor is a B cell cancer, basal cell carcinoma, bladder urothelial carcinoma, brain cancer, breast cancer, cervical cancer, cervical squamous cell carcinoma, colorectal cancer, diffuse large B cell lymphoma, endometrial adenocarcinoma, endometrial cancer, esophageal carcinoma, gastroesophageal adenocarcinoma, gastroesophageal cancer, glioblastoma multiforme, head & neck squamous cell carcinoma, hepatocellular carcinoma, lung adenocarcinoma, lung cancer, lung squamous cell carcinoma, melanoma, multiple myeloma, non-small cell lung cancer, ovarian cancer, pancreatic cancer, prostate cancer, rectal cancer, skin cancer, or a T cell cancer.

In some embodiments, the tumor expresses PD-L1.

In some embodiments, the method further comprises administering a second therapeutic agent or therapeutic regimen.

In some embodiments, the second therapeutic agent or therapeutic regimen comprises a chemotherapeutic drug, a DNA alkylator, an immunomodulator, a proteasome inhibitor, a histone deacetylase inhibitor, radiotherapy, surgery, a stem cell transplant, a bispecific antibody that interacts with a tumor associated antigen (TAA) and a T cell or immune cell antigen, an antibody drug conjugate, an oncolytic virus, a bispecific antibody conjugated to an anti-tumor agent, a VEGF inhibitor, a checkpoint inhibitor, a GITR agonist, a CD27 agonist, a 4-1 BB activator, a PD-1 inhibitor, a CTLA-4 inhibitor, an EGFR inhibitor, Ang2 inhibitor, a MUC16 inhibitor, a cancer vaccine, a cytokine, a modified IL2, a modified IL12, IL4 inhibitor, IL6 inhibitor, a corticosteroid, or combinations thereof.

In some embodiments, the T cell or immune cell antigen is CD3.

In some embodiments, the TAA is selected from the group consisting of AFP, ALK, BAGE proteins, BCMA, BIRC5 (survivin), BIRC7, β-catenin, brc-abl, BRCA1, BORIS, CA9, carbonic anhydrase IX, caspase-8, CALR, CCR5, CD19, CD20 (MS4A1), CD22, CD30, CD40, CDK4, CEA, CTLA4, cyclin-B1, CYP1B1, EGFR, EGFRvIII, ErbB2/Her2, ErbB3, ErbB4, ETV6-AML, EpCAM, EphA2, Fra-1, FOLR1, GAGE proteins (e.g., GAGE-1, -2), GD2, GD3, GloboH, glypican-3, GM3, gp100, Her2, HLA/B-raf, HLA/k-ras, HLA/MAGE-A3, hTERT, LMP2, MAGE proteins (e.g., MAGE-1, -2, -3, -4, -6, and -12), MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, Ras, RGS5, Rho, SART-1, SART-3, STEAP1, STEAP2, TAG-72, TGF-β, TMPRSS2, Thompson-nouvelle antigen (Tn), TRP-1, TRP-2, tyrosinase, and uroplakin-3.

In other aspects, the present disclosure provides use of the bispecific antigen-binding molecule, or a bispecific antibody, or a pharmaceutical composition as set forth herein in the treatment of a tumor.

In some embodiments, the tumor is a B cell cancer, basal cell carcinoma, bladder urothelial carcinoma, brain cancer, breast cancer, cervical cancer, cervical squamous cell carcinoma, colon cancer, colorectal cancer, diffuse large B cell lymphoma, endometrial adenocarcinoma, endometrial cancer, esophageal carcinoma, gastroesophageal adenocarcinoma, gastroesophageal cancer, glioblastoma multiforme, head & neck squamous cell carcinoma, hepatocellular carcinoma, melanoma, multiple myeloma, leukemia, lung adenocarcinoma, lung cancer, lung squamous cell carcinoma, non-small cell lung cancer, ovarian cancer, pancreatic cancer, prostate cancer, rectal cancer, skin cancer, or a T cell cancer.

In some embodiments, the tumor expresses PD-L1.

In some embodiments, the antigen-binding molecule or pharmaceutical composition is for use in combination with a second therapeutic agent or therapeutic regimen that comprises a chemotherapeutic drug, a DNA alkylator, an immunomodulator, a proteasome inhibitor, a histone deacetylase inhibitor, radiotherapy, surgery, a stem cell transplant, a bispecific antibody that interacts with a tumor associated antigen (TAA) and a T cell or immune cell antigen, an antibody drug conjugate, an oncolytic virus, a bispecific antibody conjugated to an anti-tumor agent, a VEGF inhibitor, a checkpoint inhibitor, a GITR agonist, a CD27 agonist, a 4-1 BB activator, a PD-1 inhibitor, a CTLA-4 inhibitor, an EGFR inhibitor, Ang2 inhibitor, a MUC16 inhibitor, a cancer vaccine, a cytokine, a modified IL2, a modified IL12, IL4 inhibitor, IL6 inhibitor, a corticosteroid, or combinations thereof.

### BRIEF DESCRIPTION OF THE FIGURES

FIGS. 1A-1B relate to Example 7. FIG. 1A is a graph showing average tumor volume in mice administered the indicated antibodies. FIG. 1B is a graph showing the probability of survival of mice administered the indicated antibodies

FIG. 2 relates to Example 8 and is a graph showing average tumor volume in mice administered the indicated antibodies.

FIGS. 3A-3E relate to Example 8 and are graphs showing tumor volume in individual mice administered specific antibodies. The fraction of mice that were tumor free (TF) is indicated. FIG. 3A shows tumor volume in individual mice administered isotype-control antibody.

FIG. 3B shows tumor volume in individual mice administered REGN6192, lot 2. FIG. 3C shows tumor volume in individual mice administered REGN6193, lot 2. FIG. 3D shows tumor volume in individual mice administered REGN6194. FIG. 3E shows tumor volume in individual mice administered REGN6194 and cemiplimab.

FIG. 4 relates to Example 8 and is a graph showing the probability of survival of mice administered the indicated antibodies.

FIGS. 5A-5B relate to Example 9. FIG. 5A is a graph showing average tumor volume in mice administered the indicated antibodies. FIG. 5B is a graph showing the probability of survival of mice administered the indicated antibodies.

FIGS. 6A-6C relate to Example 10 and are graphs showing the concentration of specific cytokines in the blood of mice administered the indicated antibodies. FIG. 6A shows the concentration of IL-2. FIG. 6B shows the concentration of IL-5. FIG. 6C shows the concentration of IL-4. TGN1412 is an anti-CD28 superagonist. Suntharalingam, N. Engl. J. Med., 355(10):1018-1028 (2006).

FIG. 7 relates to Example 11 and is a graph showing average tumor volume in mice administered the indicated antibodies.

FIGS. 8A-8E relate to Example 12. FIGS. 8A-8D are graphs showing average tumor volume in mice administered the indicated antibodies and engrafted with tumor cells with specific ratios of parental M38 cells to human PD-L1+M38 cells. FIG. 8A shows average tumor volume of mice engrafted with a ratio of 0:100. FIG. 8B shows average tumor volume of mice engrafted with a ratio of 50:50. FIG. 8C shows average tumor volume of mice engrafted with a ratio of 90:10. FIG. 8D shows average tumor volume of mice engrafted with a ratio of 99:1. FIG. 8E is a graph showing the probability of survival of mice administered the indicated antibodies and engrafted with tumor cells with the indicated ratios of parental M38 cells to human PD-L1+M38 cells.

FIGS. 9A-9E relate to Example 13. FIG. 9A is a graph showing average tumor volume in mice administered the indicated antibodies. FIGS. 9B-9E are graphs showing tumor volume in individual mice administered specific antibodies. The fraction of mice that were tumor free (TF) is indicated. FIG. 9B shows tumor volume in individual mice administered isotype-control antibody. FIG. 9C shows tumor volume in individual mice administered REGN6194. FIG. 9D shows tumor volume in individual mice administered cemiplimab. FIG. 9E shows tumor volume in individual mice administered REGN6194 and cemiplimab.

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 1 01 and all values in between (e.g., 99.1, 99.2, 99.3, 99.4, etc.).

Although any methods and materials similar or equivalent to those described herein can be used in the practice or testing of the present disclosure, the preferred methods and materials are now described.

Checkpoint inhibition with PD-L1 blocking mAb are known to release the break on T cell activation, but their efficacy as a single agent is often not always sufficient to achieve tumor clearance and a durable anti-tumor response in many cancers. Several approaches to improve the response rate to PD-1/PD-L1 inhibition are currently being evaluated, e.g., identification of biomarkers to predict responsiveness to PD-L1 mAbs, or combination with chemotherapy or radiotherapy. The challenge however is that many of these combinations are often based on the availability of pre-existing drug and a post-hoc rationale to combine therapies, rather than a truly hypothesis-driven approach, which in some cases has led to worse outcomes for the patient. It is evident that checkpoint inhibition and reactivation of the immune system offers the potential of long term remission in a number of patients, therefore methods to further improve or enhance T cell activity to promote a more durable responses are warranted.

The present disclosure shows the potential benefit of combining PD-L1 inhibition with CD28-agonism (which provides “signal 2”) to enhance anti-tumor activity. The PD-L1 binding arm of the bispecific antibody acts like a bridge to anchor CD28-activating domain of the antibody to tumor, providing a pan-tumor costimulatory approach (tumor agnostic) to convert an inhibitory checkpoint (PD-L1) widely present across various tumor indications and turn it into a costimulatory signal to activate CD28 on T cells.

CAR-T approaches have also employed chimeric receptors that artificially activate both “signal 1” and “signal 2” to provide improved anti-tumor activity. The bispecific antigen-binding molecules of the present disclosure provide practical benefits over CAR-T therapies in that it does not require a laborious cell therapy preparation that must be individually customized for each patient, nor does it require that patients be pre-emptively “lymphodepleted” via toxic chemotherapy that is often associated with adverse effects so that they can't accept cell therapy. This bispecific approach offers the potential for increased efficacy as well as increased safety through its specificity of action. Collectively, the data here suggest that the bispecific PD-L1×CD28 antigen-binding molecules of the present disclosure may provide well-tolerated, biologics solutions with markedly enhanced and synergistic anti-tumor activity, when used as monotherapy or in combination with a PD-1 inhibitory antibody (e.g., cemiplimab) and/or a TAA×CD3 bispecific antibody.

### Definitions

“PD-L1” and “PD-L1 fragment,” as used herein refer to the human PD-L1 protein (also known as CD274, B7-H, B7H1, PDCD1L1, and PDCD1LG1) or a fragment thereof unless specified as being from a non-human species (e.g., “mouse PD-L1,” “mouse PD-L1 fragment,” “monkey PD-L1,” “monkey PD-L1 fragment,” etc.). In an embodiment of the disclosure, human PD-L1 comprises the amino acid sequence set forth in NCBI accession no. AAH69381.1. In one embodiment, a human PD-L1 fragment is shown with a C-terminal myc-myc-hexahistidine tag (hPD-L1.mmH) (SEQ ID NO: 71), or with human or mouse Fc (SEQ ID NOs: 73 and 74).

“CD28,” as used herein, refers to the human CD28 protein which is expressed on T cells as a costimulatory receptor unless specified as being from a non-human species. In an embodiment of the disclosure, human CD28 comprises the amino acid sequence as set forth in NCBI accession No. NP_006130.1. In one embodiment, human CD28 is expressed with a C-terminal murine Fc tag (hCD28.mFc, SEQ ID NO: 72)

“Isolated” antigen-binding proteins (e.g., antibodies or antigen-binding fragments thereof), polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antigen-binding proteins (e.g., antibodies or antigen-binding fragments).

The following references relate to BLAST algorithms often used for sequence analysis: BLAST ALGORITHMS: Altschul et al. (2005) FEBS J. 272(20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

An “antibody” is an immunoglobulin molecule comprising four polypeptide chains, two heavy chains (HC) and two light chains (LC) inter-connected by disulfide bonds. Each heavy chain (HC) comprises a heavy chain variable region (abbreviated herein as HCVR or V_{H}) and a heavy chain constant region (e.g., IgG, IgG1 or IgG4). The heavy chain constant region comprises three domains, C_{H}1, C_{H}2 and C_{H}3. Each light chain (LC) comprises a light chain variable region (abbreviated herein as LCVR or V_{L}) and a light chain constant region (e.g., lambda or kappa). The light chain constant region comprises one domain (C_{L}1). The V_{H} and V_{L}regions can be further subdivided into regions of hypervariability, termed complementarity determining regions (CDRs), interspersed with regions that are more conserved, termed framework regions (FR). Each V_{H} and V_{L} includes three CDRs and four FRs, arranged from amino-terminus to carboxy-terminus in the following order: FR1, CDR1, FR2, CDR2, FR3, CDR3, FR4. A heavy chain CDR may be referred to as HCDR and a light chain CDR may be referred to as LCDR. In different embodiments, the FRs of an antibody (or antigen-binding portion thereof) may be identical to the human germline sequences, or may be naturally or artificially modified.

An antigen-binding arm of a Y-shaped IgG antibody (e.g., a CD28 or PD-L1 binding arm) refers to a structural portion of the antibody that confers binding specificity to the antigen. For example, an antigen-binding arm of an IgG antibody has a heavy chain (HC) associated with a light chain (LC).

An antibody which, for example, is bispecific includes an arm (or domain) that binds to a first antigen and another arm (or domain) that binds to a second antigen. For example, an PD-L1×CD28 bispecific antibody includes one arm that binds PD-L1 and another arm that binds to CD28.

Bispecific antigen-binding molecules (e.g., bispecific antibodies) may have an effector arm that binds to a first antigen and a targeting arm that binds to second antigen. The effector arm may be the first antigen-binding domain (e.g., anti-CD28) that binds to the antigens on effector cells (e.g., T cells). The targeting arm may be the second antigen-binding domain (e.g., anti-PD-L1 antibody) that binds to the antigens on target cells (e.g., tumor cells or immune cells). In the context of the present disclosure, the effector arm binds to CD28 and the targeting arm binds to the inhibitory checkpoint PD-L1.

An “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. A multispecific antigen-binding fragment of an antibody binds to multiple antigens (e.g., two different antigens if the fragment is bispecific). Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Non-limiting examples of antigen-binding fragments include: (i) Fab fragments; (ii) F(ab′)₂ fragments; (iii) Fd fragments; (iv) Fv fragments; (v) single-chain Fv (scFv) molecules; and (vi) dAb fragments.

An antigen-binding fragment of an antibody will, in an embodiment, comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2—C_{H}3; (vi) V_{H}-C_{H}2—C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2—C_{H}3; (xiii) V_{L}-C_{H}2-C_{H}3; and (Xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The term “recombinant” antigen-binding proteins, such as antibodies or antigen-binding fragments thereof, refers to such molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a host cell (e.g., Chinese hamster ovary (CHO) cell) or cellular expression system or isolated from a recombinant combinatorial human antibody library. The present disclosure includes recombinant antigen-binding proteins as set forth herein.

The term “specifically binds” or “binds specifically” refers to those antigen-binding proteins (e.g., antibodies or antigen-binding fragments thereof) having a binding affinity to an antigen, such as PD-L1 or CD28 protein, expressed as KD, of less than about 10⁻⁶ M (e.g., 10⁻⁷ M, 10⁻⁸ M, 10⁻⁹ M, 10⁻¹⁰M, 10⁻¹¹ M or 10⁻¹² M), as measured by real-time, label free bio-layer interferometry assay, for example, at 25° C. or 37° C., e.g., an Octet® HTX biosensor, or by surface plasmon resonance, e.g., BIACORE™, or by solution-affinity ELISA. “Anti-PD-L1” refers to an antigen-binding protein (or other molecule such as an antigen-binding arm), for example an antibody or antigen-binding fragment thereof, that binds specifically to PD-L1 and “anti-CD28” refers to an antigen-binding protein (or other molecule such as an antigen-binding arm), for example an antibody or antigen-binding fragment thereof, that binds specifically to CD28. “PD-L1×CD28” refers to refers to an antigen-binding protein (or other molecule), for example an antibody or antigen-binding fragment thereof, that binds specifically to PD-L1 and to CD28 (and, optionally, to one or more other antigens).

The present disclosure includes antigen-binding proteins, e.g., antibodies or antigen-binding fragments, that bind to the same PD-L1 and CD28 epitopes as an antigen-binding protein of the present disclosure.

The term “epitope” refers to an antigenic determinant (e.g., on PD-L1 or CD28) that interacts with a specific antigen-binding site of an antigen-binding protein, e.g., a variable region of an antibody molecule, known as a paratope. A single antigen may have more than one epitope. Thus, different antibodies may bind to different areas on an antigen and may have different biological effects. The term “epitope” may also refer to a site on an antigen to which B and/or T cells respond and/or to a region of an antigen that is bound by an antibody. Epitopes may be defined as structural or functional. Functional epitopes are generally a subset of the structural epitopes and have those residues that directly contribute to the affinity of the interaction. Epitopes may be linear or conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

Methods for determining the epitope of an antigen-binding protein, e.g., antibody or fragment or polypeptide, include alanine scanning mutational analysis, peptide blot analysis (Reineke (2004) Methods Mol. Biol. 248: 443-63), peptide cleavage analysis, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antigen-binding protein (e.g., antibody or fragment or polypeptide) interacts is hydrogen/deuterium exchange detected by mass spectrometry. See, e.g., Ehring (1999) Analytical Biochemistry 267: 252-259; Engen and Smith (2001) Anal. Chem. 73: 256A-265A.

The present disclosure includes antigen-binding proteins that compete for binding to CD28 and PD-L1 with an antigen-binding protein of the present disclosure. The term “competes” as used herein, refers to an antigen-binding protein (e.g., antibody or antigen-binding fragment thereof) that binds to an antigen and inhibits or blocks the binding of another antigen-binding protein (e.g., antibody or antigen-binding fragment thereof) to the antigen. Unless otherwise stated, the term also includes competition between two antigen-binding proteins e.g., antibodies, in both orientations, i.e., a first antibody that binds antigen and blocks binding by a second antibody and vice versa. Thus, in an embodiment of the disclosure, competition occurs in one such orientation. In certain embodiments, the first antigen-binding protein (e.g., antibody) and second antigen-binding protein (e.g., antibody) may bind to the same epitope. Alternatively, the first and second antigen-binding proteins (e.g., antibodies) may bind to different, but, for example, overlapping or non-overlapping epitopes, wherein binding of one inhibits or blocks the binding of the second antibody, e.g., via steric hindrance. Competition between antigen-binding proteins (e.g., antibodies) may be measured by methods known in the art, for example, by a real-time, label-free bio-layer interferometry assay. Also, binding competition between antigen-binding proteins (e.g., monoclonal antibodies (mAbs)) can be determined using a real time, label-free bio-layer interferometry assay on an Octet RED384 biosensor (Pall ForteBio Corp.).

Typically, an antibody or antigen-binding fragment of the disclosure which is modified in some way retains the ability to specifically bind to PD-L1 and CD28, e.g., retains at least 10% of its PD-L1 and CD28 binding activity (when compared to the parental antibody) when that activity is expressed on a molar basis. Preferably, an antibody or antigen-binding fragment of the disclosure retains at least 20%, 50%, 70%, 80%, 90%, 95% or 100% or more of the PD-L1 and CD28 binding affinity as the parental antibody. It is also intended that an antibody or antigen-binding fragment of the disclosure may include conservative or non-conservative amino acid substitutions (referred to as “conservative variants” or “function conserved variants” of the antibody) that do not substantially alter its biologic activity.

A “variant” of a polypeptide, such as an immunoglobulin chain V_{H}, V_{L}, HC or LC or CDR thereof comprising the amino acid sequence specifically set forth herein), refers to a polypeptide comprising an amino acid sequence that is at least about 70-99.9% (e.g., at least 70, 72, 74, 75, 76, 79, 80, 81, 82, 83, 84, 85, 86, 87, 88, 89, 90, 91, 92, 93, 94, 95, 96, 97, 98, 99, 99.5 or 99.9%) identical or similar to a referenced amino acid sequence that is set forth herein (e.g., any of SEQ ID NOs: 2, 4, 6; 8; 10; 12; 14; 16; 18; 20; 22; 24; 26; 28; 30; 32; 34; 36; 38; 40; 42; 44; 46; 48; 50; 52; 54; 56; 58; 60; 62; 64; 66; 68 or 70); when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment).

Moreover, a variant of a polypeptide may include a polypeptide such as an immunoglobulin chain V_{H}, V_{L}, HC or LC or CDR thereof) which may include the amino acid sequence of the reference polypeptide whose amino acid sequence is specifically set forth herein but for one or more (e.g., 1, 2, 3, 4, 5, 6, 7, 8, 9 or 10) mutations, e.g., one or more missense mutations (e.g., conservative substitutions), non-sense mutations, deletions, or insertions. For example, the present disclosure includes CD28×PD-L1 antigen-binding proteins which include an PD-L1 binding arm immunoglobulin light chain (or V_{L}) variant comprising the amino acid sequence set forth in SEQ ID NO: 18 but having one or more of such mutations and/or an immunoglobulin heavy chain (or V_{H}) variant comprising the amino acid sequence set forth in SEQ ID NO: 2 but having one or more of such mutations. In an embodiment of the disclosure, a CD28×PD-L1 antigen-binding protein includes an immunoglobulin light chain variant comprising LCDR1, LCDR2 and LCDR3 wherein one or more (e.g., 1 or 2 or 3) of such CDRs has one or more of such mutations (e.g., conservative substitutions) and/or an immunoglobulin heavy chain variant comprising HCDR1, HCDR2 and HCDR3 wherein one or more (e.g., 1 or 2 or 3) of such CDRs has one or more of such mutations (e.g., conservative substitutions).

A “conservatively modified variant” or a “conservative substitution”, e.g., of an immunoglobulin chain set forth herein, refers to a variant wherein there is one or more substitutions of amino acids in a polypeptide with other amino acids having similar characteristics (e.g., charge, side-chain size, hydrophobicity/hydrophilicity, backbone conformation and rigidity, etc.). Such changes can frequently be made without significantly disrupting the biological activity of the antibody or fragment. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4^{th} Ed.)). In addition, substitutions of structurally or functionally similar amino acids are less likely to significantly disrupt biological activity. The present disclosure includes PD-L1×CD28 antigen-binding proteins and/or binding arms comprising such conservatively modified variant immunoglobulin chains.

Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Alternatively, a conservative replacement is any change having a positive value in the PAM250 log-likelihood matrix disclosed in Gonnet et al. (1992) Science 256: 1443-45.

As used herein, the singular forms “a,” “an,” and “the” include plural reference unless the context clearly dictates otherwise. As used herein, the terms “including,” “comprising,” “containing,” or “having” and variations thereof are meant to encompass the items listed thereafter and equivalents thereof as well as additional subject matter unless otherwise noted. As used herein, the phrases “in one embodiment,” “in various embodiments,” “in some embodiments,” and the like are used repeatedly. Such phrases do not necessarily refer to the same embodiment, but they may unless the context dictates otherwise. As used herein, the terms “and/or” or “/” means any one of the items, any combination of the items, or all of the items with which this term is associated.

### PD-L1×CD28 Antigen-Binding Molecules, Antibodies or Antigen-Binding Fragments Thereof

The antibodies (or antigen-binding molecules) of the present disclosure may be bi-specific, or multispecific. Multispecific antibodies or antigen-binding molecules may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. The antibodies of the present disclosure can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second binding specificity.

Use of the expression “anti-CD28 antibody” herein is intended to include both monospecific anti-CD28 antibodies as well as multispecific (e.g., bispecific) antibodies or antigen-binding molecules comprising a CD28-binding arm and a second arm that binds PD-L1. Thus, the present disclosure includes bispecific antibodies wherein one arm of an immunoglobulin binds human CD28, and the other arm of the immunoglobulin is specific for PD-L1. The CD28-binding arm can comprise any of the HCVR/LCVR or CDR amino acid sequences as set forth in Tables 3 and 8 herein. In certain embodiments, the CD28-binding arm binds human CD28 and induces human T-cell proliferation.

According to certain exemplary embodiments, the present disclosure includes bispecific antigen-binding molecules that specifically bind CD28 and PD-L1. Such molecules may be referred to herein as, e.g., “anti-CD28/anti-PD-L1,” or “anti-CD28×PD-L1,” or “CD28×PD-L1” or “PD-L1×CD28”, or “anti-PD-L1/anti-CD28,” or “anti-PD-L1×CD28,” or “PD-L1×CD28” bispecific molecules, or “anti-PD-L1×anti-CD28” or “anti-CD28×anti-PD-L1”, or other similar terminology.

According to certain exemplary embodiments, the bispecific antigen-binding molecules (e.g., bispecific antibody) may have an effector arm and a targeting arm. The effector arm may be the first antigen-binding domain that binds to CD28 on effector cells (e.g., T cells). The targeting arm may be the second antigen-binding domain that binds to an antigen (e.g., an immune checkpoint) on target cells (e.g., tumor cells or antigen-presenting cells). In the context of the present disclosure, the effector arm binds to CD28 and the targeting arm binds to the inhibitory checkpoint ligand PD-L1. The bispecific anti-CD28/PD-L1 may provide a pan-tumor costim approach (tumor agnostic) to convert an inhibitory checkpoint (PD-L1) widely present across various tumor indications and turn it into a costimulatory signal to activate CD28 on T cells.

As used herein, the expression “antigen-binding molecule” means a protein, polypeptide or molecular complex comprising or consisting of at least one complementarity determining region (CDR) that alone, or in combination with one or more additional CDRs and/or framework regions (FRs), specifically binds to a particular antigen. In certain embodiments, an antigen-binding molecule is an antibody or a fragment of an antibody, as those terms are defined elsewhere herein.

As used herein, the expression “bispecific antigen-binding molecule” means a protein, polypeptide or molecular complex (e.g., an antibody or antigen-binding fragment thereof) comprising at least a first antigen-binding domain and a second antigen-binding domain. Each antigen-binding domain within the bispecific antigen-binding molecule comprises at least one CDR that alone, or in combination with one or more additional CDRs and/or FRs, specifically binds to a particular antigen. In the context of the present disclosure, the first antigen-binding domain specifically binds a first antigen (e.g., CD28), and the second antigen-binding domain specifically binds a second, distinct antigen (e.g., PD-L1).

In certain exemplary embodiments, the bispecific antigen-binding molecule is a bispecific antibody. Each antigen-binding domain of a bispecific antibody comprises a heavy chain variable domain (HCVR) and a light chain variable domain (LCVR).

The first antigen-binding domain and the second antigen-binding domain may be directly or indirectly connected to one another to form a bispecific antigen-binding molecule of the present disclosure. Alternatively, the first antigen-binding domain and the second antigen-binding domain may each be connected to a separate multimerizing domain. The association of one multimerizing domain with another multimerizing domain facilitates the association between the two antigen-binding domains, thereby forming a bispecific antigen-binding molecule. As used herein, a “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin C_{H}3 domain. A non-limiting example of a multimerizing component is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group. The Fc domain may comprise wild-type or modified IgG isotype.

Bispecific antigen-binding molecules of the present disclosure will typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In certain embodiments, the multimerizing domain is an Fc fragment or an amino acid sequence of 1 to about 200 amino acids in length containing at least one cysteine residues. In other embodiments, the multimerizing domain is a cysteine residue, or a short cysteine containing peptide. Other multimerizing domains include peptides or polypeptides comprising or consisting of a leucine zipper, a helix-loop motif, or a coiled-coil motif.

Any bispecific antibody format or technology may be used to make the bispecific antigen-binding molecules of the present disclosure. For example, an antibody or antigen-binding fragment thereof having a first antigen-binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context of the present disclosure include, without limitation, e.g., scFv-based or diabody bispecific formats, IgG-scFv fusions, dual variable domain (OVO)-Ig, Quadroma, knobs-into-holes, common light chain (e.g., common light chain with knobs-intoholes, etc.), CrossMab, CrossFab, (SEEO)body, leucine zipper, Ouobody, IgG1/IgG2, dual acting Fab (OAF)-IgG, and Mab² bispecific formats (see, e.g., Klein et al. 2012, mAbs 4:6, 1-11, and references cited therein, for a review of the foregoing formats).

In the context of bispecific antigen-binding molecules of the present disclosure, the multimerizing domains, e.g., Fc domains, may comprise one or more amino acid changes (e.g., insertions, deletions or substitutions) as compared to the wild-type, naturally occurring version of the Fc domain. For example, the disclosure includes bispecific antigen-binding molecules comprising one or more modifications in the Fc domain that results in a modified Fc domain having a modified binding interaction (e.g., enhanced or diminished) between Fc and FcRn. In one embodiment, the bispecific antigen-binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications include, e.g., a modification at position 250 (e.g., E or Q); 250 and 428 (e.g., L or F); 252 (e.g., LN/FIW or T), 254 (e.g., S or T), and 256 (e.g., S/R/Q/EID or T); or a modification at position 428 and/or 433 (e.g., UR/S/P/Q or K) and/or 434 (e.g., H/F or V); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252,254, and 256 (e.g., 252Y, 254T, and 256E) modification; a 2500 and 428L modification (e.g., T250Q and M428L); and a 307 and/or 308 modification (e.g., 308F or 308P).

The present disclosure also includes bispecific antigen-binding molecules comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L 18M, N44S, K52N, V57M, and V821 (by IMGT; D356E, L358M, N384S, K392N, V397M, and V4221 by EU) in the case of IgG1 antibodies; N44S, K52N, and V821 (IMGT; N384S, K392N, and V4221 by EU) in the case of IgG2 antibodies; and Q15R, N44S, K52N, V57M, R69K, E79Q, and V821 (by IMGT; Q355R, N384S, K392N, V397M, R409K, E419Q, and V4221 by EU) in the case of IgG4 antibodies.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 C_{H}1]—[IgG4 upper hinge]—[IgG2 lower hinge]—[IgG4 C_{H}2]—[IgG4 C_{H}3]. Another example of a chimeric Fc domain that can be included in any of the antigen-binding molecules set forth herein comprises, from N- to C-terminus: [IgG1 C_{H}1]—[IgG1 upper hinge]—[IgG2 lower hinge]—[IgG4 C_{H}2]—[IgG1 C_{H}3]. These and other examples of chimeric Fc domains that can be included in any of the antigen-binding molecules of the present disclosure are described in WO2014/022540 A1, Chimeric Fc domains having these general structural arrangements, and variants thereof, can have altered Fc receptor binding, which in turn affects Fc effector function.

Antibodies and antigen-binding fragments of the present disclosure comprise immunoglobulin chains including the amino acid sequences specifically set forth herein (and variants thereof) as well as cellular and in vitro post-translational modifications to the antibody or fragment. For example, the present disclosure includes antibodies and antigen-binding fragments thereof that specifically bind to PD-L1 and CD28 comprising heavy and/or light chain amino acid sequences set forth herein as well as antibodies and fragments wherein one or more asparagine, serine and/or threonine residues is glycosylated, one or more asparagine residues is deamidated, one or more residues (e.g., Met, Trp and/or His) is oxidized, the N-terminal glutamine is pyroglutamate (pyroE) and/or the C-terminal lysine or other amino acid is missing.

The bispecific antigen-binding molecules of the present disclosure comprise a first antigen-binding arm that specifically binds CD28 (“CD28-binding arm” “CD28 binding domain”). In certain embodiments, the CD28-binding arm comprises HCVR and LCVR comprising amino acid sequences as disclosed herein. The bispecific antigen-binding molecules also comprise a second antigen-binding arm that specifically binds PD-L1 (“an PD-L1 binding arm” or “PD-L1 binding domain”). In certain embodiments, the PD-L1-binding arm comprises HCVR and LCVR comprising amino acid sequences as disclosed herein. In certain embodiments, the PD-L1-binding arm includes a heavy chain immunoglobulin that comprises a V_{H} including the combination of heavy chain CDRs (HCDR1, HCDR2 and HCDR3) and the corresponding light chain immunoglobulin that comprises a V_{L} including the combination of light chain CDRs (LCDR1, LCDR2 and LCDR3) which are set forth herein or in International patent application publication no. WO2014/004427.

### Polynucleotides and Methods of Making

An isolated polynucleotide molecule or a set of polynucleotide molecules comprising polyneucleotide sequences encoding the immunoglobulin chains of any PD-L1×CD28 multispecific antigen-binding protein set forth herein forms part of the present disclosure. The present disclosure also includes a vector or a set of vectors comprising the polynucleotide molecules and/or a host cell (e.g., Chinese hamster ovary (CHO) cell) comprising the polynucleotide molecules, vector(s) or antigen-binding protein set forth herein.

A polynucleotide molecule or sequence refers to DNA or RNA. The present disclosure includes any polynucleotide of the present disclosure, for example, encoding an immunoglobulin V_{H}, V_{L}, CDR-H, CDR-L, HC or LC of an PD-L1 Binding Arm and/or a CD28 Binding Arm, optionally, which is operably linked to a promoter or other expression control sequence. For example, the present disclosure provides any polynucleotide molecule or set of polynucleotide molecules (e.g., DNA) that includes a nucleotide sequence set forth in Table 2 and a nucleotide sequence set forth in Table 4.

The present disclosure includes a polynucleotide comprising a nucleotide sequence set forth in SEQ ID NO: 1, 3, 5, 7, 9, 11, 13 15, 17, 19, 21, 29, 31, 33, 35, 39, 41, 43, 45, 49, 51, 53, 55, 57, 59, 61, 63, 65, 67 and/or 69, optionally operably linked to a promoter or other expression control sequence or other polynucleotide sequence.

In general, a “promoter” or “promoter sequence” is a DNA regulatory region capable of binding an RNA polymerase in a cell (e.g., directly or through other promoter-bound proteins or substances) and initiating transcription of a coding sequence. A promoter may be operably linked to other expression control sequences, including enhancer and repressor sequences and/or with a polynucleotide of the disclosure. Examples of promoters which may be used to control gene expression include, but are not limited to, cytomegalovirus (CMV) promoter (U.S. Pat. Nos. 5,385,839 and 5,168,062), the SV40 early promoter region (Benoist, et al., (1981) Nature 290:304-310), the promoter contained in the 3′ long terminal repeat of Rous sarcoma virus (Yamamoto, et al., (1980) Cell 22:787-797), the herpes thymidine kinase promoter (Wagner, et al., (1981) Proc. Natl. Acad. Sci. USA 78:1441-1445), the regulatory sequences of the metallothionein gene (Brinster, et al., (1982) Nature 296:39-42); prokaryotic expression vectors such as the beta-lactamase promoter (Vllla-Komaroff, et al., (1978) Proc. Natl. Acad. Sci. USA 75:3727-3731), or the tac promoter (DeBoer, et al., (1983) Proc. Natl. Acad. Sci. USA 80:21-25); see also “Useful proteins from recombinant bacteria” in Scientific American (1980) 242:74-94; and promoter elements from yeast or other fungi such as the Ga/4 promoter, the ADC (alcohol dehydrogenase) promoter, PGK (phosphoglycerol kinase) promoter or the alkaline phosphatase promoter.

A polynucleotide encoding a polypeptide is “operably linked” to a promoter or other expression control sequence when, in a cell or other expression system, the sequence directs RNA polymerase mediated transcription of the coding sequence into RNA, preferably mRNA, which then may be RNA spliced (if it contains introns) and, optionally, translated into a protein encoded by the coding sequence.

The present disclosure includes polynucleotides encoding immunoglobulin polypeptide chains which are variants of those whose nucleotide sequence is specifically set forth herein. A “variant” of a polynucleotide refers to a polynucleotide comprising a nucleotide sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical to a referenced nucleotide sequence that is set forth herein; when the comparison is performed by a BLAST algorithm wherein the parameters of the algorithm are selected to give the largest match between the respective sequences over the entire length of the respective reference sequences (e.g., expect threshold: 10; word size: 28; max matches in a query range: 0; match/mismatch scores: 1, -2; gap costs: linear). In an embodiment of the disclosure, a variant of a nucleotide sequence specifically set forth herein comprises one or more (e.g., 1, 2, 3, 4, 5, 6, 7, 8, 9, 10, 11 or 12) point mutations, insertions (e.g., in frame insertions) or deletions (e.g., in frame deletions) of one or more nucleotides. Such mutations may, in an embodiment of the disclosure, be missense or nonsense mutations. In an embodiment of the disclosure, such a variant polynucleotide encodes an immunoglobulin polypeptide chain which can be incorporated into an PD-L1 Binding Arm and/or CD28 Binding Arm, i.e., such that the protein retains specific binding to PD-L1 and/or CD28.

Eukaryotic and prokaryotic host cells, including mammalian cells, may be used as hosts for expression of an PD-L1×CD28 antigen-binding protein (e.g., antibody or antigen-binding fragment thereof) or an antigen-binding arm thereof. Such host cells are well known in the art and many are available from the American Type Culture Collection (ATCC). These host cells include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Other cell lines that may be used include insect cell lines (e.g., Spodoptera frugiperda or Trichoplusia ni), amphibian cells, bacterial cells, plant cells and fungal cells. Fungal cells include yeast and filamentous fungus cells including, for example, Pichia, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. The present disclosure includes an isolated host cell (e.g., a CHO cell or any type of host cell set forth above) comprising an an anti-PD-L1×anti-CD28 antigen-binding protein of the present disclosure, such as REGN6192, REGN6193 and REGN6194 and the anti-PD-L1×anti-CD28 antigen-binding proteins shown in Table 9, or one or more polynucleotide molecules encoding an immunoglobulin (Ig) heavy and/or light chain thereof); and/or one or more polynucleotides encoding the PD-L1 binding arm and CD28 binding arm of a multispecific antigen-binding protein of the present disclosure.

The present disclosure also includes a cell which is expressing an PD-L1 and/or CD28 or an antigenic fragment or fusion thereof (e.g., Hiss, Fc and/or myc) which is bound by an PD-L1×CD28 antigen-binding protein of the present disclosure (e.g., an antibody or antigen-binding fragment thereof), for example, REGN6194, as well as any of bispecific antibodies disclosed herein.

There are several methods by which to produce recombinant antibodies which are known in the art. One example of a method for recombinant production of antibodies is disclosed in U.S. Pat. No. 4,816,567. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art. See, for example, U.S. Pat. Nos. 4,399,216; 4,912,040; 4,740,461 and 4,959,455.

The present disclosure includes recombinant methods for making an anti-PD-L1×anti-CD28 (e.g., REGN6194) antigen-binding protein of the present disclosure, such as an antibody or antigen-binding fragment thereof of the present disclosure, or an immunoglobulin chain thereof, comprising
(i) introducing, into a host cell, one or more polynucleotides encoding the light and heavy immunoglobulin chains encoding the PD-L1×CD28 antigen-binding protein's antigen-binding arms for example, wherein the polynucleotide is in a vector; and/or integrates into the host cell chromosome and/or is operably linked to a promoter;
(ii) culturing the host cell (e.g., CHO or Pichia or Pichia pastoris) under conditions favorable to expression of the polynucleotide and,
(iii) optionally, isolating the antigen-binding protein (e.g., antibody or antigen-binding fragment) or chain from the host cell and/or medium in which the host cell is grown. The present disclosure also includes PD-L1×CD28 antigen-binding proteins, such as antibodies and antigen-binding fragments thereof, which are the product of the production methods set forth herein, and, optionally, the purification methods set forth herein.

In an embodiment of the disclosure, a method for making an PD-L1×CD28 (e.g., REGN6194) antigen-binding protein, e.g., antibody or antigen-binding fragment thereof, includes a method of purifying the antigen-binding protein, e.g., by column chromatography, precipitation and/or filtration. As discussed, the product of such a method also forms part of the present disclosure.

### Sequence Variants

The antibodies and bispecific antigen-binding molecules of the present disclosure may comprise one or more amino acid substitutions, insertions and/or deletions in the framework and/or CDR regions of the heavy and light chain variable domains as compared to the corresponding germline sequences from which the individual antigen-binding domains were derived. Such mutations can be readily ascertained by comparing the amino acid sequences disclosed herein to germ line sequences available from, for example, public antibody sequence databases. The antigen-binding molecules of the present disclosure may comprise antigen-binding fragments which are derived from any of the exemplary amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antigen-binding domain was originally derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germ line sequence from which the antigen-binding domain was originally derived). Furthermore, the antigen-binding domains may contain any combination of two or more germline mutations within the framework and/or CDR regions, e.g., wherein certain individual residues are mutated to the corresponding residue of a particular germ line sequence while certain other residues that differ from the original germ line sequence are maintained or are mutated to the corresponding residue of a different germline sequence. Once obtained, antigen-binding domains that contain one or more germline mutations can be easily tested for one or more desired properties such as improved binding specificity, increased binding affinity, improved or enhanced antagonistic or agonistic biological properties (as the case may be), reduced immunogenicity, etc. Bispecific antigen-binding molecules comprising one or more antigen-binding domains obtained in this general manner are encompassed within the present disclosure.

The present disclosure also includes antigen-binding molecules wherein one or both antigen-binding domains comprise variants of any of the HCVR, LCVR, and/or CDR amino acid sequences disclosed herein having one or more conservative substitutions. For example, the present disclosure includes antigen-binding molecules comprising an antigen-binding domain having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445. A “moderately conservative” replacement is any change having a nonnegative value in the PAM250 log-likelihood matrix.

The present disclosure also includes antigen-binding molecules comprising an antigen-binding domain with an HCVR, LCVR, and/or CDR amino acid sequence that is substantially identical to any of the HCVR, LCVR, and/or CDR amino acid sequences disclosed herein. The term “substantial identity” or “substantially identical,” when referring to an amino acid sequence means that two amino acid sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) Methods Mol. Biol. 132: 185-219). Another preferred algorithm when comparing a sequence of the disclosure to a database containing a large number of sequences from different organisms is the computer program BLAST, especially BLASTP or TBLASTN, using default parameters. See, e.g., Altschul et al. (1990) J. Mol. Biol. 215:403-410; Altschul et al. (1997) Nucleic Acids Res. 25:3389-402.

### Antibodies Comprising Fc Variants

According to certain embodiments of the present disclosure, anti-PD-L1×anti-CD28 bispecific antigen-binding molecules are provided comprising an Fc domain comprising one or more mutations which enhance or diminish antibody binding to the FcRn receptor, e.g., at acidic pH as compared to neutral pH. For example, the present disclosure includes antibodies and antigen-binding molecules comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position
250 (e.g., E or Q);
250 and 428 (e.g., L or F);
252 (e.g., L/Y/F/W or T),
254 (e.g., S or T), and/or
256(e.g., S/R/Q/E/D or T);
or a modification at position
428 and/or 433 (e.g., H/L/R/S/P/Q or K) and/or
434 (e.g., H/F or Y);
or a modification at position
250 and/or 428;
or a modification at position
307 or 308 (e.g., 308F, V308F), and/or
434.

In one embodiment, the modification comprises a
428L (e.g., M428L) and 434S (e.g., N434S) modification;
a 428L, 2591 (e.g., V2591), and 308F (e.g., V308F) modification;
a 433K (e.g., H433K) and a 434 (e.g., 434Y) modification;
a 252, 254, and 256 (e.g., 252Y, 254T, and 256E) modification;
a 2500 and 428L modification (e.g., T250Q and M428L); and/or
a 307 and/or 308 modification (e.g., 308F or 308P).

For example, the present disclosure includes PD-L1×CD28 bispecific antigen-binding molecules comprising an Fc domain comprising one or more pairs or groups of mutations selected from the group consisting of:
2500 and 248L (e.g., T250Q and M248L);
252Y, 254T and 256E (e.g., M252Y, S254T and T256E);
428L and 434S (e.g., M428L and N434S); and
433K and 434F (e.g., H433K and N434F).

The present disclosure also includes bispecific antigen-binding molecules comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). See, for example, U.S. Pat. No. 8,586,713. Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies.

All possible combinations of the foregoing Fc domain mutations, and other mutations within the antibody variable domains disclosed herein, are contemplated within the scope of the present disclosure.

### Biological Characteristics of the Bispecific Antibodies and Antigen-Binding Molecules

The present disclosure includes antibodies and antigen-binding fragments thereof that bind human CD28 and PD-L1 with high affinity. The present disclosure also includes antibodies and antigen-binding fragments thereof that bind human CD28 and/or PD-L1 with medium or low affinity, depending on the therapeutic context and particular targeting properties that are desired. For example, in the context of a bispecific antigen-binding molecule, wherein one arm binds CD28 and another arm binds a target antigen (e.g., PD-L1), it may be desirable for the target antigen-binding arm to bind the target antigen with high affinity while the anti-CD28 arm binds CD28 with only moderate or low affinity. In this manner, preferential targeting of the antigen-binding molecule to cells expressing the target antigen may be achieved while avoiding general/untargeted CD28 binding and the consequent adverse side effects associated therewith.

According to certain embodiments, the present disclosure includes antibodies and antigen-binding fragments of antibodies that bind human CD28 (e.g., at 25° C.) with a K_{D} of less than about 200 nM as measured by surface plasmon resonance, e.g., using an assay format as defined in Example 2 herein. In certain embodiments, the antibodies or antigen-binding fragments of the present disclosure bind CD28 with a KD of less than about 100 nM, less than about 90 nM, less than about 80 nM, less than about 60 nM, less than about 40 nM, less than about 30 nM, less than 20 nM, less than 10 nM, or less than 5 nM as measured by surface plasmon resonance, e.g., using an assay format as defined in Example 2 herein, or a substantially similar assay. In certain embodiments, the antibodies or antigen-binding fragments of the present disclosure bind CD28 with a KD of about 5 nM to about 50 nM.

The present disclosure also includes antibodies and antigen-binding fragments thereof that bind CD28 with a dissociative half-life (t½) of greater than about 3 minutes as measured by surface plasmon resonance at 25° C. or 37° C., e.g., using an assay format as defined in the Examples herein, or a substantially similar assay. In certain embodiments, the antibodies or antigen-binding fragments of the present disclosure bind CD28 with a t½ of greater than about 5 minutes, greater than about 10 minutes, greater than about 20 minutes, greater than about 30 minutes, greater than about 40 minutes, or greater than about 50 minutes, as measured by surface plasmon resonance at 25° C. or 37° C., e.g., using an assay format as defined in Example 2 herein, or a substantially similar assay.

According to certain embodiments, the present disclosure includes antibodies and antigen-binding fragments of antibodies that bind human PD-L1 (e.g., at 25° C.) with a K_{D} of less than about 1 nM as measured by surface plasmon resonance, e.g., using an assay format as defined in Example 2 herein. In certain embodiments, the antibodies or antigen-binding fragments of the present disclosure bind PD-L1 with a KD of less than about 1 nM, less than about 0.9 nM, less than about 0.8 nM, less than about 0.6 nM, less than about 0.4 nM, less than about 0.3 nM, less than 0.2 nM, less than 0.1 nM, or less than 0.05 nM as measured by surface plasmon resonance, e.g., using an assay format as defined in Example 2 herein, or a substantially similar assay. In certain embodiments, the antibodies or antigen-binding fragments of the present disclosure bind PD-L1 with a KD of about 0.05 nM to about 0.2 nM.

The present disclosure also includes antibodies and antigen-binding fragments thereof that bind PD-L1 with a dissociative half-life (t½) of greater than about 30 minutes as measured by surface plasmon resonance at 25° C. or 37° C., e.g., using an assay format as defined in the Examples herein, or a substantially similar assay. In certain embodiments, the antibodies or antigen-binding fragments of the present disclosure bind PD-L1 with a t½ of greater than about 30 minutes, greater than about 60 minutes, greater than about 120 minutes, greater than about 150 minutes, greater than about 180 minutes, or greater than about 200 minutes, as measured by surface plasmon resonance at 25° C. or 37° C., e.g., using an assay format as defined in Example 2 herein, or a substantially similar assay

The present disclosure includes bispecific antigen-binding molecules (e.g., bispecific antibodies) which are capable of simultaneously binding to human CD28 and human PD-L1. According to certain embodiments, the bispecific antigen-binding molecules of the disclosure specifically interact with cells that express CD28 and/or PD-L1. The extent to which a bispecific antigen-binding molecule binds cells that express CD28 and/or PD-L1 can be assessed by fluorescence activated cell sorting (FACS), as illustrated in Example 3 herein. For example, the present disclosure includes bispecific antigen-binding molecules which specifically bind human cell lines which express CD28 but not PD-L1 (e.g., Jurkat cell), and HEK cell lines which do not express PD-L1 and CD28. In some embodiments, the bispecific antigen-binding molecules bind to CD28-expressing human or cynomolgus T-cells with an EC50 value less than 1×10⁻⁵ M. In some embodiments, the bispecific antigen-binding molecules bind to CD28-expressing human or cynomolgus T-cells with an EC50 value of 1×10⁻¹² M to 1×10⁻⁵ M. In certain embodiments, the bispecific antigen-binding molecules bind to CD28-expressing human or cynomolgus T-cells with an EC50 value of 1×10⁻¹² M to 1×10⁻⁹ M. In certain embodiments, the bispecific antigen-binding molecules bind to the surface of cell lines expressing PD-L1 with an EC₅₀ of less than about 2.5×10⁻⁸ M. The binding of the bispecific antigen-binding molecules to the surface of cells or cell lines can be measured by an in vitro FACS binding assay as described in the Examples.

The present disclosure includes PD-L1×CD28 bispecific antigen-binding molecules which are capable of depleting tumor cells in a subject. For example, according to certain embodiments, PD-L1×CD28 bispecific antigen-binding molecules are provided, wherein a single administration of the antigen-binding molecule to a subject at a therapeutically effective dose causes a reduction in the number of tumor cells in the subject.

The present disclosure includes anti-PD-L1×anti-CD28 bispecific antigen-binding molecules which are capable of binding to PD-L1 expressed on cells surface. A variety of tumor cells express PD-L1, including breast tumor cells (e.g., HeLa, MCF-7 and MDA-MB-231), melanoma cells (e.g., A375), lung tumor cells (e.g., HCC44), ovarian cancer cells (e.g., ES-2, SNU-8, MCAS), pancreatic cancer cells (eig., SNU-324), prostate cancer cells (e.g., DU145). As such, the bispecific antibodies of the disclosure may prove useful in treating a multitude of cancer indications.

The present disclosure includes anti-PD-L1×anti-CD28 bispecific antigen-binding molecules which are capable of activating T cells by engaging PD-L1 on target cells and CD28 on T-cells (see Example 3). For example, binding of the anti-PD-L1×anti-CD28 bispecific antigen-binding molecules to T cells can lead to an increase in IL-2 release (see Example 4). As such, the bispecific antigen-binding molecules of the disclosure may prove useful in promoting a T-cell mediated immune response.

The present disclosure includes anti-PD-L1×anti-CD28 bispecific antigen-binding molecules which are capable of blocking interaction of PD-L1 with PD-1 (see Example 5). As such, the bispecific antigen-binding molecules of the disclosure can be useful for inhibiting the immune checkpoint pathway and reducing T-cell exhaustion, thereby promoting a T-cell mediated immune response.

The present disclosure includes anti-PD-L1×anti-CD28 bispecific antigen-binding molecules which are capable of enhancing the cytotoxic potency of anti-tumor associated antigen (TAA)×anti-CD3 bispecific antibodies across a variety of tumor cells (See Example 4). In certain embodiments, the TAA is selected from the group consisting of AFP, ALK, BAGE proteins, BCMA, BIRC5 (survivin), BIRC7, β-catenin, brc-abl, BRCA1, BORIS, CA9, carbonic anhydrase IX, caspase-8, CALR, CCR5, CD19, CD20 (MS4A1), CD22, CD30, CD40, CDK4, CEA, CTLA4, cyclin-B1, CYP1 B1, EGFR, EGFRvIII, ErbB2/Her2, ErbB3, ErbB4, ETV6-AML, EpCAM, EphA2, Fra-1, FOLR1, GAGE proteins (e.g., GAGE-1, -2), GD2, GD3, GloboH, glypican-3, GM3, gp100, Her2, HLA/B-raf, HLA/k-ras, HLA/MAGE-A3, hTERT, LMP2, MAGE proteins (e.g., MAGE-1, -2, -3, -4, -6, and -12), MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, Ras, RGS5, Rho, SART-1, SART-3, STEAP1, STEAP2, TAG-72, TGF-β, TMPRSS2, Thompson-nouvelle antigen (Tn), TRP-1, TRP-2, tyrosinase, and uroplakin-3. The anti-PD-L1×anti-CD28 antibodies may also prove useful when combined with a checkpoint inhibitor, for example, an antibody to PD-1, or any other checkpoint inhibitor.

### Epitope Mapping and Related Technologies

The epitope on CD28 or PD-L1 to which the antigen-binding molecules of the present disclosure bind may consist of a single contiguous sequence of 3 or more (e.g., 3, 4, 5, 6, 7, 8, 9, 10, 11, 12, 13, 14, 15, 16, 17, 18, 19, 20 or more) amino acids of a CD28 protein or a PD-L1 protein. Alternatively, the epitope may consist of a plurality of non-contiguous amino acids (or amino acid sequences) of CD28 or PD-L1. The antibodies of the disclosure may interact with amino acids contained within a CD28 monomer, or may interact with amino acids on two different CD28 chains of a CD28 dimer. The term “epitope,” as used herein, refers to an antigenic determinant that interacts with a specific antigen-binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

Various techniques known to persons of ordinary skill in the art can be used to determine whether an antigen-binding domain of an antibody “interacts with one or more amino acids” within a polypeptide or protein. Exemplary techniques that can be used to determine an epitope or binding domain of a particular antibody or antigen-binding domain include, e.g., routine crossblocking assay such as that described in Antibodies, Harlow and Lane (Cold Spring Harbor Press, Cold Spring Harb., NY), point mutagenesis (e.g., alanine scanning mutagenesis, arginine scanning mutagenesis, etc.), peptide blots analysis (Reineke, 2004, Methods Mol Biol 248:443-463), protease protection, and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267(2):252-259; Engen and Smith (2001) Anal. Chem. 73:256A-265A. Alternatively, in certain embodiments, the protein of interest binds to the antibody, followed by hydrogen-deuterium exchange. After dissociation of the antibody, the target protein is subjected to protease cleavage and mass spectrometry analysis, thereby revealing the non-deuterium-labeled residues which correspond to the specific amino acids with which the antibody interacts. X-ray crystal structure analysis can also be used to identify the amino acids within a polypeptide with which an antibody interacts.

The present disclosure further includes anti-CD28 and anti-PD-L1 antibodies that bind to the same epitope as any of the specific exemplary antibodies described herein (e.g., antibodies comprising any of the amino acid sequences as set forth in Tables 1, 3, 6, and 9). Likewise, the present disclosure also includes anti-CD28 and/or anti-PD-L1 antibodies that compete for binding to CD28 and/or PD-L1 with any of the specific exemplary antibodies described herein (e.g., antibodies comprising any of the amino acid sequences as set forth in Tables 1, 3, 6, and 9 herein).

The present disclosure also includes bispecific antigen-binding molecules comprising a first antigen-binding domain that specifically binds human CD28, and a second antigen-binding fragment that specifically binds human PD-L1, wherein the first antigen-binding domain binds to the same epitope on CD28 as any of the specific exemplary CD28-specific antigen-binding domains described herein, and/or wherein the second antigen-binding domain binds to the same epitope on PD-L1 as any of the specific exemplary PD-L1-specific antigen-binding domains described herein. Likewise, the present disclosure also includes bispecific antigen-binding molecules comprising a first antigen-binding domain that specifically binds human CD28, and a second antigen-binding fragment that specifically binds human PD-L1, wherein the first antigen-binding domain competes for binding to CD28 with any of the specific exemplary CD28-specific antigen-binding domains described herein, and/or wherein the second antigen-binding domain competes for binding to PD-L1 with any of the specific exemplary PD-L1-specific antigen-binding domains described herein.

One can easily determine whether a particular antigen-binding molecule (e.g., antibody) or antigen-binding domain thereof binds to the same epitope as, or competes for binding with, a reference antigen-binding molecule of the present disclosure by using routine methods known in the art. For example, to determine if a test antibody binds to the same epitope on CD28 (or PD-L1) as a reference bispecific antigen-binding molecule of the present disclosure, the reference bispecific molecule is first allowed to bind to a CD28 protein (or PD-L1 protein). Next, the ability of a test antibody to bind to the CD28 (or PD-L1) molecule is assessed. If the test antibody is able to bind to CD28 (or PD-L1) following saturation binding with the reference bispecific antigen-binding molecule, it can be concluded that the test antibody does not compete for binding to CD28 (or PD-L1) with the reference bispecific antigen-binding molecule and/or that there is steric interference between antibodies that are binding different sites on the antigen. On the other hand, if the test antibody is not able to bind to the CD28 (or PD-L1) molecule following saturation binding with the reference bispecific antigen-binding molecule, then the test antibody competes for binding to CD28 (or PD-L1) with the reference bispecific antigen-binding molecule of the disclosure. Additional routine experimentation (e.g., peptide mutation and binding analyses) can then be carried out to confirm whether the observed lack of binding of the test antibody is in fact due to binding to the same epitope as the reference bispecific antigen-binding molecule or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present disclosure, two antigen-binding proteins compete for binding to an antigen if, e.g., a 1-, 5-, 10-, 20- or 100-fold excess of one antigen-binding protein inhibits binding of the other by at least 50% but preferably 75%, 90% or even 99% as measured in a competitive binding assay (see, e.g., Junghans et al., Cancer Res. 1990:50:1495-1502). Alternatively, two antigen-binding proteins may bind to the same epitope if essentially all amino acid mutations in the antigen that reduce or eliminate binding of one antigen-binding protein reduce or eliminate binding of the other. Two antigen-binding proteins may have “overlapping epitopes” if only a subset of the amino acid mutations that reduce or eliminate binding of one antigen-binding protein reduce or eliminate binding of the other.

To determine if an antibody or antigen-binding domain thereof competes for binding with a reference antigen-binding molecule, the above-described binding methodology is performed in two orientations: In a first orientation, the reference antigen-binding molecule is allowed to bind to a CD28 protein (or PD-L1 protein) under saturating conditions followed by assessment of binding of the test antibody to the CD28 (or PD-L1) molecule. In a second orientation, the test antibody is allowed to bind to a CD28 (or PD-L1) molecule under saturating conditions followed by assessment of binding of the reference antigen-binding molecule to the CD28 (or PD-L1) molecule. If, in both orientations, only the first (saturating) antigen-binding molecule is capable of binding to the CD28 (or PD-L1) molecule, then it is concluded that the test antibody and the reference antigen-binding molecule compete for binding to CD28 (or PD-L1). As will be appreciated by a person of ordinary skill in the art, an antibody that competes for binding with a reference antigen-binding molecule may not necessarily bind to the same epitope as the reference antibody, but may sterically block binding of the reference antibody by binding an overlapping or adjacent epitope.

### Preparation of Antigen-Binding Domains and Construction of Bispecific Molecules

Antigen-binding domains specific for particular antigens can be prepared by any antibody generating technology known in the art. Once obtained, two different antigen-binding domains, specific for two different antigens (e.g., CD28 and PD-L1), can be appropriately arranged relative to one another to produce a bispecific antigen-binding molecule of the present disclosure using routine methods. (A discussion of exemplary bispecific antibody formats that can be used to construct the bispecific antigen-binding molecules of the present disclosure is provided elsewhere herein). In certain embodiments, one or more of the individual components (e.g., heavy and light chains) of the multispecific antigen-binding molecules of the disclosure are derived from chimeric, humanized or fully human antibodies. Methods for making such antibodies are well known in the art. For example, one or more of the heavy and/or light chains of the bispecific antigen-binding molecules of the present disclosure can be prepared using VELOCIMMUNE™ technology. Using VELOCIMMUNE™ technology (or any other human antibody generating technology), high affinity chimeric antibodies to a particular antigen (e.g., CD28 or PD-L1) are initially isolated having a human variable region and a mouse constant region. The antibodies are characterized and selected for desirable characteristics, including affinity, selectivity, epitope, etc. The mouse constant regions are replaced with a desired human constant region to generate fully human heavy and/or light chains that can be incorporated into the bispecific antigen-binding molecules of the present disclosure.

Genetically engineered animals may be used to make human bispecific antigen-binding molecules. For example, a genetically modified mouse can be used which is incapable of rearranging and expressing an endogenous mouse immunoglobulin light chain variable sequence, wherein the mouse expresses only one or two human light chain variable domains encoded by human immunoglobulin sequences operably linked to the mouse kappa constant gene at the endogenous mouse kappa locus. Such genetically modified mice can be used to produce fully human bispecific antigen-binding molecules comprising two different heavy chains that associate with an identical light chain that comprises a variable domain derived from one of two different human light chain variable region gene segments. (See, e.g., US 2011/0195454 for a detailed discussion of such engineered mice and the use thereof to produce bispecific antigen-binding molecules).

### Bioequivalents

The present disclosure encompasses antigen-binding molecules having amino acid sequences that vary from those of the described antibodies but that retain the ability to bind CD28 and PD-L1. Such variant molecules comprise one or more additions, deletions, or substitutions of amino acids when compared to parent sequence, but exhibit biological activity that is essentially equivalent to that of the described antigen-binding molecules. Likewise, the antigen-binding molecules-encoding DNA sequences of the present disclosure encompass sequences that comprise one or more additions, deletions, or substitutions of nucleotides when compared to the disclosed sequence, but that encode an antigen-binding molecule that is essentially bioequivalent to the described antigen-binding molecules of the disclosure. Examples of such variant amino acid and DNA sequences are discussed above.

The present disclosure includes antigen-binding molecules that are bioequivalent to any of the exemplary antigen-binding molecules set forth herein. Two antigen-binding proteins or antibodies are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the exemplary bispecific antigen-binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include the exemplary bispecific antigen-binding molecules set forth herein comprising amino acid changes which modify the glycosylation characteristics of the antibodies, e.g., mutations which eliminate or remove glycosylation.

### Species Selectivity and Species Cross-Reactivity

The present disclosure, according to certain embodiments, provides antigen-binding molecules that bind to human CD28 but not to CD28 from other species. The present disclosure also provides antigen-binding molecules that bind to human PD-L1 but not to PD-L1 from other species. The present disclosure also includes antigen-binding molecules that bind to human CD28 and to CD28 from one or more non-human species; and/or antigen-binding molecules that bind to human PD-L1 and to PD-L1 from one or more non-human species.

According to certain exemplary embodiments, antigen-binding molecules are provided which bind to human CD28 and human PD-L1 and may bind or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee CD28 and or PD-L1. For example, in an exemplary embodiment, bispecific antigen-binding molecules are provided comprising a first antigen-binding domain that binds human CD28 and cynomolgus CD28, and a second antigen-binding domain that specifically binds human PD-L1.

### Immunoconjugates

The disclosure encompasses PD-L1×CD28 antigen-binding proteins, e.g., antibodies or antigen-binding fragments, such as REGN6194, conjugated to another moiety, e.g., a therapeutic moiety (an “immunoconjugate”). In an embodiment, an PD-L1×CD28 antigen-binding protein, e.g., antibody or antigen-binding fragment, is conjugated to any of the further therapeutic agents set forth herein. As used herein, the term “immunoconjugate” refers to an antigen-binding protein, e.g., an antibody or antigen-binding fragment, which is chemically or biologically linked to another antigen-binding protein, a drug, a radioactive agent, a reporter moiety, an enzyme, a peptide, a protein or a therapeutic agent.

In certain embodiments, the therapeutic moiety may be a cytotoxin, a chemotherapeutic drug, an immunosuppressant or a radioisotope. Cytotoxic agents include any agent that is detrimental to cells. Examples of suitable cytotoxic agents and chemotherapeutic agents for forming immunoconjugates are known in the art, (see for example, WO 05/103081).

### Therapeutic Uses of the Antigen-Binding Molecules

The bispecific antibodies and antigen-binding molecules of the disclosure (and therapeutic compositions comprising the same) are useful, inter alia, for treating any disease or disorder in which stimulation, activation and/or targeting of an immune response would be beneficial. In particular, the PD-L1×CD28 bispecific antigen-binding molecules of the present disclosure may be used for the treatment, prevention and/or amelioration of a hyperproliferative disease, for example, cancer. in certain embodiments, the present disclosure provides methods for treating cancer in a subject, comprising administering a therapeutically effective dose of PD-L1×CD28 antigen-binding protein, e.g., REGN6194,to the subject

A hyperproliferative disease, for the purposes herein, refers to a disease characterized by abnormal, excessive and/or uncontrolled cell growth, e.g., a cancer wherein the cells express PD-L1. For example, hyperproliferative diseases include cancers. Exemplary cancers include, but are not limited to esophageal carcinoma, lung squamous cell carcinoma, lung adenocarcinoma, cervical squamous cell carcinoma, glioma, thyroid cancer, lung cancer (e.g., non-small cell lung cancer), colorectal cancer, colon cancer, bladder cancer, rectal cancer, head and neck cancer, stomach cancer, liver cancer, pancreatic cancer, renal cancer, urothelial cancer, prostate cancer, testis cancer, breast cancer, cervical cancer, endometrial cancer, ovarian cancer, gastroesophageal cancer, (e.g., gastroesophageal adenocarcinoma), basal cell carcinoma, diffuse large B cell lymphoma, or multiple myeloma and melanoma. Accordingly, the antibodies and the bispecific antigen-binding molecules of the present disclosure can be used in treating a wide range of cancers.

Cancer characterized by solid tumor cells or cancerous blood cells may be an PD-L1-expressing cancer e.g., wherein PD-L1 expression in the cells of the particular subject to be treated has been confirmed, includes esophageal carcinoma, lung squamous cell carcinoma, lung adenocarcinoma, cervical squamous cell carcinoma, endometrial adenocarcinoma, bladder urothelial carcinoma, lung cancer (e.g., non-small cell lung cancer), colorectal cancer, rectal cancer, endometrial cancer, skin cancer (e.g., head & neck squamous cell carcinoma), brain cancer (e.g., glioblastoma multiforme), breast cancer, gastroesophageal cancer, (e.g., gastroesophageal adenocarcinoma), prostate cancer, ovarian cancer, melanoma, basal cell carcinoma, cervical cancer, diffuse large B cell lymphoma, and/or multiple myeloma.

The antigen-binding molecules of the present disclosure may also be used to treat, e.g., primary and/or metastatic tumors arising in the colon, lung, breast, ovary, kidney, and bladder (or from any cancer discussed herein).

The antigen-binding proteins of the present disclosure may also be used to treat residual cancer in a subject. As used herein, the term “residual cancer” means the existence or persistence of one or more cancerous cells in a subject following treatment with an anti-cancer therapy.

As used herein, the term “subject” refers to a mammal (e.g., rat, mouse, cat, dog, cow, sheep, horse, goat, rabbit), preferably a human, for example, in need of prevention and/or treatment of cancer. The subject may have a cancer, be predisposed to developing such a condition, and/or would benefit from administration of a bispecific antibody or antigen-binding fragment thereof of the present disclosure. In one embodiment, the subject may have, or be at risk of developing, a hyperproliferative disease.

Methods for treating or preventing a cancer (e.g., a PD-L1-expressing cancer) in a subject in need of said treatment or prevention by administering a therapeutically effective dose amount PD-L1×CD28 antigen-binding protein, in association with an additional therapeutic agent are part of the present disclosure. Additional therapeutic agents are disclosed elsewhere herein.

An “effective” or “therapeutically effective” amount of PD-L1×CD28 antigen-binding protein, e.g., antibody or antigen-binding fragment, for treating or preventing a hyperproliferative disease, such as cancer, is the amount of the antigen-binding protein sufficient to alleviate one or more signs and/or symptoms of the disease in the treated subject, whether by inducing the regression or elimination of such signs and/or symptoms or by inhibiting the progression of such signs and/or symptoms. In an embodiment of the disclosure, a therapeutically effective dose of PD-L1×CD28 antigen-binding protein is 0.1-2000 mg. The dose amount may vary depending upon the age and the size of a subject to be administered, target disease, conditions, route of administration, and the like. In certain embodiments, the initial dose may be followed by administration of a second or a plurality of subsequent doses of antigen-binding protein in an amount that can be approximately the same or less or more than that of the initial dose, wherein the subsequent doses may be separated by 1-8 weeks.

The dose of antigen-binding molecule administered to a patient may vary depending upon the age and the size of the patient, target disease, conditions, route of administration, and the like. The preferred dose is typically calculated according to body weight or body surface area. Depending on the severity of the condition, the frequency and the duration of the treatment can be adjusted. Effective dosages and schedules for administering a bispecific antigen-binding molecule may be determined empirically; for example, patient progress can be monitored by periodic assessment, and the dose adjusted accordingly.

### Combination Therapies

The bispecific antigen-binding molecules of the present disclosure may be used in combination with one or more agents, for example, in treating a cancer in a subject. In certain embodiments, the bispecific antigen-binding molecules may be administered in combination with one or more agents, for example, a corticosteroid, to reduce or ameliorate one or more adverse side effects, e.g., cytokine storm. In certain embodiments, the bispecific antigen-binding molecules may be administered in combination with one or more therapeutic agents or therapies for enhanced efficacy in treating cancer. Exemplary additional therapeutic agents or therapies that may be combined with or administered in combination with an antigen-binding molecule of the present disclosure include, e.g., chemotherapy (e.g., anti-cancer chemotherapy, for example, paclitaxel, docetaxel, vincristine, cisplatin, carboplatin or oxaliplatin), radiation therapy, surgery, a checkpoint inhibitor, a PD-1 inhibitor(e.g., an anti-PD-1 antibody such as pembrolizumab, nivolumab, or cemiplimab), a CTLA-4 inhibitor, LAG3 inhibitor, TIM3 inhibitor, a GITR agonist, OX40 agonist, 4-1 BB agonist, an oncolytic virus, a cancer vaccine, a CAR-T cell, a nucleic acid therapeutic, a stem cell transplant, a modified IL2, modified IL12, IL15, IL6 inhibitor (e.g., sarilumab or tocilizumab), IL4R inhibitor (e.g., dupilumab), EGFR inhibitor, Ang2 inhibitor, VEGF inhibitor, a corticosteroid, a bispecific antibody or antigen-binding fragment thereof that binds CD3 and a tumor associated antigen (TAA) (e.g., MUC16 (mucin 16), PSMA, STEAP2, or any of the TAA disclosed herein). Exemplary bispecific antibodies comprising an antigen-binding domain that binds CD3 include, but are not limited to those described in, e.g., WO2017/053856A1, WO2014/047231A1, WO2018/067331A1 and WO2018/058001A1. PD-L1 is expressed in a wide range of cancers. Accordingly, the bispecific anti-PD-L1×CD28 antibodies of the present disclosure can be used in combination with a wide range of bispecific antibodies comprising an antigen-binding domain that binds CD3 in treatments of various cancers.

The additional agents may be administered just prior to, concurrent with, or shortly after the administration of an antigen-binding molecule of the present disclosure; (for purposes of the present disclosure, such an administration regimen is considered the administration of an antigen-binding molecule “in combination with” an additional agent or therapeutic agent or therapy).

### Pharmaceutical Formulations and Administration

The present disclosure provides compositions that include PD-L1×CD28 antigen-binding proteins and one or more ingredients; as well as methods of use thereof and methods of making such compositions. Pharmaceutical formulations (e.g., aqueous pharmaceutical formulations that include water) comprising an PD-L1×CD28 antigen-binding protein of the present disclosure and a pharmaceutically acceptable carrier or excipient are part of the present disclosure.

The pharmaceutical compositions of the disclosure can be formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

To prepare pharmaceutical formulations of the PD-L1×CD28 antigen-binding proteins, e.g., antibodies and antigen-binding fragments thereof (e.g., REGN6194), the antigen-binding protein is admixed with a pharmaceutically acceptable carrier or excipient. See, e.g., Remington's Pharmaceutical Sciences and U.S. Pharmacopeia: National Formulary, Mack Publishing Company, Easton, Pa. (1984); Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y. In an embodiment of the disclosure, the pharmaceutical formulation is sterile. Such compositions are part of the present disclosure.

Pharmaceutical formulations of the present disclosure include an PD-L1×CD28 antigen-binding protein and a pharmaceutically acceptable carrier including, for example, water, buffering agents, preservatives and/or detergents.

The scope of the present disclosure includes desiccated, e.g., freeze-dried compositions, comprising an PD-L1×CD28 antigen-binding protein, e.g., antibody or antigen-binding fragment thereof, or a pharmaceutical formulation thereof that includes a pharmaceutically acceptable carrier but substantially lacks water.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, rectal, intestinal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

As discussed herein, the present disclosure provides a vessel (e.g., a plastic or glass vial) or injection device (e.g., syringe, pre-filled syringe or autoinjector) comprising any of the PD-L1×CD28 antigen-binding proteins herein, e.g., antibodies or antigen-binding fragments thereof, or a pharmaceutical formulation comprising a pharmaceutically acceptable carrier or excipient thereof.

A pharmaceutical composition of the present disclosure can be delivered subcutaneously or intravenously with a standard needle and syringe. With respect to subcutaneous delivery, a pen delivery device, as known in the art, may be used in delivering a pharmaceutical composition of the present disclosure. Such a pen delivery device can be reusable or disposable.

Numerous reusable and disposable pens and autoinjector delivery devices have applications in the subcutaneous delivery of a pharmaceutical composition of the present disclosure. See e.g., AUTOPEN™ (Owen Mumford, Inc., Woodstock, UK) or the HUMIRA™ Pen (Abbott Labs, Abbott Park, IL)

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline and other isotonic solutions which may be used in combination with an appropriate solubilizing agent. Injectable oily mediums are also part of the present disclosure. Such oily mediums may be combined with a solubilizing agent.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 0.1 to about 2000 mg per dosage form in a unit dose; especially in the form of injection.

### Diagnostic Uses

The bispecific antibodies of the present disclosure may also be used to detect and/or measure CD28 or PD-L1, or CD28-expressing or PD-L1-expressing cells in a sample, e.g., for diagnostic purposes. For example, PD-L1×CD28 antibody or antigen-binding fragment thereof, may be used to diagnose a condition or disease characterized by aberrant expression (e.g., over-expression, under-expression, lack of expression, etc.) of CD28 or PD-L1. Exemplary diagnostic assays for CD28 or PD-L1 may comprise, e.g., contacting a sample, obtained from a patient, with an antibody of the disclosure, wherein the antibody is labeled with a detectable label or reporter molecule. Alternatively, an unlabeled antibody can be used in diagnostic applications in combination with a secondary antibody which is itself detectably labeled. The detectable label or reporter molecule can be a radioisotope, such as ³H, ¹⁴C ³²P, ³⁵S, or ¹²⁵I; a fluorescent or chemiluminescent moiety such as fluorescein isothiocyanate, or rhodamine; or an enzyme such as alkaline phosphatase, betagalactosidase, horseradish peroxidase, or luciferase. Specific exemplary assays that can be used to detect or measure CD28 or PD-L1 in a sample include enzyme-linked immunosorbent assay (ELISA), radioimmunoassay (RIA), and fluorescence-activated cell sorting (FACS). Samples that can be used in CD28 or PD-L1 diagnostic assays according to the present disclosure include any tissue or fluid sample obtainable from a patient which contains detectable quantities of CD28 or PD-L1 protein, or fragments thereof, under normal or pathological conditions. Generally, levels of CD28 or PD-L1 in a particular sample obtained from a healthy patient (e.g., a patient not afflicted with a disease or condition associated with abnormal CD28 or PD-L1 levels or activity) will be measured to initially establish a baseline, or standard, level of CD28 or PD-L1. This baseline level of CD28 or PD-L1 can then be compared against the levels of CD28 or PD-L1 measured in samples obtained from individuals suspected of having a CD28 or PD-L1 related disease or condition.

### EXAMPLES

The following examples are put forth so as to provide those of ordinary skill in the art with a complete disclosure and description of how to make and use the methods and compositions of the disclosure, and are not intended to limit the scope of what the inventors regard as their invention.

### Example 1: Construction of Anti-PD-L1×CD28 Antibodies

### Generation of Anti-PD-L1 Antibodies

Anti-PD-L1 antibodies were obtained by immunizing a genetically engineered mouse comprising DNA encoding human immunoglobulin heavy and kappa light chain variable regions with a human PD-L1 antigen.

Following immunization, splenocytes were harvested from each mouse and B-cell sorted (as described in US 2007/0280945) using a human PD-L1 fragment as the sorting agent that binds and identifies reactive antibodies (antigen-positive B-cells).

The antibodies were characterized and selected for desirable characteristics including affinity, selectivity, etc. The antibodies may have a desired constant region, for example, wild-type or modified hIgG1 or hIgG4 constant region. As will be appreciated by a person of skill in the art, an antibody with a particular constant region (e.g., modified hIgG1) may be converted to an antibody with a different constant region (e.g., modified hIgG4). While the constant region may vary according to specific use, high-affinity antigen-binding and target specificity characteristics reside in the variable region.

Table 1 sets forth the amino acid sequence identifiers of the heavy and light chain variable regions and CDRs of selected anti-PD-L1 antibodies of the disclosure. The corresponding nucleic acid sequence identifiers are set forth in Table 2.

TABLE 1
Amino Acid Sequence Identifiers for Selected Parental PD-L1 Monoclonal
Antibodies

| Antibody | SEQ ID NOS: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb9364 | 2 | 4 | 6 | 8 | 18 | 20 | 22 | 24 |
| mAb9373 | 42 | 44 | 46 | 48 | 58 | 60 | 62 | 64 |

TABLE 2
Nucleic Acid Sequence Identifiers for Selected Parental PD-L1 Monoclonal
Antibodies

| Antibody | SEQ ID NOS: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb9364 | 1 | 3 | 5 | 7 | 17 | 19 | 21 | 23 |
| mAb9373 | 41 | 43 | 45 | 47 | 57 | 59 | 61 | 63 |

### Generation of Anti-CD28 Antibodies

Anti-CD28 antibodies were obtained by immunizing a VELOCIMMUNE® mouse (i.e., an engineered mouse comprising DNA encoding human Immunoglobulin heavy and universal light chain variable regions) with human CD28 protein fused to the Fc portion of mouse IgG2a, or with cells expressing CD28 or with DNA encoding CD28.

The antibody immune response was monitored by a CD28-specific immunoassay. When a desired immune response was achieved, anti-CD28 antibodies were isolated directly from antigen-positive B cells, as described in U.S. Pat. No. 7,582,298.

The antibodies were characterized and selected for desirable characteristics including affinity, selectivity, etc. The antibodies may have a desired constant region, for example, wild-type or modified hIgG1 or hIgG4 constant region. As will be appreciated by a person of skill in the art, an antibody with a particular constant region (e.g., modified hIgG1) may be converted to an antibody with a different constant region (e.g., modified hIgG4). While the constant region may vary according to specific use, high-affinity antigen-binding and target specificity characteristics reside in the variable region.

Table 3 sets forth the amino acid sequence identifiers of the heavy and light chain variable regions and CDRs of selected anti-CD28 antibodies of the disclosure. The corresponding nucleic acid sequence identifiers are set forth in Table 4.

TABLE 3
Amino Acid Sequence Identifiers for Selected Parental CD28 Monoclonal
Antibodies

| Antibody | SEQ ID NOS: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb14193 | 10 | 12 | 14 | 16 | 18 | 20 | 22 | 24 |
| mAb14216 | 32 | 34 | 36 | 38 | 18 | 20 | 22 | 24 |
| mAb14226 | 50 | 52 | 54 | 56 | 58 | 60 | 62 | 64 |

TABLE 4
Nucleic Acid Sequence Identifiers for Selected Parental CD28 Antibodies

| Antibody | SEQ ID NOS: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb14193 | 9 | 11 | 13 | 15 | 17 | 19 | 21 | 23 |
| mAb14216 | 31 | 33 | 35 | 37 | 17 | 19 | 21 | 23 |
| mAb14226 | 49 | 51 | 53 | 55 | 57 | 59 | 61 | 63 |

Generation of Bispecific Antibodies (bsAbs) that Bind CD28 and PD-L1

Bispecific antibodies comprising an anti-PD-L1-specific binding domain and an anti-CD28-specific binding domain were constructed using standard methodologies, wherein the anti-PD-L1 antigen-binding domain and the anti-CD28 antigen-binding domain each comprise different, distinct HCVRs paired with a common LCVR. In some instances the bispecific antibodies were constructed utilizing a heavy chain from an anti-CD28 antibody, a heavy chain from an anti-PD-L1 antibody and a common light chain (either from the anti-PD-L1 antibody or from the anti-CD28 antibody). Table 5 summarizes the component parts (parental antibody designation) of selected bispecific PD-L1×CD28 antibodies. Tables 6 and 7 show the amino acid and nucleic acid identifiers, respectively of the selected bispecific antibodies. Table 8 shows the full-length heavy chain and light chain sequences of the selected bispecific antibodies. Additional bispecific antibodies that bind to PD-L1 and CD28 may be prepared using the parental monoclonal antibodies having the designations shown in Table 9.

TABLE 5
Summary of Component Parts of Selected Anti-PD-L1 x
Anti-CD28 Bispecific Antibodies

| Bispecific | Anti-PD-L1 | Anti-CD28 | Common |
|---|---|---|---|
| Antibody | Antigen- | Antigen- | Light Chain |
| Designation | Binding Domain | Binding Domain | Variable Region |
| REGN6192 | mAb9364 | mAb14193 | 1-39 |
| REGN6193 | mAb9364 | mAb14216 | 1-39 |
| REGN6194 | mAb9373 | mAb14226 | 3-20 |

TABLE 6
Amino Acid Sequence Identifiers of Selected Anti-PD-L1 × Anti-CD28 Bispecific
Antibodies

| | Anti-PD-L1 | | | | Anti-CD28 | | | | Common | | | |
|---|---|---|---|---|---|---|---|---|---|---|---|---|
| | Antigen-Binding | | | | Antigen-Binding | | | | Light Chain Variable | | | |
| Bispecific | Domain | | | | Domain | | | | Region | | | |
| Antibody | | HCDR | HCDR | HCDR | | HCDR | HCDR | HCDR | | 1 | 2 | 3 |
| Designation | HCVR | 1 | 2 | 3 | HCVR | 1 | 2 | 3 | LCVR | LCDR | LCDR | LCDR |
| REGN6192 | 2 | 4 | 6 | 8 | 10 | 12 | 14 | 16 | 18 | 20 | 22 | 24 |
| REGN6193 | 2 | 4 | 6 | 8 | 32 | 34 | 36 | 38 | 18 | 20 | 22 | 24 |
| REGN6194 | 42 | 44 | 46 | 48 | 50 | 52 | 54 | 56 | 58 | 60 | 62 | 64 |

TABLE 7
Nucleic Acid Sequence Identifiers of Selected Anti-PD-L1 × Anti-CD28 Bispecific
Antibodies

| | Anti-PD-L1 | | | | Anti-CD28 | | | | Common | | | |
|---|---|---|---|---|---|---|---|---|---|---|---|---|
| | Antigen-Binding | | | | Antigen-Binding | | | | Light Chain Variable | | | |
| Bispecific | Domain | | | | Domain | | | | Region | | | |
| Antibody | | HCDR | HCDR | HCDR | | HCDR | HCDR | HCDR | | LCDR | LCDR | LCDR |
| Designation | HCVR | 1 | 2 | 3 | HCVR | 1 | 2 | 3 | LCVR | 1 | 2 | 3 |
| REGN6192 | 1 | 3 | 5 | 7 | 9 | 11 | 13 | 15 | 17 | 19 | 21 | 23 |
| REGN6193 | 1 | 3 | 5 | 7 | 31 | 33 | 35 | 37 | 17 | 19 | 21 | 23 |
| REGN6194 | 41 | 43 | 45 | 47 | 49 | 51 | 53 | 55 | 57 | 59 | 61 | 63 |

TABLE 8
Amino acid and nucleotide sequences for full length immunoglobulin chains of
bispecific antibodies REGN6192, REGN6193 and REGN6194

| | HC (PD-L1) | | HC (CD28) | | LC (PD-L1 & CD28) | |
|---|---|---|---|---|---|---|
| Bispecific Antibody Designation | D | P | D | P | D | P |
| REGN6192 | 25 | 26 | 27 | 28 | 29 | 30 |
| REGN6193 | 25 | 26 | 39 | 40 | 29 | 30 |
| REGN6194 | 65 | 66 | 67 | 68 | 69 | 70 |

D = Nucleotide sequence of DNA encoding indicated sequence
P = amino acid of polypeptide for the indicated sequence
Numbers refer to SEQ ID NOs for the indicated sequence
HC is the full length heavy chain for the indicated antibody
LC is the full length light chain for the indicated antibody

Additional bispecific antibodies comprising one HCVR from a parental PD-L1 antibody and the other HCVR arm from a parental CD28 antibody may be made using the techniques described herein. The parental PD-L1 antibodies used to generate these additional anti-PD-L1×anti-CD28 bispecific antibodies have HCVR sequences described above in Table 1. The CD28 parental antibodies used to generate these additional anti-PD-L1×anti-CD28 bispecific antibodies have the amino acid sequences described above in Table 3 and in WO 2020/198009. These anti-PD-L1 and anti-CD28 binding domains (pairings) are shown below in Table 9.

TABLE 9
Summary of Parental Antibody Designations for HCVR Arms of
Additional Anti- PD-L1 x Anti-CD28 Bispecific Antibodies

| Anti-PD-L1 | Anti-CD28 |
|---|---|
| Antigen-Binding Domain | Antigen-Binding Domain |
| (Parental Antibody Designation and | (Parental Antibody Designation and |
| Sequences found in Table 1) | Sequences found herein in Table 3) |
| mAb9364 | mAb14193 |
| mAb9364 | mAb14216 |
| mAb9364 | mAb14226 |
| mAb9373 | mAb14193 |
| mAb9373 | mAb14216 |
| mAb9373 | mab14226 |

The bispecific antibodies described in the following examples consist of antigen-binding arms that bind to human hCD28 protein; and human PD-L1 (see, for example, Biacore binding data below). Exemplified bispecific antibodies contain a modified (chimeric) IgG4 Fc domain as set forth in U.S. Pat. No. 9,359,437.

The bispecific antibodies created in accordance with the present Example comprise two separate antigen-binding domains (i.e., binding arms). The first antigen-binding domain comprises a heavy chain variable region derived from an anti-CD28 antibody (“CD28-VH”), and the second antigen-binding domain comprises a heavy chain variable region derived from an anti-PD-L1 antibody (“PD-Li-VH”). Both the anti-PD-L1 and the anti-CD28 share a common light chain. The CD28-VH/PD-L1-VH pairing creates antigen-binding domains that are useful for targeting CD28 on T cells and PD-L1 on, for example, tumor cells and antigen presenting cells.

### Example 2: Characterization of Bispecific Antibodies that Bind CD28 and PD-L1 by Surface Plasmon Resonance

### Pd-L1 Kinetics:

Equilibrium dissociation constants (K_{D} values) for human PD-L1 expressed with a C-terminal myc-myc-hexahistidine tag (hPD-L1.mmH, SEQ ID NO: 71) binding to purified anti-PD-L1×CD28 antibodies were determined using a real-time surface plasmon resonance (SPR) biosensor technology using a Biacore S200 instrument. The CM5 Biacore sensor surface was derivatized by amine coupling with a monoclonal mouse anti-human Fc antibody. All Biacore binding studies were performed in a buffer composed of 0.01 M HEPES pH 7.4, 0.15M NaCl, 3 mM EDTA, 0.05% v/v Surfactant P20 (HBS-EP running buffer). Different concentrations of hPD-L1.mmH prepared in HBS-EP running buffer (ranging from 0.37 nM to 30 nM in 3-fold serial dilutions) were injected over the captured antibodies at a flow rate of 50 μL/minute. Antibody-reagent association was monitored for 5 minutes while dissociation in HBS-EP running buffer was monitored for 10 minutes. At the end of each cycle, the antibody capture surface was regenerated using a 12 sec injection of 20 mM phosphoric acid. All binding kinetics experiments were performed at 25° C.

### CD28 Kinetics:

Equilibrium dissociation constants (KD values) for human CD28 expressed with a C-terminal murine Fc tag (hCD28.mFc, SEQ ID NO: 72) binding to purified anti-PD-L1×CD28 antibodies were determined using a real-time surface plasmon resonance biosensor technology using a Biacore T200 instrument. The CM5 Biacore sensor surface was derivatized by amine coupling with a polyclonal goat anti-mouse antibody (anti-mFc, Cytiva). All Biacore binding studies were performed in a buffer composed of 0.01 M HEPES pH 7.4, 0.15M NaCl, 3 mM EDTA, 0.05% v/v Surfactant P20 (HBS-EP running buffer). Different concentrations of antibodies prepared in HBS-EP running buffer (ranging from 10 nM to 90 nM in 3-fold serial dilutions) were injected over the captured hCD28.mFc at a flow rate of 50 μL/minute. Antibody-reagent association was monitored for 4 minutes while dissociation in HBS-EP running buffer was monitored for 5 minutes. At the end of each cycle, the antibody capture surface was regenerated using a 40 sec injection of 10 mM glycine, pH 1.5. All binding kinetics experiments were performed at 25° C.

### Data Analysis:

The specific SPR-Biacore sensorgrams were obtained by a double referencing procedure. The double referencing was performed by first subtracting the signal of each injection over a reference surface (anti-hFc or anti-mFc) from the signal over the experimental surface (anti-hFc-captured anti-PD-L1×CD28 antibodies or anti-mFc-captured hCD28.mFc) thereby removing contributions from refractive index changes. In addition, running buffer injections were performed to allow subtraction of the signal changes resulting from the dissociation of captured antibodies or antigens from the coupled anti-hFc or anti-mFc surface. Kinetic association (kₐ) and dissociation (k_{d}) rate constants were determined by fitting the real-time sensorgrams to a 1:1 binding model using Scrubber v2.0c curve fitting software. Binding dissociation equilibrium constants (K_{D}) and dissociative half-lives (t½) were calculated from the kinetic rate constants as:

$${K}_{D} ( M ) = \frac{kd}{ka} , and t 1 / 2 ( min ) = \frac{ln ( 2 )}{60 * kd}$$

Biacore analysis showed that PD-L1×CD28 bound hPD-L1 with a K_{D} of ˜4.5E-11 to 1.2E-10 and hCD28 with a K_{D} of ˜4.7E-08 to 1.3E-08 (Tables 10 and 11).

TABLE 10
Kinetic and Equilibrium Binding Parameters of hPD-L1 to Surface-captured
anti-PD-L1 × CD28 Antibodies at 25° C.

| | | 30 nM | | | | |
|---|---|---|---|---|---|---|
| | mAb | hPD-L1.mmH | | | | |
| | Capture | Bind | | | | t1/2 |
| Ab PID | (RU) | (RU) | ka (1/M/s) | kd (1/s) | KD (M) | (min) |
| REGN6192 | 302.6 ± 1.0 | 34.9 | 6.00 E+05 | 6.18E−05 | 1.03E−10 | 186.8 |
| REGN6193 | 309.6 ± 0.9 | 44.2 | 5.48 E+05 | 6.37E−05 | 1.16E−10 | 181.4 |
| REGN6194 | 347.9 ± 1.3 | 62.8 | 1.11 E+06 | 4.97E−05 | 4.46E−11 | 232.5 |
| mAb9364 | 379.9 ± 0.8 | 112.2 | 5.71 E+05 | 7.42E−05 | 1.30E−10 | 155.7 |
| mAb9373 | 296.2 ± 1.8 | 103.1 | 9.77 E+05 | 5.58E−05 | 5.71E−11 | 206.9 |
| Isotype | 303.7 ± 0.5 | 0.5 | NB | NB | NB | NB |
| Control | | | | | | |

* NB: Non-binding

TABLE 11
Kinetic and Equilibrium Binding Parameters of hCD28 to Surface-captured
anti-PD-L1 × CD28 Antibodies at 25° C.

| | hCD28.mFc | 90 nM mAb | | | | t1/2 |
|---|---|---|---|---|---|---|
| Ab PID | Capture (RU) | Bind (RU) | ka (1/M/s) | kd (1/s) | KD (M) | (min) |
| REGN6192 | 69.2 ± 0.1 | 15.4 | 2.59E+04 | 3.42E−04 | 1.32E−08 | 33.8 |
| REGN6193 | 69.1 ± 0.1 | 21.6 | 3.59E+04 | 1.68E−03 | 4.69E−08 | 6.9 |
| REGN6194 | 69.0 ± 0.0 | 39.4 | 1.33E+05 | 3.26E−03 | 2.45E−08 | 3.5 |
| mAb9364 | 69.0 ± 0.1 | −0.9 | NB | NB | NB | NB |
| mAb9373 | 68.9 ± 0.1 | −1.3 | NB | NB | NB | NB |
| Isotype | 69.3 ± 0.1 | −4.3 | NB | NB | NB | NB |
| control | | | | | | |

* NB: Non-binding

### Example 3: Characterization of Bispecific PD-L1×CD28 Antibodies Binding to Cells Expressing PD-L1 or CD28

hPD-L1×CD28 bispecific antibody binding to cells was characterized using flow cytometry. Binding of the PD-L1 arm was tested using HEK293 cells, which were engineered to express PD-L1 (HEK293/hCD20/hPSMA/hPD-L1). Binding of the CD28 arm was assessed using Jurkat/NFkB-Luc cells, which express endogenous CD28. HEK293 cells, lacking both PD-Li and CD28 expression (HEK293/hCD20/hPSMA) were used to evaluate non-specific binding. Binding was detected by using a labeled secondary antibody and measuring fluorescence on a flow cytometer. Results are shown in Table 12.

### Cell Lines:

Jurkat/NFkB-Luc (ACL12421) were Jurkat cells stably transduced with a nuclear factor kappa-light-chain-enhancer of activated B cells (NF-κB)-luciferase reporter construct; the cell line was maintained in RPMI-1640+10% FBS+L-Glu/PS+1 μg/mL Puromycin. HEK293/hCD20/hPSMA/hPD-L1 (ACL11384) were HEK293/hCD20/hPSMA cells were stably transduced with a human programmed death ligand-1 construct. The cell line was maintained in DMEM High Glucose+10% FBS+L-Glu/PS+1 μg/mL Puromycin+500ug/mL G418. HEK293/hCD20/hPSMA (ACL11383) were HEK293 cells transfected with a hCD20 construct and selected with neomycin (G418), followed by flow sorting to obtain a cell line with high CD20 expression. Cells were then stably transduced with a human prostate-specific membrane antigen construct and then sorted for high expression of hPSMA. The cell line was maintained in DMEM High Glucose+10% FBS+L-Glu/PS+500ug/mL G418.

### Assay Set Up:

HEK293+/−PD-L1 cells were lifted with trypsin, washed and resuspended in stain buffer (2% FBS in PBS). Likewise, Jurkat/NFkB-Luc cells were centrifuged and resuspended in stain buffer. Cells were counted and 80 ul added to wells at 200,000 cells/well. Subsequently 20 ul of 5× antibody titrated from 100 nM to 6 pM (final concentration in well) in an 9-point 1:4 dose titration were added to cells. Cells and antibodies were incubated for 30 min on ice and then washed in stain buffer. Cells were resuspended in 2 μg/ml allophycocyanin (APC) conjugated goat-anti human secondary antibody. A secondary antibody alone control was included. Cells and secondary were incubated for 30 min on ice and then washed in stain buffer. After a subsequent wash with PBS (containing no FBS), cells were resuspended in viability dye (reconstituted in DMSO according to the manufaturer's protocol and diluted 1:1000 in PBS) and incubated for 30 min on ice. They were then washed with stain buffer and resuspended in 2% PFA and incubated overnight. After washing with stain buffer, they were filtered and analyzed by flow cytometry. EC₅₀ values of the antibodies were determined from a 4 parameter logistic equation over a 9-point dose response curve (including secondary only control) using GraphPad Prism software.

### Controls:

In addition to isotype controls, the following controls were used: Control 1: a bispecific antibody with one arm binding to CD28 (derived from parental antibody mAb14226) and the other arm binding to an unrelated antigen; Control 2: a bispecific antibody with one arm binding binding to CD28 (derived from parental antibody mAb14216) and the other arm binding to an unrelated antigen; and Control 3: a bispecific antibody with one arm binding binding to CD28 (derived from parental antibody mAb14193) and the other arm binding to an unrelated antigen.

### Binding on Jurkat/NFkB-Luc Cells:

Dose-dependent binding of the PD-L1×CD28 antibodies (REGN6192, REGN6193, and REGN6194) was observed in the presence of Jurkat/NFkB-Luc cells, with REGN6194 exhibiting the most potent and highest maximum binding. To note, only REGN6194 exhibited binding that could generate an EC50 value, as binding with REGN6192 and REGN6193 did not saturate at the highest tested concentration. Similar binding was observed for bispecific control antibodies (Control 1, Control 2, and Control 3), such that the antibody having the same CD28 arm as REGN6194 (Control 1), exhibited the most potent and greatest maximum binding. In comparison, CD28 bivalent control antibodies mAb14193, mAb14216, and mAb14226, corresponding to PD-L1×CD28 antibodies REGN6192, REGN6193, and REGN6194, respectively, exhibited more potent and greater maximum binding. PD-L1 bivalent control antibodies, mAb9364 and mAb9373, showed low maximum, but dose-dependent, binding on Jurkat/NFkB-Luc cells, indicating possible low endogenous expression of PD-L1 on these cells. Isotype control antibody did not bind Jurkat/NFkB-Luc cells.

### Binding on HEK293 and HEK293/PDL1 Cells:

Low, dose dependent binding of PD-L1×CD28 and PD-L1 bivalent controls was observed on HEK293 cells lacking PD-L1 overexpression, possibly suggesting a low level of endogenous PD-L1 expression. To note, max binding was not much higher than what was observed for isotype controls. On the contrary, for HEK293 cells engineered to overexpress hPD-L1, both PD-L1×CD28 and PD-L1 control antibodies displayed high maximum, dose-dependent binding in the low to sub-nanomolar range. CD28 bivalent and bispecific control antibodies did not bind HEK293 cells in a dose-dependent manner.

TABLE 12
Maximum binding and EC50 values of binding for antibodies

| | HEK298/hCD20/PSMA | | | HEK293/hCD20/PSMA/PDL1 | | | Jurkat/NFkB-Luc ch.1C11 | | |
|---|---|---|---|---|---|---|---|---|---|
| | | Max | Fold | | Max | Fold | | Max | Fold |
| Antibody | EC₅₀ [M] | [gMFI] | Change | EC₅₀ [M] | [gMFI] | Change | EC₅₀ [M] | [gMFI] | Change |
| REGN6192 | 2.3E−10 | 3.6E+03 | 3.3 | 2.5E−09 | 1.4 E+06 | 875.7 | NC | 1.0 E+04 | 24.1 |
| REGN6193 | 4.2E−10 | 4.3E+03 | 4.0 | 1.3E−09 | 1.3 E+06 | 846.8 | NC | 9.2 E+03 | 21.8 |
| REGN6194 | 1.2E−11 | 3.3E+03 | 3.1 | 1.7E−09 | 1.7 E+06 | 1066.7 | 7.5E−09 | 2.8 E+04 | 90.4 |
| mAb14226 | NC | 9.1E+03 | 8.3 | NC | 1.0 E+04 | 6.4 | 7.3E−10 | 4.3 E+04 | 101.4 |
| mAb14193 | ND | 1.1E+03 | 1.0 | ND | 4.7 E+03 | 3.0 | 2.1E−08 | 1.8 E+04 | 42.5 |
| mAb14216 | ND | 1.2E+03 | 1.0 | ND | 1.8 E+03 | 1.2 | 9.3E−09 | 2.5 E+04 | 60.3 |
| Control 2 | NC | 3.1E+03 | 2.9 | ND | 4.5 E+03 | 2.9 | NC | 7.4 E+03 | 17.7 |
| Control 3 | ND | 1.1E+03 | 1.0 | ND | 2.0 E+03 | 1.3 | 2.1E−08 | 1.0 E+04 | 24.8 |
| Control 1 | ND | 1.1E+03 | 1.0 | ND | 1.5 E+03 | 1.0 | 5.1E−09 | 4.2 E+04 | 99.2 |
| mAb9364 | NC | 3.1E+03 | 2.9 | 7.3E−10 | 6.2 E+05 | 400.9 | NC | 2.0 E+03 | 4.7 |
| mAb9373 | ND | 3.2E+03 | 3.0 | 62.E−10 | 9.5 E+05 | 609.3 | NC | 2.2 E+03 | 5.2 |
| Isotype Control | ND | 1.2E+03 | 1.1 | NC | 2.7 E+04 | 17.0 | N/D | 5.7 E+02 | 1.4 |

Geometric Mean Fluorescence Intensity (gMFI) values were plotted using GraphPad Prism and EC50 values of the antibodies were determined using a four-parameter, variable slope, non-linear regression equation over a 9-point dose-response curve, with a 1:4 titration of antibody ranging from 100 nM to 6 pM, and a no antibody condition.
Maximum gMFI, is the highest MFI along the dose response curve and the fold change is the maximum MFI divided by the MFI value from secondary antibody alone.
Abbreviations:
ND: Not Determined, as no dose dependent binding was observed;
NC: Not calculated because the data did not fit & 4-parameter logistic equation.

### Example 4: Activation of T Cells by PD-L1×CD28 Antibodies

Two signals, “signal 1” & “signal 2”, are required for proper T cell activation. “Signal 1” is induced by binding of the T cell receptor (TCR) on T cells to peptide-bound major histocompatibility complex (MHC) molecules on antigen presenting cells (APCs). “Signal 2” is provided by engaging co-stimulatory receptors on T-cells, such as CD28, with their ligands, such as cluster of differentiation 80 or 86 (CD80/CD86), on APC's (Martin et al. 1986; June et al. 1987; Harding et al. 1992). Therefore, activation of CD28 signaling provides a targeted approach to enhance existing TCR signaling.

PD-L1×CD28 bispecific antibodies are designed to mimic the natural ligands of CD28, by bridging PD-L1+target cells with CD28+ T cells, to provide “signal 2” in order to enhance the activation of T cells in the presence of a “signal 1” provided by a Tumor-associated antigen (TAA)×CD3.

### Characterization of T Cell Activation Using a Reporter Assay

The ability of PD-L1×CD28 bispecific antibodies to activate human primary T-cells by engaging PD-L1 on target cells and CD28 on T-cells, to deliver “signal 2”, was evaluated in an engineered reporter assay. In this assay, Jurkat cells (endogenously express CD28) are engineered to express the reporter gene luciferase under the control of the transcription factor NF-kB (NFkB-Luc). Target cells are HEK293 cells engineered to express CD20 and PSMA alone, or in combination with PD-L1. Reporter Jurkat cells are incubated with target cells (HEK293/hCD20/hPSMA and HEK293/hCD20/hPSMA/hPD-L1), as well as a bispecific CD20×CD3 antibody to provide “signal 1” and the ability of PD-L1×CD28 antibodies to specifically engage PD-L1 on target cells and facilitate CD28 clustering/activation of NFkB mediated luciferase production, was evaluated.

### Experimental Procedure:

One day before the experiment, Jurkat reporter cells were split to 5×10⁵ cells/mL in RPMI+10% FBS+Penicillin/Streptomycin/L-glutamine (P/S/G)+1 μg/ml Puromycin growth media. On the day of the experiment Jurkat/NFkB-luc cells were resuspended in assay media (RPMI supplemented with 10% FBS+P/S/G) and were added to 96-well white plates at a final concentration of 5×10⁴ cells/well. HEK293/hCD20/hPSMA and HEK293/hCD20/hPSMA/hPD-L1 were lifted with trypsin, washed and resuspended in assay media. Cells were added to the appropriate wells of 96-well white plates at a final concentration of 1×10⁴ cells/well. Constant CD20×CD3 was prepared in assay media and added to the appropriate wells at a final concentration of 0.1 nM. Subsequently, bispecific PD-L1×CD28, monospecific or bispecific controls or isotype controls were titrated 51.2 fM to 500 nM in a 1:5 dilution, the final point of the 12-point dilution containing no titrated antibody. All titrations were performed in duplicate and added to the appropriate wells. The antibody dilutions were all generated in assay media. Plates were incubated at 37° C. and 5% CO2 for 5 hours and then ONE-Glo luciferase substrate was added to each well according to manufacturer's instructions. The luciferase activity was recorded as a luminescence signal using the ENVISION plate reader and expressed as relative light units (RLU). The EC₅₀ values were determined by a 4-parameter logistic equation over a 12-point response curve using GraphPad Prism™. Signal recorded for the 12^{th} point on the dilution curve (no titrated antibody) was plotted at 10 fM. Maximal RLU is given as the mean max response detected within the tested dose range.

### Controls Used Included:

Control 4: a bispecific antibody with one arm binding to CD28 (derived from parental antibody mAb14226) and the other arm binding to an unrelated antigen; Control 5: a bispecific antibody with one arm binding to PD-L1 (derived from parental antibody mAb9373) and the other arm binding to an unrelated antigen; and Control 6: a bispecific antibody with one arm binding to PD-L1 (derived from parental antibody mAb9364) and the other arm binding to an unrelated antigen.

### Results:

In the presence of Jurkat/NFkB-Luc cells and target cells expressing CD20, the addition of CD20×CD3 led to increased baseline activity compared to conditions lacking CD20×CD3. Additionally, in the absence of CD20×CD3, bivalent CD28 antibody (mAb14226) led to a minor, dose dependent increase, in NFkB activity, which was greatly increased in the presence of ‘signal 1’ (CD20×CD3).

In the presence of HEK293/hCD20/hPSMA/hPD-L1 target cells and CD20×CD3 primary stimulation, the PD-L1×CD28 molecules (REGN6192, REGN6193, and REGN6194) led to a dose dependent increase in luciferase activity, while the isotype control antibodies did not result in an increase. In the absence of CD20×CD3 primary stimulation only the bivalent CD28 molecule (mAb14226) and one of the PD-L1×CD28 molecules (REGN6192) led to a slight increase in activity at the highest tested antibody concentrations, with no potency value assigned, due to lack of signal plateau.

In the presence of CD20×CD3 primary stimulation and HEK293/hCD20/hPSMA target cells lacking PD-L1, REGN6193 and Control 5 did not lead to an increase in NFkB activity, while REGN6192 and REGN6194 led to a slight increase at highest tested concentrations, albeit the response was weaker than the non-TAA×CD28′ control (Control 4) and an EC50 value could not be generated, due to lack of signal plateau. Additionally, isotype control antibodies did not result in any signal. In the absence of CD20×CD3 primary stimulation the PDL1×CD28 molecules did not generate a dose-response curve in the presence of HEK293/hCD20/hPSMA cells.

Luciferase activity and potency values of antibodies are shown in Table 13.

TABLE 13
Maximum luciferase activity and potency values of antibodies

| | HEK293/hCD20/ | | | | HEK293/hCD20/ | | | |
|---|---|---|---|---|---|---|---|---|
| | hPSMA + | | HEK293/hCD20/ | | hPSMA/hPD-L1 + | | HEK293/hCD20/ | |
| | CD20 × CD3 | | hPSMA | | CD20 × CD3 | | hPSMA/hPD-L1 | |
| | MAX | EC₅₀ | MAX | EC₅₀ | MAX | EC₅₀ | MAX | EC₅₀ |
| Antibodies | (RLU) | [M] | (RLU) | [M] | (RLU) | [M] | (RLU) | [M] |
| REGN6192 | 4.44E+05 | NC | 1.72E+04 | ND | 2.32E+06 | 2.43E−10 | 1.10E+05 | NC |
| REGN6193 | 2.17E+05 | ND | 1.42E+04 | ND | 2.73E+06 | 4.13E−10 | 1.95E+04 | ND |
| REGN6194 | 4.25E+05 | NC | 1.14E+04 | ND | 3.60E+06 | 2.80E−11 | 1.67E+04 | ND |
| Control 5 | 1.71E+05 | ND | 1.37E+04 | ND | 1.47E+05 | ND | 1.19E+04 | ND |
| Control 6 | 1.78E+05 | ND | 1.14E+04 | ND | 1.45E+05 | ND | 1.73E+04 | ND |
| mAb9373 | 1.81E+05 | ND | 1.10E+04 | ND | 1.55E+05 | ND | 1.11E+04 | ND |
| mAb9364 | 1.80E+05 | ND | 4.44E+04 | ND | 1.65E+05 | ND | 5.71E+04 | ND |
| Control 4 | 5.94E+05 | 4.01E−08 | 1.44E+04 | ND | 4.92E+05 | 7.28E−08 | 1.33E+04 | ND |
| mAb14226 | 6.49E+05 | 5.14E−11 | 7.32E+04 | 5.69E−09 | 5.96E+05 | 8.75E−11 | 6.08E+04 | NC |
| Isotype | 1.73E+05 | ND | 1.09E+04 | ND | 1.34E+05 | ND | 1.00E+04 | ND |
| control 1 | | | | | | | | |
| Isotype | 1.72E+05 | ND | 1.11E+04 | ND | 1.42E+05 | ND | 1.02E+04 | ND |
| Control 2 | | | | | | | | |

Abbreviations:
ND: Not Determined because no dose dependent response was observed;
NC: Not calculated because the data did not fit a 4-parameter logistic equation.

### Characterization of T Cell Activation Using Human T Cells

The ability of PD-L1×CD28 bispecific antibodies to activate human primary T-cells by engaging PD-L1 and CD28 to deliver “signal 2”, as determined by IL2 release, was evaluated in the presence of a human embryonic kidney cancer cell line engineered to express hCD20, hPSMA, and hPD-L1 (HEK293/hCD20/hPSMA/hPD-L1) using a bispecific CD20×CD3 antibody to serve as “signal 1.” HEK293 cells expressing only hCD20 and hPSMA were included as a control to measure activity that may occur in the absence of PD-L1 on APC's.

### Experimental Procedures:

Human peripheral blood mononuclear cells (PBMCs) were isolated from a healthy donor leukocyte pack from Precision for Medicine (Donor 555060) using the following protocol: 15 mL of density gradient medium (FicollPaque Plus) was added to a 50 mL tube. A leukocyte pack was diluted 1:1 with PBS+2% FBS. The diluted leukocyte pack was added on top of density gradient medium. Tubes were centrifuged at room temperature at 400G for 30 minutes with brake off. The upper plasma layer was removed and discarded. The mononuclear cell layer at the plasma-density gradient medium interface, was removed and retained. PBS+2% FBS was added to the collected mononuclear cell layer and centrifuged at 300G for 8 minutes. The supernatant was discarded and the resultant PBMC resuspended in appropriate medium. CD4⁺ T-cells were isolated using CD4⁺ T-cell isolation kits (Miltenyi Biotech.) and following manufacturer's recommend instructions.

### IL2 Release Assay:

Enriched CD4⁺ T-cells, resuspended in stimulation media, were added into 96-well round bottom plates at a concentration of 1×10⁵ cells/well. Growth-arrested HEK293/hCD20/hPSMA or HEK293/hCD20/hPSMA/hPD-L1 were added to CD4⁺ T-cells at a final concentration of 1×10⁴ cells/well. Following addition of cells, a constant of 0.2 nM CD20×CD3 or its matched isotype control was added to wells containing HEK293/hCD20/hPSMA or HEK293/hCD20/hPSMA/hPD-L1. Subsequently, bispecific PD-L1×CD28, CD28 bivalent, PD-L1 bivalent, bispecific controls, or isotype control antibodies were titrated from 15 pM to 100 nM in a 1:3 dilution and added to wells. The final point of the 10-point dilution contained no titrated antibody (CD20×CD3 or isotype control antibody only). Plates were incubated for 72 hours at 37° C., 5% CO₂ and 5 μL total supernatant was removed and used for measuring IL2. The amount of cytokine in assay supernatant was determined using AlphaLisa kits from PerkinElmer following the manufacturer's protocol. The cytokine measurements were acquired on Perkin Elmer's multilabel plate reader Envision and values were reported as RFU (relative fluorescent units). All serial dilutions were tested in duplicate. The EC₅₀ values of the antibodies were determined from a four-parameter logistic equation over a 10-point dose-response curve using GraphPad Prism™ software with the 10^{th} point (no titrated antibody) being represented by 5.1 pM. Maximal IL2 is given as the mean max response detected within the tested dose range.

### Controls Used:

As disclosed in the Examples above.

### Results:

In the presence of T-cells and CD20×CD3, target cells expressing PD-L1 led to decreased baseline activity compared to target cells lacking PD-L1. Additionally, in the absence of CD20×CD3, none of the tested antibodies led to dose dependent increases in IL-2 release.

In the presence of HEK293/hCD20/hPSMA/hPD-L1 target cells and CD20×CD3 primary stimulation, the PD-L1×CD28 (REGN6192, REGN6193 and REGN6194) molecules led to a dose dependent increase in IL2 release, while the isotype control antibodies did not result in an increase. REGN6194 led to the highest maximum cytokine release among all molecules tested. An increase in IL-2 was observed for ×CD28 bispecific controls (Control 1, Control 2 and Control 3), however only at highest tested concentrations and EC50 values could not be generated, as the signal did not plateau. Bivalent CD28 antibody, mAb14226, also led to a dose dependent increase in IL-2 release, in the presence of CD2×CD3, while the other CD28 bivalent antibodies mAb14193 and mAbR14216 did not.

In the presence of CD20×CD3 primary stimulation and target cells lacking PD-L1, REGN6192 and REGN6194 led to an increase in 1.2 release in a dose dependent manner, comparable to respective ×CD28 controls (Control 2 and Control 3, respectively) and to a much weaker extent compared to the response observed with high PD-L1 expressing target cells. Bivalent CD28 molecules led to slight dose dependent increases in IL-2 release in the presence of CD20×CD3 antibody.

IL2 release and potency values of antibodies are shown in Table 14.

TABLE 14
Maximum IL2 release and potency values of antibodies

| | HEK293/ | | HEK293/ | | HEK293/hCD20/ | | HEK293/ | |
|---|---|---|---|---|---|---|---|---|
| | hCD20/hPSM | | hCD20/h | | hPSMA/hPD-L1 + | | hCD20/hP | |
| | A + CD20 × CD3 | | PSMA | | CD20 × CD3 | | SMA/hPD-L1 | |
| | MAX | EC₅₀ | MAX | EC₅₀ | MAX | EC₅₀ | MAX | EC₅₀ |
| Antibodies | (RLU) | [M] | (RLU) | [M] | (RLU) | [M] | (RLU) | [M] |
| REGN6192 | 2.52E+04 | 1.64E−08 | 3.87E+03 | ND | 1.44E+05 | 1.04E−10 | 2.69E+03 | ND |
| REGN6193 | 1.26E+04 | ND | 4.25E+03 | ND | 1.41E+05 | 1.44E−10 | 2.76E+03 | ND |
| REGN6194 | 3.37E+04 | NC | 5.48E+03 | NC | 1.65E+05 | NC | 2.87E+03 | ND |
| mAb14226 | 6.44E+04 | 1.16E−10 | 2.91E+03 | ND | 3.88E+04 | 2.67E−09 | 2.86E+03 | ND |
| mAb14193 | 3.36E+04 | 2.49E−08 | 3.11E+03 | ND | 4.72E+03 | ND | 3.07E+03 | ND |
| mAb14216 | 2.56E+04 | 9.54E−09 | 3.04E+03 | ND | 3.62E+03 | ND | 2.86E+03 | ND |
| Control 2 | 5.04E+04 | NC | 3.10E+03 | ND | 2.73E+04 | ND | 3.55E+03 | ND |
| Control 3 | 2.77E+04 | 2.44E−08 | 2.94E+03 | ND | 3.98E+03 | ND | 3.23E+03 | ND |
| Control 1 | 3.51E+04 | 4.17E−08 | 3.38E+03 | ND | 1.12E+04 | ND | 4.30E+03 | ND |
| mAb9364 | 8.98E+03 | ND | 4.36E+03 | ND | 1.09E+04 | ND | 3.68E+03 | ND |
| mAb9373 | 9.08E+03 | ND | 4.53E+03 | ND | 6.96E+03 | ND | 3.64E+03 | ND |
| Isotype | 1.01E+04 | ND | 3.28E+03 | ND | 2.83E+03 | ND | 3.89E+03 | ND |
| control | | | | | | | | |

Abbreviations:
ND: Not Determined because a dose dependent response was not observed;
NC: Not calculated because the data did not fit a 4-parameter logistic equation.

### Example 5: Blocking of PD-L1 Binding to PD1 by PD-L1×CD28 Antibodies

### Characterization of PD-L1 Blocking by ELISA

ELISA-based blocking assays were developed to determine the ability of PD-L1×CD28 bispecific antibodies to block the binding of human programmed death-ligand 1 (hPD-L1) to human programmed cell death protein 1 (hPD1).

The human PD-L1 recombinant protein used in the experiments was comprised of the hPD-L1 extracellular domain (amino acids F19-T239) expressed with the Fc portion of mouse IgG2a at the C-terminus (amino acids E98-K330) (hPD-L1-mFc, accession #NP_054862.1). The human PD1 protein was comprised of the hPD1 extracellular domain (amino acids L25-V170; C93S) expressed with the Fc portion of the human IgG1 at C-terminus (amino acids D104-K330) (hPD1-hFc, accession #NP_005009.2).

In the PD-L1 blocking assay, hPD1-hFc protein (2pg/ml in PBS) was coated onto a 96-well microtiter plate overnight at 4° C. Nonspecific binding sites were subsequently blocked using a 0.5% (w/v) solution of BSA in PBS. In a separate 96-well microtiter plate, a fixed amount of 0.7 nM hPD-L1-mFc was bound for one hour with PD-L1×CD28 bispecific antibodies, their parental bivalent anti-PD-L1, anti-CD28 or relevant human isotype antibodies at dilutions ranging from 1.7 pM to 100 nM in PBS+0.5% BSA. The fixed concentration of hPD-L1-mFc was selected to be near the concentration that generated 50% of the maximal binding (EC₅₀ value) of the hPD1 plate. The hPD-L1-mFc antibody complexes were then transferred to the hPD1 coated plate. After one hour of incubation at room temperature, the plates were washed, and plate-bound hPD-L1-mFc protein was detected with horseradish peroxidase (HRP) conjugated goat anti-mouse Fcγ fragment specific antibody. The plates were then developed using TMB substrate solution (BD Biosciences) according to the manufacturer's recommended procedure and the absorbance at 450 nm was measured on a SpectraMax 13× plate reader.

Binding data were analyzed using a sigmoidal (four-parameter logistic) dose-response model with GraphPad Prism™ software. The calculated IC₅₀ value, defined as the concentration of antibody required to block 50% of hPD-L1-mFc binding to plate-coated hPD1-hFc, was used as an indicator of blocking potency. The percent blocking of tested antibodies at the highest tested concentration of 100 nM was calculated based on the formula shown below:

$$\% Blocking = 100 - ( \frac{[ Experimental {Signal}_{( 100 nM Ab )} - Background {Signal}_{( buffer )} ]}{[ Maximum {Signal}_{( 0.7 nM hPD - L 1 )} - Background {Signal}_{( buffer )} ]} ) \times 100$$

Antibodies that blocked greater than 50% of binding at the highest concentration tested were classified as blockers and their IC₅₀ values were reported.

### Results:

For the blocking of PD-L1 interaction with PD1, three PD-L1×CD28 bispecific antibodies (REGN6192, REGN6193 and REGN6194) displayed concentration dependent blocking of hPD-L1 binding to hPD1 with 97% to 99% blocking at the highest antibody concentration tested (100 nM). The IC₅₀ values for these bispecific antibodies ranged from 0.2 nM to 1.2 nM (Table 15). Their parental bivalent PD-L1 antibodies (mAb9364 and mAb9373) inhibited hPD-L1 binding to hPD1 with a similar percent blocking of 99% and IC₅₀ values of 0.49 nM and 0.52 nM respectively. All parental bivalent CD28 (mAb14226, mAb14193 and mAb14216) antibodies, and the isotype control antibodies showed no or low blocking of hPD-L1 binding to hPD1, with percentage blocking ranging from −1% to 19%, classifying these antibodies as non-blockers.

TABLE 15
Summary of PD-L1xCD28 Bispecific Abs and Their Parental
Abs Blocking Human PD-L1 binding to Human PD1

| | Antibody blocking 0.7 nM hPD-L1- | |
|---|---|---|
| | mFc binding to hPD1-hFc | |
| | | % Blocking at |
| Antibody ID | IC₅₀ (M) | 100 nM mAb |
| REGN6192 | 1.2E−09 | 97 |
| REGN6193 | 8.2E−10 | 98 |
| REGN6194 | 2.7E−10 | 98 |
| mAb9373 | 4.9E−10 | 99 |
| mAb9364 | 5.2E−10 | 99 |
| mAb14226 | NBL | 19 |
| mAb14193 | NBL | 6 |
| mAb14216 | NBL | 1 |
| Isotype control 1 | NBL | 1 |
| Isotype control 2 | NBL | 1 |
| Isotype control 3 | NBL | 4 |

NBL: Non-blocking, % blocking is less than or equal to 50%
NA: Not applicable

### Characterization of PD-L1 Blocking in Cells

Characterization of PD-L1×CD28 bispecific antibodies in a PD-L1 blocking assay was carried out using WSU-DLCL2, WSU-DLCL2/hPD-L1, and Jurkat/AP1-luc/hPD1 cells. This experiment investigated whether PD-L1 bivalent, PD-L1×CD28, bispecific control antibodies or relevant isotype controls, can block PD-L1 from interacting with PD1. CD20×CD3 bispecific antibody provides ‘signal 1’ by engaging CD20 on WSU-DLCL2 target cells (endogenous expression) and CD3 on Jurkat/AP1-luc/hPD1 cells, resulting in an increase in AP-1-Luc activity, observed by an increased luminescent signal. WSU-DLCL2 cells engineered to express PD-L1 will result in decreased AP-1 activity. The ability of PD-L1 antibodies to block PD1 interaction with PD-L1, resulting in recovery of the luciferase signal, was evaluated.

Cell lines: Jurkat/AP1-Luc/hPD1 (ACL8709) was generated from Jurkat cells stably transduced with a human programmed cell death protein 1 construct; the cell line was maintained in RPMI-1640+10% FBS+L-Glu/PS+1ug/mL Puromycin. WSU-DLCL2/hPD-L1 (ACL17386) was generated from WSU-DLCL2 cells stably transduced with a human programmed death ligand-1 construct; the cell line was maintained in RPMI-1640+10% FBS+L-Glu/PS+1ug/mL Puromycin. WSU-DLCL2 cells (HCT883) were maintained in RPMI-1640+10% FBS+L-Glu/PS.

### Experimental Set-Up:

One day before the experiment, Jurkat reporter cells were split to 5×10⁵ cells/ml in RPMI+10% FBS+Penicillin/Streptomycin/L-glutamine (P/S/G)+1 μg/ml Puromycin growth media. Jurkat/AP1-Luc/PD1 were resuspended in assay media (RPMI supplemented with 10% FBS+P/S/G) and added to 96-well white plates at a concentration of 5×10⁴ cells/well. Antigen presenting cells with or without PD-L1 expression (WSU-DLCL2/PD-L1 or WSU-DLCL2, respectively) were also resuspended in assay media and added to the plates at a concentration of 2.5×10⁴ cells/well. A bispecific CD20×CD3 antibody was added to all wells at a constant concentration of 1 nM. Subsequently, PD-L1×CD28, relevant monospecific or bispecific controls or isotype control antibodies were titrated 7.6 pM to 500 nM in a 1:4 dilution, the final point of the 10-point dilution containing no titrated antibody (CD20×CD3 antibody constant only). All titrations were performed in duplicate and added to the appropriate wells. The antibody dilutions were all generated in assay media. Plates were incubated at 37° C. and 5% CO2 for 5 hours and then ONE-Glo luciferase substrate was added to each well according to manufacturer's instructions. The luciferase activity was recorded as a luminescence signal using the ENVISION plate reader and expressed as relative light units (RLU). The EC₅₀ values were determined by a 4-parameter logistic equation over a 10-point response curve using GraphPad Prism™. Signal recorded for 10^{th} point on the dilution curve was plotted at 1.9 pM. Maximal RLU is given as the mean max response detected within the tested dose range. An anti-PD1 antibody (REGN2810; cemiplimab, LIBTAYO®) was used in the experiment along with other controls.

### Results:

In the presence of CD20×CD3 antibody, WSU-DLCL2 cells expressing PD-L1 exhibited a decrease in AP-1 activity, in comparison to PD-L1 negative WSU-DLCL2 cells.

In the absence of PD-L1 expression on the WSU-DLCL2 cells, PD-L1 bispecific as well as bivalent antibodies and controls did not impact AP-1 activity.

In the presence of PD-L1 expression on the WSU-DLCL2 cells, PD-L1 bispecific, PD-L1 bivalent, REGN2810, and ×PD-L1 bispecific control (Control 5 and Control 6) antibodies led to a dose dependent rescue of lumincesence signal, which was suppressed by the PD1 and PD-L1 interaction. Isotype controls did not lead to an increase in AP-1 activity (Table 16).

TABLE 16
Maximum RLU (of AP-1 activity) and Potency values of Antibodies

| | WSU-DLCL2 | | WSU-DLCL2/hPD-L1 | |
|---|---|---|---|---|
| | MAX | | MAX | |
| Antibodies | (RLU) | EC50 [M] | (RLU) | EC50 [M] |
| REGN6192 | 1.19E+06 | ND | 1.19E+06 | 1.83E−10 |
| REGN6193 | 1.22E+06 | ND | 1.49E+06 | 2.02E−10 |
| REGN6194 | 1.27E+06 | ND | 1.44E+06 | 1.77E−12 |
| Control 5 | 1.24E+06 | ND | 1.05E+06 | 2.09E−10 |
| Control 6 | 1.26E+06 | ND | 1.10E+06 | 8.74E−10 |
| mAb9373 | 1.26E+06 | ND | 1.09E+06 | 1.71E−12 |
| mAb9364 | 1.24E+06 | ND | 1.04E+06 | 6.51E−12 |
| REGN2810 | 1.23E+06 | ND | 1.10E+06 | 2.90E−10 |
| Isotype Control 1 | 1.24E+06 | ND | 2.88E+05 | ND |
| Isotype Control 2 | 1.19E+06 | ND | 2.66E+05 | ND |
| Isotype Control 3 | 1.19E+06 | ND | 2.49E+05 | ND |

Abbreviations: ND: Not Determined because no dose-dependent response was observed;
NC: Not calculated because the data did not fit a 4-parameter logistic equation.

### Example 6: Enhancement of Killing of MUC16+ Cells by Bispecific PD-L1×CD28 Antibodies in Combination with Muc16×CD3 Antibody

The costimulatory PD-L1×CD28 bispecific antibody REGN6194 was tested for its ability to enhance the cytotoxic potency of REGN4019, a bispecific antibody targeting CD3 and MUC16 (WO 2018/067331). MUC16 is a tumor antigen expressed on the surface of OVCAR-3 tumor cells engineered to overexpress hPD-L1. A control PD-L1×4-1 BB bsAb (Comparator 1) comprising the variable regions of “CD137-009-HC7LC2” and “PD-L1-547” (WO 2019/025545) was included in the experiment.

In order to monitor the killing of MUC16+ cells by flow cytometry in the presence of hPBMC and a combination of a MUC16×CD3 with a PD-L1×CD28 bispecific antibody (bsAbs), OVCAR-3/hPD-L1 cells were labeled with 1 uM of the fluorescent tracking dye Violet Cell Tracker. After labeling, cells were plated overnight at 37° C. Separately, human PBMCs were plated in supplemented RPMI media at 1×106 cells/mL and incubated overnight at 37° C. in order to enrich for lymphocytes by depleting adherent macrophages, dendritic cells, and some monocytes. The next day, target cells were co-incubated with adherent cell-depleted naïve PBMC (Effector/Target cell 4:1 ratio), a serial dilution of MUC16×CD3 bispecific antibody REGN4019 or a ×CD3 bispecific isotype control (control×CD3) (concentration range: 66.7 nM to 150 pM) and a fixed concentration of the PD-L1×CD28 costimulatory bispecific antibody at 2.5ug/ml (16.7 nM) for 72 hours at 37° C. Cells were removed from cell culture plates using Trypsin-EDTA dissociation buffer, and analyzed by Flow cytometry on a BD Celesta cytometer. For Flow cytometry analysis, cells were stained with a dead/live Near IR Reactive (Invitrogen) dye. 2E04 counting beads were added to each well immediately before FACS analysis. 1 E04 beads were collected for each sample. For the assessment of specificity of killing, cells were gated on live Violet labeled populations. Percent of live population was recorded and used for the calculation of survival.

T cell activation and upregulation of the PD1 marker were assessed by incubating cells with directly conjugated antibodies to CD2, CD4, CD8, CD25 and PD1, and by reporting the percent of activated (CD25+/CD8+, CD25+/CD4+) T cells and PD1+/CD4+, PD1+/CD8+ T cells out of total T cells (CD2+).

The supernatant of the assay wells from the human PBMC assay were assessed for Th1/Th2 cytokine release using the BD cytometric bead array human kit and following the manufacturer protocol.

Results: In the absence of a targeting ×CD3 bispecific such as MUC16×CD3 REGN4019, REGN6194 as well as Comparator 1 were inert. On the other hand, a fixed concentration of the PD-L1×CD28 bsAb REGN6194 successfully enhanced the cytotoxic potency of REGN4019 in the presence of human PBMC against OVCAR-3/hPD-L1 cells, and the cells were killed in a dose-dependent manner. REGN6194 was more potent than Comparator 1 at enhancing REGN4019 cytotoxicity. Besides the observed target-cell lysis enhancement, T cell activation was increased, with upregulation of CD25 and PD1 expression on CD4+ and CD8+ T cells (see Table 17).

TABLE 17
EC50 of killing and T cell activation by bispecific PD-L1 × CD28 antibodies in
combination with Muc16 × CD3 antibody (REGN4019)

| | | Cytotoxicity | T cell activation, EC50 [M] for: | | | |
|---|---|---|---|---|---|---|
| | Co-stimulatory | | CD4+/ | CD8+/ | CD4+/ | CD8+/ |
| Signal 1 | signal | EC50[M] | CD25+ | CD25+ | PD1+ | PD1+ |
| MUC16 × CD3 | — | 6.00E−10 | 1.63E−09 | 1.28E−09 | 7.57E−10 | 1.43E−09 |
| (REGN4019) | | | | | | |
| MUC16 × CD3 | Comparator 1 | 3.25E−10 | 3.50E−10 | 3.25E−10 | 1.50E−10 | 4.51E−11 |
| (REGN4019) | | | | | | |
| MUC16 × CD3 | REGN6194 | 5.74E−11 | 5.73E−11 | 9.34E−11 | 4.74E−11 | 5.22E−11 |
| (REGN4019) | | | | | | |
| control × CD3 | — | N/C | N/C | N/C | N/C | N/C |
| control × CD3 | Comparator 1 | N/C | N/C | N/C | N/C | N/C |
| control × CD3 | REGN6194 | N/C | N/C | N/C | N/C | N/C |

N/C: Not calculable

The supernatants from the cytotoxicity experiment were assessed for cytokine release. REGN4019's cytotoxic potency was associated with accumulation of IFNg in the media, and IFNg media accumulation was enhanced when REGN419 was combined with PD-L1×CD28. Combining REGN419 with REGN6194 (PD-L1×CD28) resulted in the higher level of cytokine accumulation in media than Comparator 1 (see Table 18).

TABLE 18
Cytokine released induced by bispecific PD-L1xCD28 antibodies in
combination with Muc16xCD3 antibody (REGN4019)

| | | Cytokines accumulation in media | |
|---|---|---|---|
| Signal 1 | Co-stimulatory signal | IFNg, pg/ml | IFNg, EC50 |
| MUC16xCD3 | | 31 | 4.05E−09 |
| MUC16xCD3 | REGN6194 | 1974 | 2.76E−10 |
| MUC16xCD3 | Comparator 1 | 138 | 1.89E−09 |
| controlxCD3 | | 10 | N/C |
| controlxCD3 | REGN6194 | 10 | N/C |
| controlxCD3 | Comparator 1 | 10 | N/C |

N/C: Not calculable

### Example 7: Potent Anti-Tumor Efficacy of a Bispecific PD-L1×CD28 Antibody in MC38/hPDL1 Tumor Model

This example relates to an in vivo study demonstrating the efficacy of a bispecific PD-L1×CD28 antibody in treating tumors in a murine model of colon cancer. Mice were humanized for PD1, PD-L1, CD28, and CD3γδε (CD3-gamma-delta-epsilon), wherein the mouse genes were knocked out and replaced with their human homologues (hPD1/hPD-L1/hCD28/hCD3 mice). Tumors were generated from MC38 colon carcinoma cells that were engineered to knock out mouse PD-L1 and overexpress human PD-L1 (M38-hPDL1-mPDL1KO cells). The following antibodies were used in this study: PD-L1×CD28 (REGN6194), PD-L1×BetV1 (a bispecific control with one arm binding PD-L1 and the other arm binding the unrelated antigen BetV1), BetV1×CD28 (a bispecific control with one arm binding CD28 and the other arm binding BetV1), and Isotype control.

On Day 0, 6 to 7 hPD1/hPD-L1/hCD28/hCD3 mice per group were implanted with M38-hPDL1-mPDL1KO cells. When tumors reached an average tumor volume of approximately 100 mm³ on Day 8, antibodies were administered on Days 9, 13, 16, 20, and 22 at a dose of 10 mg/kg. Tumors were measured approximately twice per week until end of experiment at Day 60.

### Results:

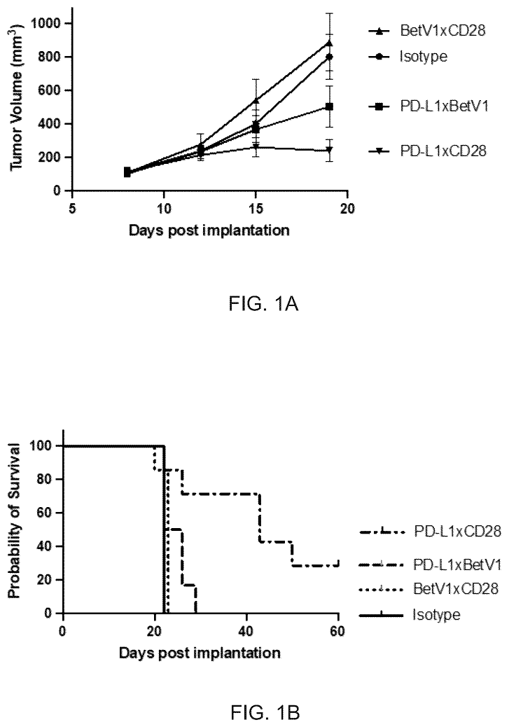

REGN6194 potently controlled tumor growth and increased survival compared to Isotype control or either bispecific control antibody (FIGS. 1A-1 B). For example, at Day 19, the average volume of tumors treated with Isotype control or BetV1×CD28 was approximately 800 mm³. In contrast, the average volume of REGN6194-treated tumors was significantly lower, i.e., approximately 200 mm³ (FIG. 1A). Additionally, mice administered REGN6194 exhibited dramatically improved survival as compared to mice administered Isotype control or either bispecific control antibody—e.g., by Day 60, 30% of mice administered REGN6194 had survived, while no control mice survived past Day 30 (FIG. 1 B). REGN6194 is referred to as “PD-L1×CD28” in FIG. 1B. In summary, the bispecific PD-L1×CD28 antibody was effective at inhibiting tumor growth and supporting survival.

### Example 8: Potent Anti-Tumor Efficacy of Bispecific PD-L1×CD28 Antibodies Alone and in Combination with an Anti-PD1 Antibody in MC38/hPDL1 Tumor Model

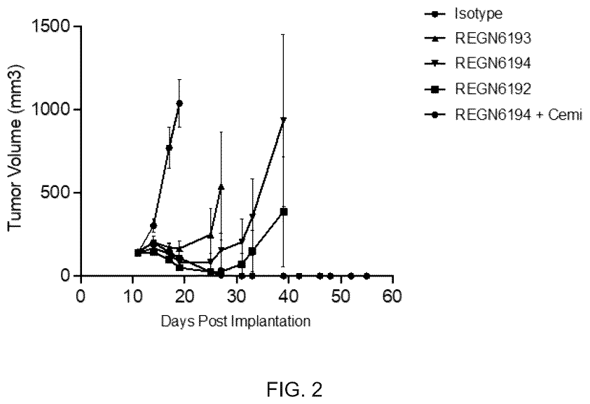

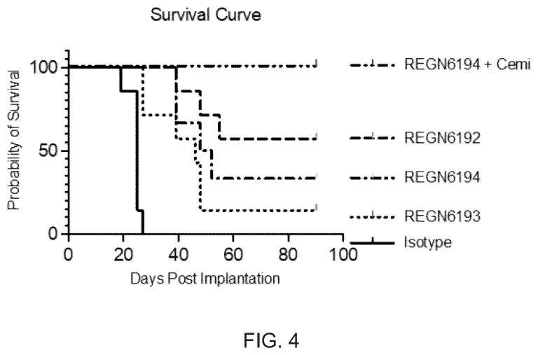

This example relates to an in vivo study demonstrating the efficacy of bispecific PD-L1×CD28 antibodies, both as monotherapy and in combination with the anti-PD1 antibody cemiplimab, in treating tumors in a murine model of colon cancer. Mice were humanized for PD1, PD-L1, CD28, and CD3γδε (CD3-gamma-delta-epsilon), wherein the mouse genes were knocked out and replaced with their human homologues (hPD1/hPD-L1/hCD28/hCD3 mice), and tumors were generated from MC38 colon carcinoma cells that were engineered to knock out mouse PD-L1 and overexpress human PD-L1 (M38-hPDL1-mPDL1KO cells). The following antibodies were used in this study: three PD-L1×CD28 antibodies (REGN6192, REGN6193, and REGN6194), cemiplimab, and Isotype control. In FIGS. 2 and 4, cemiplimab is referred to as “Cemi.”

On Day 0, 6 to 7 hPD1/hPD-L1/hCD28/hCD3 mice per group were implanted with MC38-hPDL1-mPDL1KO cells. When tumors reached an average tumor volume of approximately 140 mm³ on Day 11, antibodies were administered on Days 11, 14, 17, 19 and 25 at a dose of 10 mg/kg. Tumors were measured approximately twice per week until end of experiment at Day 90.

### Results:

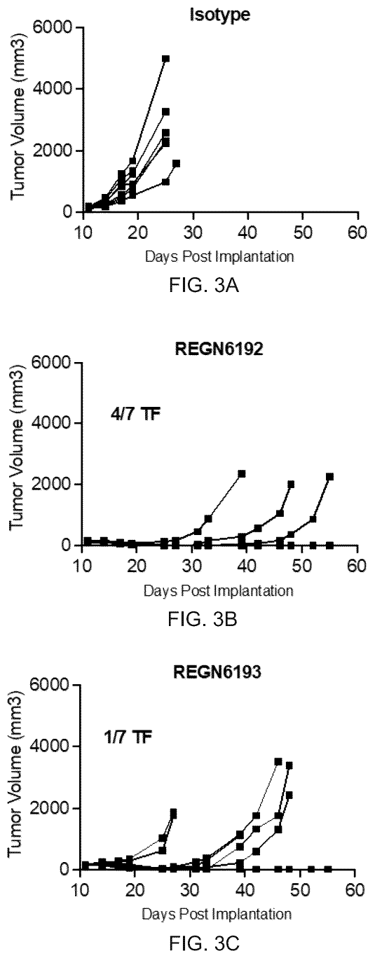

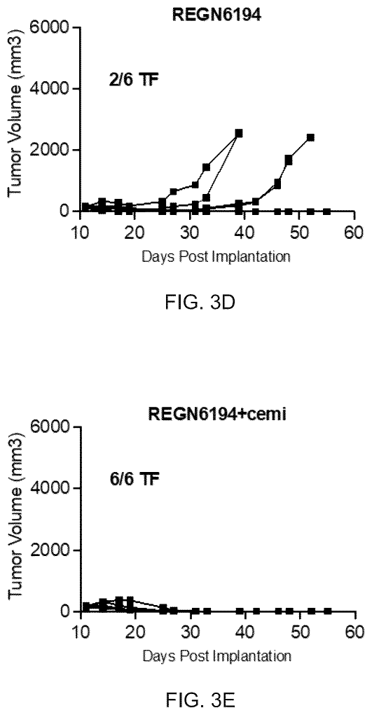

All bispecific PD-L1×CD28 antibodies exhibited potent anti-tumor efficacy compared to Isotype control (FIG. 2), and this effect was enhanced when combined with cemiplimab (FIGS. 3A-3E). For example, at Day 19, when Isotype control treated-tumors reached a volume of over 1000 mm³, the average volumes of tumors treated with REGN6192, REGN6193, or REGN6194 were all significantly lower, i.e., below 200 mm³ (FIG. 2). Furthermore, while none of the mice administered Isotype control became tumor free (FIG. 3A) by Day 55, 4 out of 7 mice administered REGN6192 (FIG. 3B), 1 out of 7 mice administered REGN6193 (FIG. 3C), and 2 out of 6 mice administered REGN6194 (FIG. 3D) were tumor free at Day 55. Combination therapy of REGN6194 and cemiplimab resulted in greater anti-tumor efficacy than REGN6194 monotherapy, with 6 out of 6 mice tumor free at Day 55 (FIG. 3E). Furthermore, while average tumor volume eventually increased when treated with REGN6194 alone, approaching 1000 mm³ by Day 38; when treated with REGN6194 in combination with cemiplimab, average tumor size dropped dramatically to 0 mm³ by Day 25 and remained so until end of experiment at Day 90 and was associated with long term survival (FIGS. 2 and 4). This complete and durable tumor regression with the combination therapy of a PD-L1×CD28 bispecific antibody and anti-PD1 was a remarkable result.

### Example 9: Potent Control of Tumor Growth by Treatment with a Bispecific PD-L1×CD28 Antibody in Combination with an Anti-PD1 Antibody in B16F10/hPDL1 Tumor Model

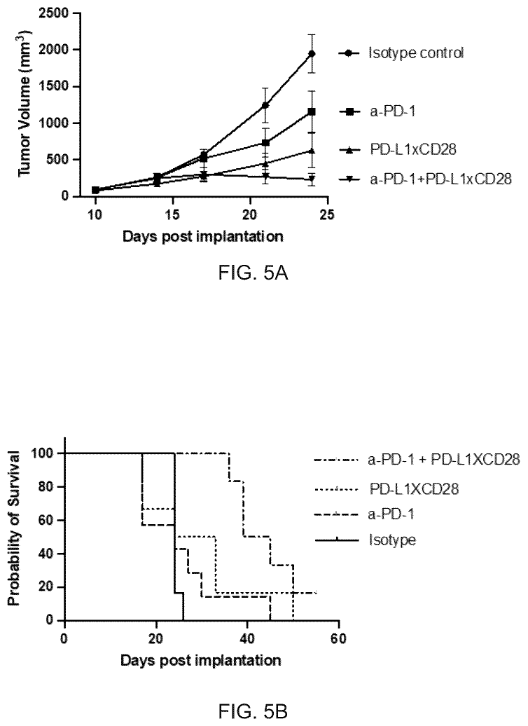

This example relates to an in vivo study demonstrating the efficacy of a bispecific PD-L1×CD28 antibody in combination with the anti-PD1 antibody cemiplimab, in treating tumors in a murine model of melanoma known to be resistant to a-PD1 therapy. Mice were humanized for PD1, PD-L1, CD28, and CD3γδε (CD3-gamma-delta-epsilon), wherein the mouse genes were knocked out and replaced with their human homologues (hPD1/hPD-L1/hCD28/hCD3 mice), and tumors were generated from B16F10 cells that were engineered to knock out mouse PD-L1 and overexpress human PD-L1 (B1i6F10-hPDL1-mPDL1KO cells). The following antibodies were used in this study: PD-L1×CD28 (REGN6194), a-PD-1 (cemiplimab), and Isotype control. In FIGS. 5A and 5B, REGN6194 is referred to as “PD-L1×CD28,” and cemiplimab is referred to as “a-PD-1.”

On Day 0, 6 hPD1/hPD-L1/hCD28/hCD3 mice per group were implanted with B16F10-hPDL1-mPDL1KO cells. Tumors reached an average tumor volume of approximately 100 mm³ on Day 10, and antibodies were administered on Days 10, 14, 16, 21, and 24 at a dose of 10 mg/kg. Tumors were measured approximately twice per week until Day 39. At end of experiment (Day 55), tumor-free mice were identified.

Results: REGN6194 administered monotherapy shows significant anti-tumor efficacy compared to isotype control and monotherapy a-PD1 cemiplimab in the B16F10/hPD-L1 tumor model (FIGS. 5A-5B). Interestingly, REGN6194 administered in combination with cemiplimab potently controlled tumor growth and increased survival compared to administration of either REGN6194 or cemiplimab alone (FIGS. 5A-5B). For example, at Day 24, while the average volume of tumors treated with Isotype control was approximately 2000 mm³, the average volume of tumors treated with the combination of REGN6194 and cemiplimab was significantly lower (i.e., less than 250 mm³). In comparison, also at Day 24, tumors treated with cemiplimab monotherapy exhibited an average tumor volume of approximately 1200 mm³, and tumors treated with REGN6194 monotherapy exhibited an average tumor volume of approximately 600 mm³ (FIG. 5A). In addition, while 1 out of 6 mice receiving combination therapy were tumor free at Day 55, none of the mice in the other treatment groups were tumor free at Day 55. Also, survival of mice administered the combination therapy of REGN6194 and cemiplimab was dramatically better than survival of mice administered either REGN6194 or cemiplimab as monotherapy. At Day 30, all mice receiving the combination therapy had survived, while only 50% of the mice administered REGN6194 and less than 20% of the mice administered cemiplimab as monotherapy had survived (FIG. 5B). This data shows potent tumor control with the combination therapy comprising PD-L1×CD28 and anti-PD1 to treat B16F10 tumors.

### Example 10: Lack of Induction of Cytokine Release in Response to Administration of a Bispecific PD-L1×CD28 Antibody Alone or in Combination with an Anti-PD1 Antibody

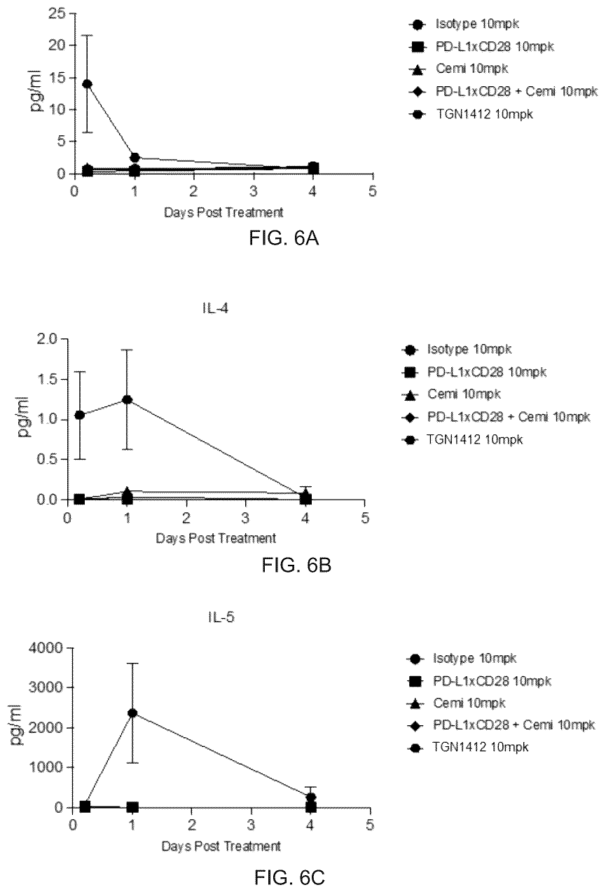

This example relates to an in vivo study demonstrating the improved cytokine expression profile in response to PD-L1×CD28 monotherapy or PD-L1×CD28 in combination with an anti-PD1 antibody in comparison to the anti-CD28 superagonist TGN1412. Mice were humanized for PD1, PD-L1, CD28, and CD3γδε (CD3-gamma-delta-epsilon), wherein the mouse genes were knocked out and replaced with their human homologues (hPD1/hPD-L1/hCD28/hCD3γδε (CD3-gamma-delta-epsilon) mice), and tumors were generated from parental M38 cells. The following antibodies were used in this study: PD-L1×CD28 (REGN6194), anti-PD1 (cemiplimab), the anti-CD28 superagonist TGN1412, and Isotype control. In FIGS. 6A-6C, REGN6194 is referred to as “PD-L1×CD28”, and cemiplimab is referred to as “Cemi.”

On Day 0, hPD1/hPD-L1/hCD28/hCD3 mice were implanted with M38 cells. Tumors reached an average tumor volume of approximately 100 mm³ on Day 7, and antibodies were administered on Days 7, 10, 14, 18, and 21 at a dose of 10 mg/kg. The concentrations of three different cytokines were measured from blood samples collected at 4, 24, and 96 hours after the first antibody administration on Day 7.

### Results:

While TGN1412 induced high levels of release of IL-2, IL-5, and IL-4, REGN6194 did not induce cytokine release when administered as a monotherapy or when administered in combination with cemiplimab (FIGS. 6A-6C).

### Example 11: Enhancement of Efficacy of the Combination Therapy of MAGE-A4×CD3 and Anti-PD1 when Combined with PD-L1×CD28 in SK-MEL-37 Tumor Model

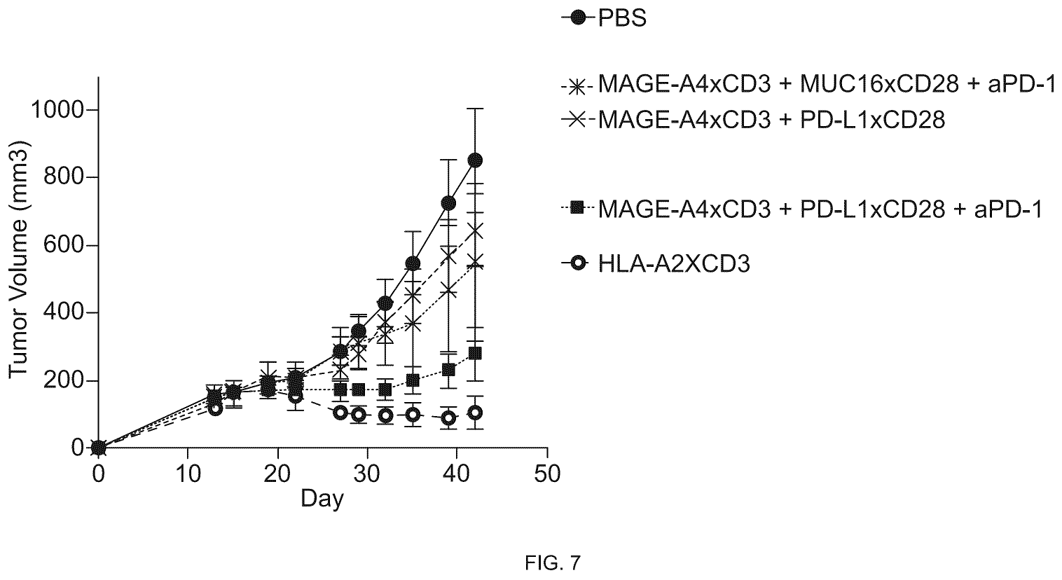

This example relates to an in vivo study demonstrating the ability of PD-L1×CD28 to improve the efficacy of a combination therapy comprising a TAA×CD28 bispecific antibody (i.e., a bispecific antibody with one arm binding a tumor specific antigen (TAA) and one arm binding CD28) and an anti-PD1 antibody. Mice used in this study were a highly immunodeficient strain NSG™ (null for NOD, SCID, and IL-2Rγ) with human peripheral blood mononuclear cells (hPBMC) introduced as a source of human immune cells. Tumors were generated from the human skin cancer cell line, SK-MEL-37, which have low copy numbers of the TAA MAGE-A4 and endogenous levels of human PD-L1. The following antibodies were used in this study: three bispecific antibodies (PD-L1×CD28 (REGN6194), MAGE-A4×CD3, and MUC16×CD28 as an unrelated TAA bispecific control); anti-PD1 (cemiplimab); and HLA-A2×CD3. In FIG. 7, REGN6194 is referred to as “PD-L1×CD28,” and cemiplimab is referred to as “Cemi.” Antibodies were administered to mice in the doses indicated in Table 19 and in the combinations as indicated in Table 20.

TABLE 19
Doses of Antibody Administrated

| Antibody | Dose (mg/kg) |
|---|---|
| MAGE-A4xCD3 | 0.04 |
| anti-PD1 (cemiplimab) | 4 |
| PD-L1xCD28 (REGN6194) | 4 |
| MUC16xCD28 | 4 |
| HLA-A2xCD3 | 4 |

TABLE 20
Combinations of Antibodies Administered

| Group | Combination |
|---|---|
| 1 | PBS |
| 2 | MAGE-A4xCD3 + anti-PD1 (cemiplimab) + MUC16xCD28 |
| 3 | MAGE-A4xCD3 + anti-PD1 (cemiplimab) + |
| | PD-L1xCD28 (REGN6194) |
| 4 | MAGE-A4xCD3 + PD-L1xCD28 (REGN6194) |
| 5 | HLA-A2xCD3 |

On Day 0, NSG mice were implanted with 5×10⁶ SK-MEL-37 cells. Tumors reached an average tumor volume of approximately 200 mm³ on Day 8, at which time the mice were engrafted with 4×10⁶ hPBMC. Antibodies were administered on Days 18, 21, 26, 29, and 33. Tumors were measured approximately twice per week from Days 13-43.

### Results:

Mice administered the combination therapy comprising MAGE-A4×CD3, cemiplimab, and REGN6194 showed potent control of tumor growth, with average tumor volume less than 300 mm³ at Day 43 (FIG. 7). In contrast, mice administered a combination therapy comprising MAGE-A4×CD3, cemiplimab, and the bispecific-control antibody MUC16×CD28 showed significantly inferior control of tumor growth, with average tumor volumes of approximately 650 mm³ at Day 43 (FIG. 7). Thus, the triple-combination therapy comprising PD-L1×CD28, MAGE-A4×CD3, and anti-PD1 was approximately 50% more effective than the double-combination therapy of MAGE-A4×CD3 and anti-PD1 at controlling tumor growth.

### Example 12: Dependency of Anti-Tumor Efficacy of a Bispecific PD-L1×CD28 Antibody on the Proportion of Human PD-L1⁺ Cells in Tumor

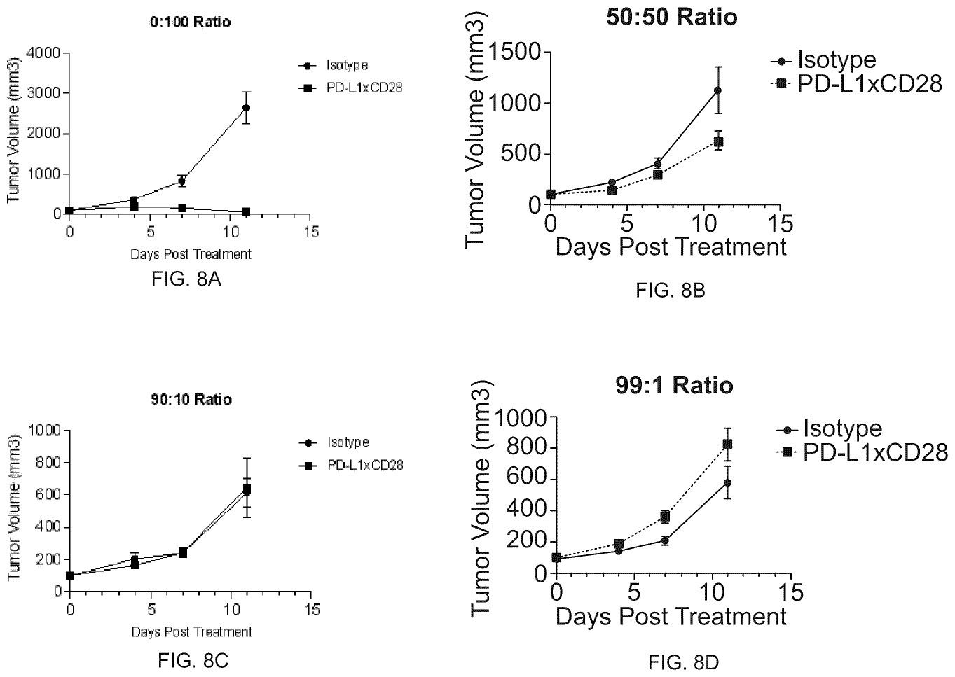

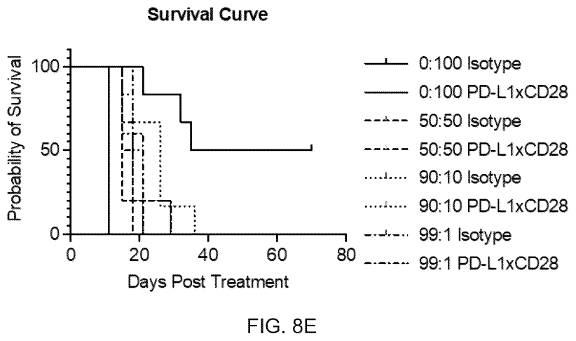

This example relates to an in vivo study demonstrating that tumors with a higher ratio of human PD-L1⁺ cells to human PD-L1⁻ cells are more effectively treated with REGN6194. Mice were humanized for PD1, PD-L1, CD28, and CD3γδε (CD3-gamma-delta-epsilon), wherein the mouse genes were knocked out and replaced with their human homologues (hPD1/hPD-L1/hCD28/hCD3 mice), and tumors were generated from mixtures of MC38 colon carcinoma cells that were engineered to knock out mouse PD-L1 and overexpress human PD-L1 (M38-hPDL1-mPDL1KO cells) and parental M38 cells, which express mouse PD-L1, not human PD-L1, i.e., human PD-L1⁻. The following antibodies were used in this study: PD-L1×CD28 (REGN6194) and Isotype control. In FIGS. 8A-8E, REGN6194 is referred to as “PD-L1×CD28.”

After hPD1/hPD-L1/hCD28/hCD3 mice were implanted with ratios of MC38 parental cells to MC38-hPDL1-mPDL1KO cells of 0:100, 50:50, 90:10, or 99:1 and tumor volumes reached about 100 mm³, the mice were administered 10 mg/kg REGN6194 or Isotype control on Day 0. Tumors were measured on Days 4, 7, and 11, and survival was assessed until end of experiment at 10 weeks post-treatment.

### Results:

REGN6194 monotherapy demonstrated modest anti-tumor efficacy on tumors derived from hPD-L1⁻ cells: hPD-L1⁺ cells at a ratio of 50:50 (FIG. 8B), but efficacy was lost on tumors derived from hPD-L1⁻ cells: hPD-L1⁺ cells at a ratio of 90:10 (FIG. 8C) and tumors derived from hPD-L1⁻ cells: hPD-L1⁺ cells at a ratio of 99:1 (FIG. 8D). Survival of mice with tumors derived from hPD-L1⁻ cells: hPD-L1⁺ cells at a ratio of 50:50 treated with REGN6194 was significantly greater than mice with tumors derived from hPD-L1⁻ cells: hPD-L1⁺ cells at a ratio of 90:10 or 99:1 (FIG. 8E).

### Example 13: Potent Growth Control of Tumors in Absence of hPD-L1 Expression by Combination Treatment of PD-L1×CD28 with Anti-PD1

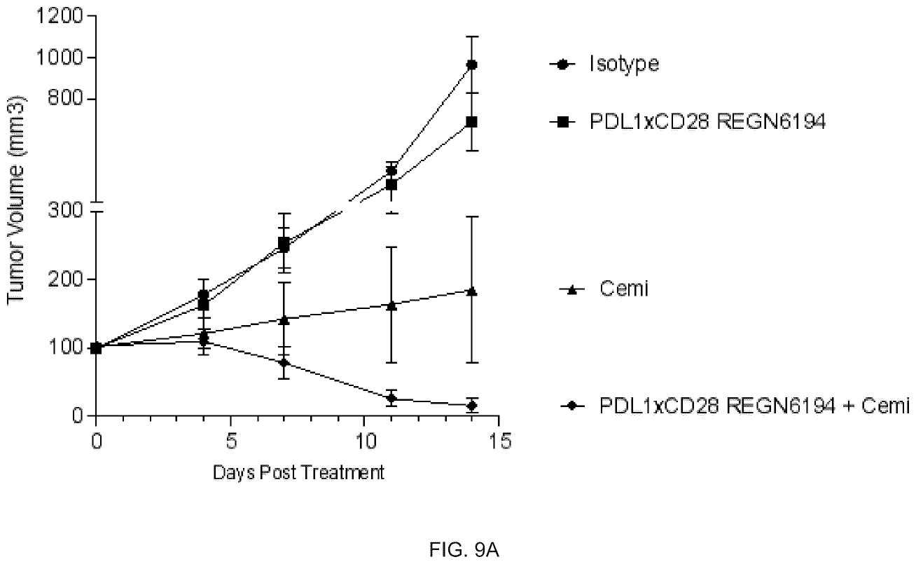

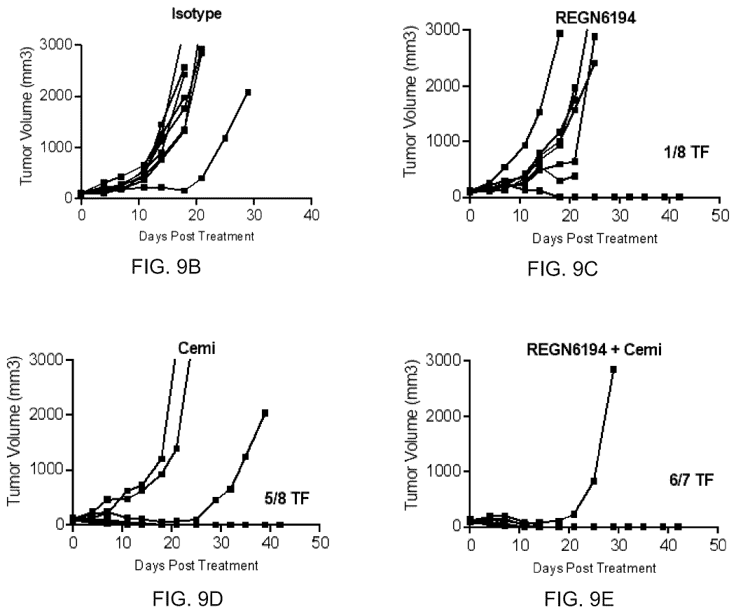

This example relates to an in vivo study demonstrating that tumors lacking human PD-L1 expression are effectively treated by the combination of PD-L1×CD28 and cemiplimab, indicating a role for PD-L1 expression in non-tumor cells. Mice were humanized for PD1, PD-L1, CD28, and CD3γδε (CD3-gamma-delta-epsilon), wherein the mouse genes were knocked out and replaced with their human homologues (hPD1/hPD-L1/hCD28/hCD3 mice), and tumors were generated from parental M38 cells, which express mouse PD-L1, not human PD-L1. The following antibodies were used in this study: PD-L1×CD28 (REGN6194), cemiplimab, and Isotype control. In FIGS. 9A-9E, REGN6194 is referred to as “PD-L1×CD28,” and cemiplimab is referred to as “Cemi.”

After hPD1/hPD-L1/hCD28/hCD3 mice were implanted with parental M38 cells and tumor volumes reached an average of approximately 100 mm³ (determined as DO), antibodies were administered on Days 0, 4, 7, 11, and 14 at a dose of 10 mg/kg. Tumors were measured approximately twice per week until end of experiment at 3 weeks post-treatment.

### Results:

Combination therapy comprising REGN6194 and cemiplimab potently controlled tumor growth as compared to either REGN6194 or cemiplimab monotherapy (FIGS. 9A-9E). For example, on Day 14 post-treatment, the average volume of tumors treated with the combination of REGN6194 and cemiplimab was dramatically controlled to under 15 mm³ (FIG. 9A). In comparison, on Day 14 post-treatment, the average tumor volume of mice administered Isotype control alone was approximately 1000 mm³, the average tumor volume of mice administered REGN6194 monotherapy was approximately 690 mm³, and the average tumor volume of mice administered cemiplimab monotherapy was approximately 185 mm³ (FIG. 9A). Additionally, of mice administered the combination therapy of REGN6194 and cemiplimab, 6 out of 7 were tumor free at end of experiment (4 weeks post-implantation), while only 5 out of 8 mice administered cemiplimab monotherapy were tumor free at end of experiment. Thus, the combination therapy of PD-L1×CD28 and anti-PD1 was effective at controlling the growth of tumors without human PD-L1 expression, which indicates a role for PD-L1 in non-tumor cells, such as antigen-presenting cells.

### Example 14: Dose-Response Efficacy and Formal pK of REGN6194

This prophetic example relates to in vivo studies demonstrating the dose-response efficacy in treating tumors in a murine model of colon cancer and formal pK of bispecific PD-L1×CD28 antibody (REGN6194), alone and in combination with the anti-PD1 antibody cemiplimab.

PD-L1×CD28 Monotherapy: Mice are humanized for PD1, PD-L1, CD28, and CD3γδε (CD3-gamma-delta-epsilon), wherein the mouse genes are knocked out and replaced with their human homologues (hPD1/hPD-L1/hCD28/hCD3 mice), and tumors are generated from MC38 colon carcinoma cells that are engineered to knock out mouse PD-L1 and overexpress human PD-L1 (M38-hPDL1-mPDL1KO cells). The following antibodies are used in this study: PD-L1×CD28 (REGN6194) and isotype control.

On Day 0, 7 to 8 hPD1/hPD-L1/hCD28/hCD3 mice per group are implanted with M38-hPDL1-mPDL1KO cells. Tumors reach an average tumor volume of 75-100 mm³ on Day 7, when mice are randomized into 6 groups according to tumor size, and antibodies are administered intraperitoneally (IP) on Days 11, 14, 21, and 28, at doses of 0.1, 1, 5, and 10 mg/kg for REGN6194, and 10 mg/kg for isotype control. Tumor size and body weight are measured, and survival is assessed twice per week. Blood is collected on Days 7, 11, and 14 both immediately before and 4 hours after antibody administration and on Days 21 and 28, and serum cytokines are measured and FACS analysis is performed.

### Results:

It is expected that REGN6194 will control tumor growth without loss of body weight and will increase survival as well. Effective anti-tumor activity is expected to be achieved even at low doses.

### Combination Therapy of PD-L1×CD28 and Cemiplimab:

Mice are humanized for PD1, PD-L1, CD28, and CD3γδε (CD3-gamma-delta-epsilon), wherein the mouse genes are knocked out and replaced with their human homologues (hPD1/hPD-L1/hCD28/hCD3 mice), and tumors are generated from MC38 colon carcinoma cells that are engineered to knock out mouse PD-L1 and overexpress human PD-L1 (M38-hPDL1-mPDL1KO cells). The following antibodies are used in this study: PD-L1×CD28 (REGN6194), anti-PD1 (cemiplimab), and isotype controls for each of REGN6194 and cemiplimab.

On Day 0, 6-8 hPD1/hPD-L1/hCD28/hCD3 mice per group are implanted with M38-hPDL1-mPDL1KO cells. Tumors reach an average tumor volume of 100-150 mm³ on Day 9, when mice are randomized into 7 groups according to tumor size, and antibodies are administered intraperitoneally (IP) on Days 13, 16, 23, and 30. Combination therapy comprises doses of 0.1, 1, 5, or 10 mg/kg for REGN6194 and 10 mg/kg for cemiplimab. REGN6194 monotherapy comprises either REGN6194 at 5 mg/kg and the isotype control for cemiplimab at 10 mg/kg or REGN6194 at 10 mg/kg and the isotype control for cemiplimab. Cemiplimab monotherapy comprises the isotype control for REGN6194 at 10 mg/kg and cemiplimab at 10 mg/kg. Both isotype control antibodies are administered to the control group. Tumor size and body weight are measured, and survival is assessed twice per week. Blood is collected on Days 9, 13, and 16 both immediately before and 4 hours after antibody administration. On Days 23 and 30, blood is collected only immediately before antibody administration. Blood samples are processed by measuring serum cytokine levels and performing FACS analysis.

### Results:

It is expected that the combination therapy of REGN6194 and cemiplimab will lead to greater control of tumor growth and increased survival than either antibody monotherapy. It is also expected that there will be no loss of body weight as a result of the administration of combination therapy. It is also expected that effective anti-tumor activity will be achieved with REGN6194 and cemiplimab combination therapy at a lower dose than the dose required to achieve effective anti-tumor activity with REGN6194 monotherapy.

Treatment of Tumors with the Combination of PD-L1×CD28 and Cemiplimab Compared to a Combination of Bispecific Control Antibodies:

Mice are humanized for PD1, PD-L1, CD28, and CD3γδε (CD3-gamma-delta-epsilon), wherein the mouse genes are knocked out and replaced with their human homologues (hPD1/hPD-L1/hCD28/hCD3 mice), and tumors are generated from MC38 colon carcinoma cells that are engineered to knock out mouse PD-L1 and overexpress human PD-L1 (M38-hPDL1-mPDL1KO cells). The following antibodies are used in this study, all at a dose of 10 mg/kg: PD-L1×CD28 (REGN6194); an unrelated bispecific-control antibody, PD-L1×BetV1; anti-PD1 (cemiplimab); an isotype-control antibody for cemiplimab; and one isotype-control antibody appropriate for both bispecific antibodies.

On Day 0, 6-8 hPD1/hPD-L1/hCD28/hCD3 mice per group are implanted with M38-hPDL1-mPDL1KO cells. Tumors reach an average tumor volume of 130-150 mm³ on Day 7, when mice are randomized into 6 groups according to tumor size, and antibodies are administered by IP injection on Days 7, 11, and 14. Combination therapy comprises REGN6194 and cemiplimab. The combination-therapy control comprises PD-L1×BetV1 and cemiplimab. Monotherapies consist of (i) REGN6194 and the isotype-control antibody for cemiplimab, (ii) PD-L1×BetV1 and the isotype-control antibody for cemiplimab, and (iii) the bispecific isotype-control antibody and cemiplimab. The negative control consists of the two isotype-control antibodies. Tumor size and body weight are measured, and survival is assessed twice per week. Blood is collected on Day 9 at 4 hours and 24 hours post-injection. On Days 13, 15, and 21 blood is collected only immediately before antibody administration. Blood samples are processed by measuring serum cytokine levels and performing FACS analysis.

### Results:

It is expected that combination therapy of REGN6194 and cemiplimab will control tumor growth without loss of body weight and will increase survival more effectively than the combination of PD-L1×BetV1 and cemiplimab, and more effectively than any of REGN6194, PD-L1×BetV1, or cemiplimab monotherapy.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims. All patents, applications and non-patent publications mentioned in this specification are incorporated herein by reference in their entireties.

1. An isolated bispecific antigen-binding molecule comprising:
(a) a first antigen-binding domain that specifically binds human CD28, with a K_{D} of less than about 3×10⁻⁸M as measured by surface plasmon resonance at 25° C.; and
(b) a second antigen-binding domain that specifically binds a human programmed death-ligand 1 (PD-L1) with a K_{D} of less than about 2×10⁻¹⁰ M as measured by surface plasmon resonance at 25° C.

2. The isolated bispecific antigen-binding molecule of claim 1, wherein the bispecific antigen-binding molecule binds to the surface of human T cells with an EC₅₀ of less than about 2×10⁻⁸ M as measured by an in vitro FACS binding assay.

3. The isolated bispecific antigen-binding molecule of claim 1, wherein the bispecific antigen-binding molecule binds to the surface of a cell expressing PD-L1 with an EC₅₀ of less than about 3×10⁻⁹ M as measured by an in vitro FACS binding assay.

4. The isolated bispecific antigen-binding molecule of claim 1, wherein the bispecific antigen-binding molecule blocks PD-L1 binding to PD-1 with an IC₅₀ of less than about 1.3 nM as measured by an ELISA-based blocking assay.

5. The isolated bispecific antigen-binding molecule of claim 1, wherein the bispecific antigen-binding molecule, in combination with a bispecific MUC16×CD3 antibody mediates in vitro T cell killing of OVCAR-3 cells expressing PD-L1 with an EC₅₀ of less than about 10⁻¹⁰ M.

6. The isolated bispecific antigen-binding molecule of claim 1, wherein the first antigen-binding domain comprises:
(a) three heavy chain complementarity determining regions (HCDR1, HCDR2 and HCDR3) contained within a heavy chain variable region (HCVR) comprising the amino acid sequence selected from the group consisting of SEQ ID NOs: 50, 32 and 10, or a variant thereof, and
(b) three light chain complementarity determining regions (LCDR1, LCDR2 and LCDR3) contained within a light chain variable region (LCVR) comprising the amino acid sequence selected from the group consisting of SEQ ID NOs: 58 and 18, or a variant thereof.

7. The isolated bispecific antigen-binding molecule of claim 6, comprising a HCDR1 comprising the amino acid sequence selected from the group consisting of SEQ ID NOs: 52, 34 and 12, a HCDR2 comprising the amino acid sequence selected from the group consisting of SEQ ID NOs: 54, 36 and 14, and a HCDR3 comprising the amino acid sequence selected from the group consisting of SEQ ID NOs: 56, 38 and 16.

8. The isolated bispecific antigen-binding molecule of claim 6, comprising a LCDR1 comprising the amino acid sequence selected from the group consisting of SEQ ID NOs: 60 and 20, a LCDR2 comprising the amino acid sequence selected from the group consisting of SEQ ID NOs: 62 and 22, and a LCDR3 comprising the amino acid sequence selected from the group consisting of SEQ ID NO: 64 and 24.

9. The isolated bispecific antigen-binding molecule of claim 7, wherein the first antigen-binding domain comprises a HCVR comprising the amino acid sequence of SEQ ID NO: 50, or a variant thereof, and a LCVR comprising the amino acid sequence of SEQ ID NO: 58, or a variant thereof.

10. The isolated bispecific antigen-binding molecule of claim 7, wherein the first antigen-binding domain comprises a HCVR comprising the amino acid sequence of SEQ ID NO: 32, or a variant thereof, and a LCVR comprising the amino acid sequence of SEQ ID NO: 18, or a variant thereof.

11. The isolated bispecific antigen-binding molecule of claim 7, wherein the first antigen-binding domain comprises a HCVR comprising the amino acid sequence of SEQ ID NO: 10, or a variant thereof, and a LCVR comprising the amino acid sequence of SEQ ID NO: 18, or a variant thereof.

12. The isolated bispecific antigen-binding molecule of claim 1, wherein the second antigen-binding domain comprises:
(a) three heavy chain complementarity determining regions (HCDR1, HCDR2 and HCDR3) contained within a heavy chain variable region (HCVR) comprising the amino acid sequence selected from the group consisting of SEQ ID NOs: 42 and 2, or a variant thereof, and
(b) three light chain complementarity determining regions (LCDR1, LCDR2 and LCDR3) contained within a light chain variable region (LCVR) comprising the amino acid sequence selected from the group consisting of SEQ ID NOs: 58 and 18, or a variant thereof.

13. The isolated bispecific antigen-binding molecule of claim 12, wherein the second antigen-binding domain comprises:
(a) a HCDR1 comprising the amino acid sequence of SEQ ID NO: 44 or SEQ ID NO: 4;
(b) a HCDR2 comprising the amino acid sequence of SEQ ID NO: 46 or SEQ ID NO: 6; and
(c) a HCDR3 comprising the amino acid sequence of SEQ ID NO: 48 or SEQ ID NO: 8.

14. The isolated bispecific antigen-binding molecule of claim 12, wherein the second antigen-binding domain comprises a LCDR1 comprising the amino acid sequence selected from the group consisting of SEQ ID NOs: 60 and 20, a LCDR2 comprising the amino acid sequence selected from the group consisting of SEQ ID NOs: 62 and 22, and a LCDR3 comprising the amino acid sequence selected from the group consisting of SEQ ID NO: 64 and 24.

15. The isolated bispecific antigen-binding molecule of claim 14, wherein the second antigen-binding domain comprises:
(a) HCDR1, HCDR2, HCDR3 domains, respectively, comprising the amino acid sequences of SEQ ID NOs: 44, 46, 48; and LCDR1, LCDR2, LCDR3 domains, respectively, comprising the amino acid sequences of SEQ ID NOs: 60, 62, 64; or
(b) HCDR1, HCDR2, HCDR3 domains, respectively, comprising the amino acid sequences of SEQ ID NOs: 4, 6, 8; and LCDR1, LCDR2, LCDR3 domains, respectively, comprising the amino acid sequences of SEQ ID NOs: 20, 22, 24.

16. The isolated bispecific antigen-binding molecule of claim 15, wherein the second antigen-binding domain comprises:
(a) a HCVR comprising the amino acid sequence of SEQ ID NO: 42, or a variant thereof, and a LCVR comprising the amino acid sequence of SEQ ID NO: 58, or a variant thereof; or
(b) a HCVR comprising the amino acid sequence of SEQ ID NO: 2, or a variant thereof, and a LCVR comprising the amino acid sequence of SEQ ID NO: 18, or a variant thereof.

17. An isolated bispecific antigen-binding molecule, comprising:
(a) a first antigen-binding domain that specifically binds human CD28 wherein the first antigen-binding domain comprises HCDR1, HCDR2, HCDR3 domains, respectively, comprising the amino acid sequences of SEQ ID NOs: 52,54, 56, and LCDR1, LCDR2, LCDR3 domains, respectively, comprising the amino acid sequences of SEQ ID NOs: 60,62,64; and
(b) a second antigen-binding domain that specifically binds human PD-L1 wherein the second antigen-binding domain comprises HCDR1, HCDR2, HCDR3 domains, respectively, comprising the amino acid sequences of SEQ ID NOs: 44,46,48, and LCDR1, LCDR2, LCDR3 domains, respectively, comprising the amino acid sequences of SEQ ID NOs: 60, 62, 64.

18. An isolated bispecific antigen-binding molecule, comprising:
(a) a first antigen-binding domain that specifically binds human CD28 wherein the first antigen-binding domain comprises HCDR1, HCDR2, HCDR3 domains, respectively, comprising the amino acid sequences of SEQ ID NOs: 34, 36, 38, and LCDR1, LCDR2, LCDR3 domains, respectively, comprising the amino acid sequences of SEQ ID NOs: 20, 22, 24; and
(b) a second antigen-binding domain that specifically binds human PD-L1 wherein the second antigen-binding domain comprises HCDR1, HCDR2, HCDR3 domains, respectively, comprising the amino acid sequences of SEQ ID NOs: 4, 6, 8, and LCDR1, LCDR2, LCDR3 domains, respectively, comprising the amino acid sequences of SEQ ID NOs: 20, 22, 24.

19. An isolated bispecific antigen-binding molecule, comprising:
(a) a first antigen-binding domain that specifically binds human CD28 wherein the first antigen-binding domain comprises HCDR1, HCDR2, HCDR3 domains, respectively, comprising the amino acid sequences of SEQ ID NOs: 12,14,16, and LCDR1, LCDR2, LCDR3 domains, respectively, comprising the amino acid sequences of SEQ ID NOs: 20, 22, 24; and
(b) a second antigen-binding domain that specifically binds human PD-L1 wherein the second antigen-binding domain comprises HCDR1, HCDR2, HCDR3 domains, respectively, comprising the amino acid sequences of SEQ ID NOs: 4,6,8, and LCDR1, LCDR2, LCDR3 domains, respectively, comprising the amino acid sequences of SEQ ID NOs: 20, 22, 24.

20. The isolated bispecific antigen-binding molecule of claim 17, comprising:
(a) a first antigen-binding domain that comprises a HCVR comprising the amino acid sequence of SEQ ID NO: 50, and a LCVR comprising the amino acid sequence of SEQ ID NO: 58; and
(b) a second antigen-binding domain that comprises a HCVR comprising the amino acid sequence of SEQ ID NO: 42, and a LCVR comprising the amino acid sequence of SEQ ID NO: 58.

21. The isolated bispecific antigen-binding molecule of claim 18, comprising:
(a) a first antigen-binding domain that comprises a HCVR comprising the amino acid sequence of SEQ ID NO: 32, and a LCVR comprising the amino acid sequence of SEQ ID NO: 18; and
(b) a second antigen-binding domain that comprises a HCVR comprising the amino acid sequence of SEQ ID NO: 2, and a LCVR comprising the amino acid sequence of SEQ ID NO: 18.

22. The isolated bispecific antigen-binding molecule of claim 19, comprising:
(a) a first antigen-binding domain that comprises a HCVR comprising the amino acid sequence of SEQ ID NO: 10, and a LCVR comprising the amino acid sequence of SEQ ID NO: 18; and
(b) a second antigen-binding domain that comprises a HCVR comprising the amino acid sequence of SEQ ID NO: 2, and a LCVR comprising the amino acid sequence of SEQ ID NO: 18.

23. An isolated bispecific antigen-binding molecule that competes for binding to PD-L1 or binds to the same epitope on PD-L1 as a reference antibody, wherein the reference antibody comprises a first antigen-binding domain comprising an HCVR/LCVR pair comprising the amino acid sequences selected from the group consisting of SEQ ID NOs: 50/58, 32/18 and 10/18 and a second antigen-binding domain comprising an HCVR/LCVR pair comprising the amino acid sequences selected from the group consisting of SEQ ID NOs: 42/58 and 2/18.

24. An isolated bispecific antigen-binding molecule that competes for binding to human CD28 or binds to the same epitope on human CD28 as a reference antibody, wherein the reference antibody comprises a first antigen-binding domain comprising an HCVR/LCVR pair comprising the amino acid sequences selected from the group consisting of SEQ ID NOs: 50/58, 32/18 and 10/18 and a second antigen-binding domain comprising an HCVR/LCVR pair comprising the amino acid sequences selected from the group consisting of SEQ ID NOs: 42/58 and 2/18.

25. The isolated bispecific antigen-binding molecule of claim 1 that is a human bispecific antigen-binding molecule.

26. The isolated bispecific antigen-binding molecule of claim 1 that is a bispecific antibody.

27. The isolated bispecific antigen-binding molecule of 26, wherein the antibody comprises a human IgG heavy chain constant region attached, respectively, to the HCVR of each of the first antigen-binding domain and the second antigen-binding domain.

28. The isolated bispecific antigen-binding molecule of claim 27, wherein the heavy chain constant region is of isotype IgG1.

29. The isolated bispecific antigen-binding molecule of claim 27, wherein the heavy chain constant region is of isotype IgG4.

30. The isolated bispecific antigen-binding molecule of claim 27, wherein the heavy chain constant region attached to the HCVR of the first antigen-binding domain, or the heavy chain constant region attached to the HCVR of the second antigen-binding domain, but not both, contains an amino acid modification that reduces Protein A binding relative to a heavy chain of the same isotype without the modification.

31. The isolated bispecific antigen-binding molecule of claim 30, wherein the modification comprises a H435R substitution (EU numbering) in a heavy chain of isotype IgG1 or IgG4.

32. The isolated bispecific antigen-binding molecule of claim 30, wherein the modification comprises a H435R substitution and a Y436F substitution (EU numbering) in a heavy chain of isotype IgG1 or IgG4.

33. The isolated bispecific antigen-binding molecule of claim 28, wherein the bispecific antibody comprises a chimeric hinge that reduces Fcγ receptor binding relative to a wild-type hinge of the same isotype.

34. The isolated bispecific antigen-binding molecule of claim 27, wherein the antibody comprises a first heavy chain containing the HCVR of the first antigen-binding domain and a second heavy chain containing the HCVR of the second antigen-binding domain, wherein the first heavy chain comprises the amino acid sequence selected from the group consisting of SEQ ID NOs: 68, 40 and 28; and the second heavy chain comprises the amino acid sequence selected from the group consisting of SEQ ID NOs: 66 and 26.

35. The isolated bispecific antigen-binding molecule of claim 34, wherein the antibody comprises a common light chain containing the LCVR of the first and second antigen-binding domains, wherein the common light chain comprises the amino acid sequence selected from the group consisting of SEQ ID NOs: 70 and 30.

36. The isolated bispecific antigen-binding molecule of claim 27, wherein the antibody comprises a first heavy chain containing the HCVR of the first antigen-binding domain and a second heavy chain containing the HCVR of the second antigen-binding domain, wherein the first heavy chain comprises the amino acid sequence of SEQ ID NO: 68 and the second heavy chain comprises the amino acid sequence of SEQ ID NO: 66.

37. The isolated bispecific antigen-binding molecule of claim 36, wherein the antibody comprises a common light chain containing the LCVR of the first and second antigen-binding domains, wherein the common light chain comprises the amino acid sequence of SEQ ID NOs: 70.

38. A bispecific antibody comprising a first antigen-binding domain that binds specifically to human CD28 and a second antigen-binding domain that binds specifically to human PD-L1, wherein the bispecific antibody comprises a first heavy chain comprising the amino acid sequence of SEQ ID NO: 68 paired with a common light chain comprising the amino acid sequence of SEQ ID NO: 70, and a second heavy chain comprising the amino acid sequence of SEQ ID NO: 66 paired with a common light chain comprising the amino acid sequence of SEQ ID NO: 70.

39. A bispecific antibody comprising a first antigen-binding domain that binds specifically to human CD28 and a second antigen-binding domain that binds specifically to human PD-L1, wherein the bispecific antibody comprises a first heavy chain comprising the amino acid sequence of SEQ ID NO: 40 paired with a common light chain comprising the amino acid sequence of SEQ ID NO: 30, and a second heavy chain comprising the amino acid sequence of SEQ ID NO: 26 paired with a common light chain comprising the amino acid sequence of SEQ ID NO: 30.

40. A bispecific antibody comprising a first antigen-binding domain that binds specifically to human CD28 and a second antigen-binding domain that binds specifically to human PD-L1, wherein the bispecific antibody comprises a first heavy chain comprising the amino acid sequence of SEQ ID NO: 28 paired with a common light chain comprising the amino acid sequence of SEQ ID NO: 30, and a second heavy chain comprising the amino acid sequence of SEQ ID NO: 26 paired with a common light chain comprising the amino acid sequence of SEQ ID NO: 30.

41. The bispecific antibody of claim 38 that is a human antibody.

42. A pharmaceutical composition comprising the bispecific antigen-binding molecule of claim 1 and a pharmaceutically acceptable carrier or diluent.

43. A pharmaceutical composition comprising the bispecific antibody of claim 1, and a pharmaceutically acceptable carrier or diluent.

44. A method for making the bispecific antigen-binding molecule of claim 1, comprising:
(a) introducing one or more nucleic acid molecules comprising nucleic acid sequences encoding the immunoglobulin chains of said bispecific antigen-binding molecule into a host cell;
(b) culturing the host cell under conditions favorable to expression of the nucleic acid molecules; and
(c) optionally, isolating the bispecific antigen-binding molecule or immunoglobulin chain from the host cell and/or medium in which the host cell is grown.

45. The method of claim 44, wherein the host cell is a Chinese hamster ovary (CHO) cell.

46. The method of claim 44, further comprising formulating the bispecific antigen-binding molecule as a pharmaceutical composition comprising an acceptable carrier.

47. (canceled)

48. A nucleic acid molecule comprising a nucleotide sequence encoding the bispecific antigen-binding molecule of claim 1, or a set of nucleic acid molecules comprising nucleotide sequences encoding the HCVR of the first antigen-binding domain that specifically binds to human CD28, the HCVR of the second antigen-binding domain that specifically binds to human PD-L1, and the LCVR of claim 1.

49. An expression vector comprising the nucleic acid molecule of claim 48, or a set of expression vectors comprising the set of nucleic acid molecules of claim 48.

50. A host cell comprising the expression vector or set of expression vectors of claim 49.

51. The host cell of claim 50, wherein the host cell is a Chinese hamster ovary (CHO) cell.

52. A method of producing a bispecific antigen-binding molecule that binds to PD-L1 and CD28 comprising:
(a) culturing the host cell of claim 50 under conditions favorable for production of the bispecific antigen-binding molecule; and
(b) optionally, isolating the antigen-binding molecule or immunoglobulin chain from the host cell and/or medium in which the host cell is grown.

53. The method of claim 52, wherein the host cell is a CHO cell.

54. The method of claim 52, further comprising formulating the antigen-binding molecule as a pharmaceutical composition comprising an acceptable carrier.

55-63. (canceled)

64. A method of inhibiting growth of a tumor in a subject, comprising administering an isolated bispecific antigen-binding molecule of claim 1 to the subject.

65. The method of claim 64, wherein the tumor is esophageal carcinoma, lung squamous cell carcinoma, lung adenocarcinoma, cervical squamous cell carcinoma, endometrial adenocarcinoma, bladder urothelial carcinoma, lung cancer, non-small cell lung cancer, colorectal cancer, rectal cancer, endometrial cancer, skin cancer, head & neck squamous cell carcinoma, brain cancer, glioblastoma multiforme, breast cancer, gastroesophageal cancer, gastroesophageal adenocarcinoma, hepatocellular carcinoma, prostate cancer, ovarian cancer, a B cell cancer, a T cell cancer, leukemia, pancreatic cancer, colon cancer, melanoma, basal cell carcinoma, cervical cancer, diffuse large B cell lymphoma, or multiple myeloma.

66. The method of claim 64, wherein the tumor expresses PD-L1.

67. The method of claim 64, further comprising administering a second therapeutic agent or therapeutic regimen.

68. The method of claim 67, wherein the second therapeutic agent or therapeutic regimen comprises a chemotherapeutic drug, a DNA alkylator, an immunomodulator, a proteasome inhibitor, a histone deacetylase inhibitor, radiotherapy, surgery, a stem cell transplant, a bispecific antibody that interacts with a tumor associated antigen (TAA) and a T cell or immune cell antigen, an antibody drug conjugate, an oncolytic virus, a bispecific antibody conjugated to an anti-tumor agent, a VEGF inhibitor, a checkpoint inhibitor, a GITR agonist, a CD27 agonist, a 4-1BB activator, a PD-1 inhibitor, a CTLA-4 inhibitor, an EGFR inhibitor, Ang2 inhibitor, a MUC16 inhibitor, a cancer vaccine, a cytokine, a modified IL2, a modified IL12, IL4 inhibitor, IL6 inhibitor, a corticosteroid, or combinations thereof.

69. The method of claim 68, wherein the T cell or immune cell antigen is CD3.

70. The method of claim 68, wherein the TAA is selected from the group consisting of AFP, ALK, BAGE proteins, BCMA, BIRC5 (survivin), BIRC7, β-catenin, brc-abl, BRCA1, BORIS, CA9, carbonic anhydrase IX, caspase-8, CALR, CCR5, CD19, CD20 (MS4A1), CD22, CD30, CD40, CDK4, CEA, CTLA4, cyclin-B1, CYP1B1, EGFR, EGFRvIII, ErbB2/Her2, ErbB3, ErbB4, ETV6-AML, EpCAM, EphA2, Fra-1, FOLR1, GAGE proteins, GD2, GD3, GloboH, glypican-3, GM3, gp100, Her2, HLA/B-raf, HLA/k-ras, HLA/MAGE-A3, hTERT, LMP2, MAGE proteins, MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSMA (FOLH1), RAGE proteins, Ras, RGS5, Rho, SART-1, SART-3, STEAP1, STEAP2, TAG-72, TGF-β, TMPRSS2, Thompson-nouvelle antigen (Tn), TRP-1, TRP-2, tyrosinase, and uroplakin-3.

71-74. (canceled)


SEQUENCE LISTING (74 sequences)

SEQ ID NO: 1 - DNA, 369 bp, synthetic construct
  misc_feature 1..369: note = Synthetic
  source 1..369: mol_type = other DNA
gaagtgcagctggtggagtctgggggaggcttggttcagcctggcaggtccctaagactctcctgtgcagcctctggattcacctttgatgattatgccttgcactgggtccggcaagctccagggaagggcctggagtgggtctcaggtattagttggactggtggtactatagactatgcggactctgtgaagggccgattcaccatctccagagacaacgccaagaactccctgtatctgcaaatgagcagtctgagaactgaggacacggccatatattactgtacaagagatatccgggggaactggaagtacggaggctggttcgacccctggggccagggaaccctggtcaccgtctcctca

SEQ ID NO: 2 - protein, 123 aa, synthetic construct
  REGION 1..123: note = Synthetic
EVQLVESGGGLVQPGRSLRLSCAASGFTFDDYALHWVRQAPGKGLEWVSGISWTGGTIDYADSVKGRFTISRDNAKNSLYLQMSSLRTEDTAIYYCTRDIRGNWKYGGWFDPWGQGTLVTVSS

SEQ ID NO: 3 - DNA, 24 bp, synthetic construct
  misc_feature 1..24: note = Synthetic
  source 1..24: mol_type = other DNA
ggattcacctttgatgattatgcc

SEQ ID NO: 4 - protein, 8 aa, synthetic construct
  REGION 1..8: note = Synthetic
GFTFDDYA

SEQ ID NO: 5 - DNA, 24 bp, synthetic construct
  misc_feature 1..24: note = Synthetic
  source 1..24: mol_type = other DNA
attagttggactggtggtactata

SEQ ID NO: 6 - protein, 8 aa, synthetic construct
  REGION 1..8: note = Synthetic
ISWTGGTI

SEQ ID NO: 7 - DNA, 48 bp, synthetic construct
  misc_feature 1..48: note = Synthetic
  source 1..48: mol_type = other DNA
acaagagatatccgggggaactggaagtacggaggctggttcgacccc

SEQ ID NO: 8 - protein, 16 aa, synthetic construct
  REGION 1..16: note = Synthetic
TRDIRGNWKYGGWFDP

SEQ ID NO: 9 - DNA, 387 bp, synthetic construct
  misc_feature 1..387: note = Synthetic
  source 1..387: mol_type = other DNA
gaggtgcagctggtggagtctgggggaggcttggtacagccgggggggtccctgagactctcctgtgcagcctctggattcacctttagcacctatgccatgacctgggtccgccaggctccagggaaggggctggagtgggtctcatttattagtggtagtggtgataacacgtattacgcagactccgtgaagggccgattcaccatctccagagacaattccaagaacacgctgtatctgcaaatgaacagcctgagagccgacgattcggccatatatcactgtgcgaaagattccggatattgtagtagtggtgggtgctccctctacttctactacggtatggacgtctggggccaagggaccacggtcaccgtctcctca

SEQ ID NO: 10 - protein, 129 aa, synthetic construct
  REGION 1..129: note = Synthetic
EVQLVESGGGLVQPGGSLRLSCAASGFTFSTYAMTWVRQAPGKGLEWVSFISGSGDNTYYADSVKGRFTISRDNSKNTLYLQMNSLRADDSAIYHCAKDSGYCSSGGCSLYFYYGMDVWGQGTTVTVSS

SEQ ID NO: 11 - DNA, 24 bp, synthetic construct
  misc_feature 1..24: note = Synthetic
  source 1..24: mol_type = other DNA
ggattcacctttagcacctatgcc

SEQ ID NO: 12 - protein, 8 aa, synthetic construct
  REGION 1..8: note = Synthetic
GFTFSTYA

SEQ ID NO: 13 - DNA, 24 bp, synthetic construct
  misc_feature 1..24: note = Synthetic
  source 1..24: mol_type = other DNA
attagtggtagtggtgataacacg

SEQ ID NO: 14 - protein, 8 aa, synthetic construct
  REGION 1..8: note = Synthetic
ISGSGDNT

SEQ ID NO: 15 - DNA, 66 bp, synthetic construct
  misc_feature 1..66: note = Synthetic
  source 1..66: mol_type = other DNA
gcgaaagattccggatattgtagtagtggtgggtgctccctctacttctactacggtatggacgtc

SEQ ID NO: 16 - protein, 22 aa, synthetic construct
  REGION 1..22: note = Synthetic
AKDSGYCSSGGCSLYFYYGMDV

SEQ ID NO: 17 - DNA, 324 bp, synthetic construct
  misc_feature 1..324: note = Synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 18 - protein, 108 aa, synthetic construct
  REGION 1..108: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 19 - DNA, 18 bp, synthetic construct
  misc_feature 1..18: note = Synthetic
  source 1..18: mol_type = other DNA
cagagcattagcagctat

SEQ ID NO: 20 - protein, 6 aa, synthetic construct
  REGION 1..6: note = Synthetic
QSISSY

SEQ ID NO: 21-22 - skipped (2 entries)

SEQ ID NO: 23 - DNA, 30 bp, synthetic construct
  misc_feature 1..30: note = Synthetic
  source 1..30: mol_type = other DNA
caacagagttacagtacccctccgatcacc

SEQ ID NO: 24 - protein, 10 aa, synthetic construct
  REGION 1..10: note = Synthetic
QQSYSTPPIT

SEQ ID NO: 25 - DNA, 1350 bp, synthetic construct
  misc_feature 1..1350: note = Synthetic
  source 1..1350: mol_type = other DNA
gaagtgcagctggtggagtctgggggaggcttggttcagcctggcaggtccctaagactctcctgtgcagcctctggattcacctttgatgattatgccttgcactgggtccggcaagctccagggaagggcctggagtgggtctcaggtattagttggactggtggtactatagactatgcggactctgtgaagggccgattcaccatctccagagacaacgccaagaactccctgtatctgcaaatgagcagtctgagaactgaggacacggccatatattactgtacaagagatatccgggggaactggaagtacggaggctggttcgacccctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 26 - protein, 449 aa, synthetic construct
  REGION 1..449: note = Synthetic
EVQLVESGGGLVQPGRSLRLSCAASGFTFDDYALHWVRQAPGKGLEWVSGISWTGGTIDYADSVKGRFTISRDNAKNSLYLQMSSLRTEDTAIYYCTRDIRGNWKYGGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - DNA, 1368 bp, synthetic construct
  misc_feature 1..1368: note = Synthetic
  source 1..1368: mol_type = other DNA
gaggtgcagctggtggagtctgggggaggcttggtacagccgggggggtccctgagactctcctgtgcagcctctggattcacctttagcacctatgccatgacctgggtccgccaggctccagggaaggggctggagtgggtctcatttattagtggtagtggtgataacacgtattacgcagactccgtgaagggccgattcaccatctccagagacaattccaagaacacgctgtatctgcaaatgaacagcctgagagccgacgattcggccatatatcactgtgcgaaagattccggatattgtagtagtggtgggtgctccctctacttctactacggtatggacgtctggggccaagggaccacggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 28 - protein, 455 aa, synthetic construct
  REGION 1..455: note = Synthetic
EVQLVESGGGLVQPGGSLRLSCAASGFTFSTYAMTWVRQAPGKGLEWVSFISGSGDNTYYADSVKGRFTISRDNSKNTLYLQMNSLRADDSAIYHCAKDSGYCSSGGCSLYFYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 29 - DNA, 648 bp, synthetic construct
  misc_feature 1..648: note = Synthetic
  source 1..648: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 30 - protein, 215 aa, synthetic construct
  REGION 1..215: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 354 bp, synthetic construct
  misc_feature 1..354: note = Synthetic
  source 1..354: mol_type = other DNA
gaggtgcagctggtggagtctgggggaggcttggtccagcctggggggtccctgagactctcctgtgcagcctccggattcaccttcagtaggaataatatgcactgggtccgccaggctccagggaagggactggaatatgtttcaggtattagtagtaatgggggtcgcacatattatgcagactctgtgaagggcagattcaccatctccagagacaattccaagaacacgctgtatcttcaaatgggcggcctgagagctgcggacatggctgtgtatttctgtacgagagatgacgagctgctttcctttgactactggggccagggaaccctggtcactgtctcctca

SEQ ID NO: 32 - protein, 118 aa, synthetic construct
  REGION 1..118: note = Synthetic
EVQLVESGGGLVQPGGSLRLSCAASGFTFSRNNMHWVRQAPGKGLEYVSGISSNGGRTYYADSVKGRFTISRDNSKNTLYLQMGGLRAADMAVYFCTRDDELLSFDYWGQGTLVTVSS

SEQ ID NO: 33 - DNA, 24 bp, synthetic construct
  misc_feature 1..24: note = Synthetic
  source 1..24: mol_type = other DNA
ggattcaccttcagtaggaataat

SEQ ID NO: 34 - protein, 8 aa, synthetic construct
  REGION 1..8: note = Synthetic
GFTFSRNN

SEQ ID NO: 35 - DNA, 24 bp, synthetic construct
  misc_feature 1..24: note = Synthetic
  source 1..24: mol_type = other DNA
attagtagtaatgggggtcgcaca

SEQ ID NO: 36 - protein, 8 aa, synthetic construct
  REGION 1..8: note = Synthetic
ISSNGGRT

SEQ ID NO: 37 - DNA, 33 bp, synthetic construct
  misc_feature 1..33: note = Synthetic
  source 1..33: mol_type = other DNA
acgagagatgacgagctgctttcctttgactac

SEQ ID NO: 38 - protein, 11 aa, synthetic construct
  REGION 1..11: note = Synthetic
TRDDELLSFDY

SEQ ID NO: 39 - DNA, 1335 bp, synthetic construct
  misc_feature 1..1335: note = Synthetic
  source 1..1335: mol_type = other DNA
gaggtgcagctggtggagtctgggggaggcttggtccagcctggggggtccctgagactctcctgtgcagcctccggattcaccttcagtaggaataatatgcactgggtccgccaggctccagggaagggactggaatatgtttcaggtattagtagtaatgggggtcgcacatattatgcagactctgtgaagggcagattcaccatctccagagacaattccaagaacacgctgtatcttcaaatgggcggcctgagagctgcggacatggctgtgtatttctgtacgagagatgacgagctgctttcctttgactactggggccagggaaccctggtcactgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 40 - protein, 444 aa, synthetic construct
  REGION 1..444: note = Synthetic
EVQLVESGGGLVQPGGSLRLSCAASGFTFSRNNMHWVRQAPGKGLEYVSGISSNGGRTYYADSVKGRFTISRDNSKNTLYLQMGGLRAADMAVYFCTRDDELLSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 41 - DNA, 360 bp, synthetic construct
  misc_feature 1..360: note = Synthetic
  source 1..360: mol_type = other DNA
caggtgcagctggtacagtctgggactgaggtgaagaagcctggggcctcagtgaaggtctcctgcaaggcttctggatacaccttcaccgcctactatatgcactgggtgcgacaggcccctggtcaaggacttgactggatgggatggatcagccctaacagtggtttcacaaactatgcacagaagtttcagggcagggtcaccatgaccagggacacgtccatcaacacattttatatggagctgagtggactgagatctgacgacacggccgtatattactgtgcgcgagagggttctactcaccacaattctttcgacccctggggccagggaaccctggtcaccgtctcctca

SEQ ID NO: 42 - protein, 120 aa, synthetic construct
  REGION 1..120: note = Synthetic
QVQLVQSGTEVKKPGASVKVSCKASGYTFTAYYMHWVRQAPGQGLDWMGWISPNSGFTNYAQKFQGRVTMTRDTSINTFYMELSGLRSDDTAVYYCAREGSTHHNSFDPWGQGTLVTVSS

SEQ ID NO: 43 - DNA, 24 bp, synthetic construct
  misc_feature 1..24: note = Synthetic
  source 1..24: mol_type = other DNA
ggatacaccttcaccgcctactat

SEQ ID NO: 44 - protein, 8 aa, synthetic construct
  REGION 1..8: note = Synthetic
GYTFTAYY

SEQ ID NO: 45 - DNA, 24 bp, synthetic construct
  misc_feature 1..24: note = Synthetic
  source 1..24: mol_type = other DNA
atcagccctaacagtggtttcaca

SEQ ID NO: 46 - protein, 8 aa, synthetic construct
  REGION 1..8: note = Synthetic
ISPNSGFT

SEQ ID NO: 47 - DNA, 39 bp, synthetic construct
  misc_feature 1..39: note = Synthetic
  source 1..39: mol_type = other DNA
gcgcgagagggttctactcaccacaattctttcgacccc

SEQ ID NO: 48 - protein, 13 aa, synthetic construct
  REGION 1..13: note = Synthetic
AREGSTHHNSFDP

SEQ ID NO: 49 - DNA, 366 bp, synthetic construct
  misc_feature 1..366: note = Synthetic
  source 1..366: mol_type = other DNA
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggatcacccactacaacccctccctcaagagtcgagtcaccatatcagtagacacgtccaagatccagttctccctgaagctgagttctgtgaccgctgcggacacggccgtgtattactgtgcgagatggggggttcggagggactactactactacggtatggacgtctggggccaagggaccacggtcaccgtctcctca

SEQ ID NO: 50 - protein, 122 aa, synthetic construct
  REGION 1..122: note = Synthetic
QVQLQESGPGLVKPSETLSLTCTVSGGSISSYYWSWIRQPPGKGLEWIGYIYYSGITHYNPSLKSRVTISVDTSKIQFSLKLSSVTAADTAVYYCARWGVRRDYYYYGMDVWGQGTTVTVSS

SEQ ID NO: 51 - DNA, 24 bp, synthetic construct
  misc_feature 1..24: note = Synthetic
  source 1..24: mol_type = other DNA
ggtggctccatcagtagttactac

SEQ ID NO: 52 - protein, 8 aa, synthetic construct
  REGION 1..8: note = Synthetic
GGSISSYY

SEQ ID NO: 53 - DNA, 21 bp, synthetic construct
  misc_feature 1..21: note = Synthetic
  source 1..21: mol_type = other DNA
atctattacagtgggatcacc

SEQ ID NO: 54 - protein, 7 aa, synthetic construct
  REGION 1..7: note = Synthetic
IYYSGIT

SEQ ID NO: 55 - DNA, 48 bp, synthetic construct
  misc_feature 1..48: note = Synthetic
  source 1..48: mol_type = other DNA
gcgagatggggggttcggagggactactactactacggtatggacgtc

SEQ ID NO: 56 - protein, 16 aa, synthetic construct
  REGION 1..16: note = Synthetic
ARWGVRRDYYYYGMDV

SEQ ID NO: 57 - DNA, 324 bp, synthetic construct
  misc_feature 1..324: note = Synthetic
  source 1..324: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 58 - protein, 108 aa, synthetic construct
  REGION 1..108: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 59 - DNA, 21 bp, synthetic construct
  misc_feature 1..21: note = Synthetic
  source 1..21: mol_type = other DNA
cagtcagtctctagctcttat

SEQ ID NO: 60 - protein, 7 aa, synthetic construct
  REGION 1..7: note = Synthetic
QSVSSSY

SEQ ID NO: 61-62 - skipped (2 entries)

SEQ ID NO: 63 - DNA, 27 bp, synthetic construct
  misc_feature 1..27: note = Synthetic
  source 1..27: mol_type = other DNA
caacagtacggaagcagcccgtggacg

SEQ ID NO: 64 - protein, 9 aa, synthetic construct
  REGION 1..9: note = Synthetic
QQYGSSPWT

SEQ ID NO: 65 - DNA, 1341 bp, synthetic construct
  misc_feature 1..1341: note = Synthetic
  source 1..1341: mol_type = other DNA
caggtgcagctggtacagtctgggactgaggtgaagaagcctggggcctcagtgaaggtctcctgcaaggcttctggatacaccttcaccgcctactatatgcactgggtgcgacaggcccctggtcaaggacttgactggatgggatggatcagccctaacagtggtttcacaaactatgcacagaagtttcagggcagggtcaccatgaccagggacacgtccatcaacacattttatatggagctgagtggactgagatctgacgacacggccgtatattactgtgcgcgagagggttctactcaccacaattctttcgacccctggggccagggaaccctggtcaccgtctcctcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 66 - protein, 446 aa, synthetic construct
  REGION 1..446: note = Synthetic
QVQLVQSGTEVKKPGASVKVSCKASGYTFTAYYMHWVRQAPGQGLDWMGWISPNSGFTNYAQKFQGRVTMTRDTSINTFYMELSGLRSDDTAVYYCAREGSTHHNSFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 67 - DNA, 1347 bp, synthetic construct
  misc_feature 1..1347: note = Synthetic
  source 1..1347: mol_type = other DNA
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggatcacccactacaacccctccctcaagagtcgagtcaccatatcagtagacacgtccaagatccagttctccctgaagctgagttctgtgaccgctgcggacacggccgtgtattactgtgcgagatggggggttcggagggactactactactacggtatggacgtctggggccaagggaccacggtcaccgtctcctcagcctctacaaagggaccttctgtgtttcctctggctccttgttctagatctacatctgaatctacagctgctctgggatgtctggtgaaggattattttcctgaacctgtgacagtgtcttggaattctggagctctgacatctggagtgcatacatttcctgctgtgctgcagtcttctggactgtattctctgtcttctgtggtgacagtgccttcttcttctctgggaacaaagacatatacatgtaatgtggatcataagccttctaatacaaaggtggataagagagtggaatctaagtatggacctccttgtcctccttgtcctgctcctcctgtggctggaccttctgtgtttctgtttcctcctaagcctaaggatacactgatgatctctagaacacctgaagtgacatgtgtggtggtggatgtgtctcaggaagatcctgaagtgcagtttaattggtatgtggatggagtggaagtgcataatgctaagacaaagcctagagaagaacagtttaattctacatatagagtggtgtctgtgctgacagtgctgcatcaggattggctgaatggaaaggaatataagtgtaaggtgtctaataagggactgccttcttctatcgaaaagacaatctctaaggctaagggacagcctagagaacctcaggtgtatacactgcctccttctcaggaagaaatgacaaagaatcaggtgtctctgacatgtctggtgaagggattttatccttctgatatcgctgtggaatgggaatctaatggacagcctgaaaataattataagacaacacctcctgtgctggattctgatggatctttttttctgtattctagactgacagtggataagtctagatggcaggaaggaaatgtgttttcttgttctgtgatgcatgaagctctgcataatagatttacacagaagtctctgtctctgtctcctggaaagtag

SEQ ID NO: 68 - protein, 448 aa, synthetic construct
  REGION 1..448: note = Synthetic
QVQLQESGPGLVKPSETLSLTCTVSGGSISSYYWSWIRQPPGKGLEWIGYIYYSGITHYNPSLKSRVTISVDTSKIQFSLKLSSVTAADTAVYYCARWGVRRDYYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 69 - DNA, 648 bp, synthetic construct
  misc_feature 1..648: note = Synthetic
  source 1..648: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 70 - protein, 215 aa, synthetic construct
  REGION 1..215: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 249 aa, synthetic construct
  REGION 1..249: note = Synthetic
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTEQKLISEEDLGGEQKLISEEDLHHHHHH

SEQ ID NO: 72 - protein, 367 aa, synthetic construct
  REGION 1..367: note = Synthetic
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 73 - protein, 448 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 454 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK